(19) United States
(12) Patent Application Publication
(10) Pub. No.: US 2023/0265068 A1
(43) Pub. Date: Aug. 24, 2023

(54) TRANSIENT RECEPTOR POTENTIAL CANONICAL 3 INHIBITORS AND METHODS OF USE THEREOF

(71) Applicant: University of Tennessee Research Foundation, Memphis, TN (US)

(72) Inventors: Wei Li, Germantown, TN (US); Julio Cordero-Morales, Germantown, TN (US); Jianxiong Jiang, Memphis, TN (US); Zhongzhi Wu, Collierville, TN (US); Francesca Liao, Memphis, TN (US); Catherine Kaczorowski, Memphis, TN (US)

(73) Assignee: University of Tennessee Research Foundation, Memphis, TN (US)

(21) Appl. No.: 18/299,562

(22) Filed: Apr. 12, 2023

Related U.S. Application Data
(63) Continuation of application No. PCT/US21/54579, filed on Oct. 12, 2021.
(60) Provisional application No. 63/090,486, filed on Oct. 12, 2020.

(51) Int. Cl. C07D 401/10; A61P 25/08

(52) U.S. Cl. CPC C07D 401/10; A61P 25/08

(57) ABSTRACT

Provided herein are compounds, pharmaceutical compositions comprising such compounds, and methods of using such compounds to treat diseases or disorders associated with TRPC3 activity.

### CROSS REFERENCE TO RELATED APPLICATIONS

This application is a national stage application, filed under 35 U.S.C. § 371, of International Patent Application No. PCT/US2021/054579, filed on Oct. 12, 2021, which claims the benefit of U.S. Provisional Application No. 63/090,486, filed Oct. 12, 2020, which is hereby incorporated herein by reference in its entirety.

### STATEMENT OF FEDERALLY SPONSORED RESEARCH

This invention was made with government support under GM125629 and AG049772 awarded by National Institutes of Health. The government has certain rights in the invention.

### FIELD OF THE INVENTION

This invention relates to novel chemical compositions used for therapeutic purposes. More specifically, it related to novel transient receptor potential canonical channel inhibitors and methods of use thereof.

### BACKGROUND OF THE INVENTION

Transient receptor potential canonical (TRPC) channels are ubiquitously expressed in vertebrate cells.¹ TRPC channels consist of seven members designated TRPC1-7, although only six TRPC channels are expressed in humans because TRPC2 is a pseudogene. The other six channels are further divided into two groups, TRPC1/4/5 and TRPC3/6/7, based on amino acid sequence homology and functional similarities. As nonselective cationic channels, they control the influx of Ca²⁺ and other cations like Na⁺ in response to activation of phospholipase C-coupled plasma membrane receptors and, thus, play critical roles in the regulation of intracellular Ca²⁺ concentration by hormones and growth factors.²⁻³ Among the TRPC family, TRPC3 plays a prominent functional role in basic cellular responses including proliferation, differentiation and death in response to various environmental stimuli,⁴⁻⁶ implying a variety of diverse biological functions.² TRPC3 is the most abundant TRPC channel in brain and TRPC channels are expressed to the greatest extent in the central nervous system.⁷ Recent studies have suggested that TRPC3 channels are critical for the signaling cascade of brain derived neurotrophic factor,⁸⁻⁹ which has been postulated as a critical contributor to Alzheimer's disease.¹⁰ TRPC3 is also expressed in cardiovascular cells and TRPC3 overexpression has been found to be involved in adverse mechanical stress responses, hypertrophy, and heart failure.¹¹ In the immune system, TRPC3 was suggested to contribute to the restoration of Ca²⁺ influx and activation of T-cells, and thus facilitate the response to antigen stimulation.¹²⁻¹³ Additionally, TRPC3 is involved in the proliferation and migration of a variety of tumor cells, including melanoma, lung and breast cancers.¹⁴⁻¹⁶

The current understanding of the role of TRPC3 in most pathological studies suggests that channel blockers may be suitable for the treatment of diseases like coronary stenosis and melanoma.^{14,17} Compared to nonselective channel blockers such as La³⁺, Gd³⁺, verapamil or SKF96365,¹⁸⁻¹⁹ selective TRPC3 inhibitors show superiority in selectively dissecting the function and multiple roles of the native TRPC3, and are therefore suitable for the development of therapeutic applications. Over the past decades, several small molecular TRPC3 inhibitors have been reported. Although the impact of Pyr3 on the suppression of TRPC3 is relatively mild, with a reported IC₅₀ of 0.7 μM,²⁰ it shows high specificity for TRPC3 whereas most other inhibitors possess comparable or higher potency against TRPC6. As a powerful tool to study in vivo function of TRPC3, several pharmacological studies suggest the potential of Pyr3 in treatment of TRPC3-related diseases such as cardiac hypertrophy¹⁹ and smooth muscle proliferation¹⁷.

Preliminary structure-activity relationship (SAR) studies showed that the trichloroacrylic amide group in Pyr3 is critical for the observed subtype specificity.²⁰ However, as a highly reactive alkylating moiety, this group may have significant potential toxicity. Furthermore, Pyr3 has poor stability in vivo because the ester group is easily hydrolyzed, leading to an acid derivative Pyr8, which shows little TRPC3 inhibition.²¹ Therefore, there is a need for more stable and selective TRPC3 inhibitors with low toxicity for their potential therapies in diseases such as cardiac hypertrophy,¹⁹ cancer and Alzheimer's disease.¹⁰

### SUMMARY

Recent studies have suggested that TRPC3 channels are critical for the signaling cascade of brain derived neurotrophic factor,⁸⁻⁹ which has been postulated as a critical contributor to Alzheimer's disease.¹⁰ The TRPC3 inhibitors disclosed herein are novel therapeutics for treatment of Alzheimer's disease. This disclosure relates to such compounds, pharmaceutical compositions comprising such compounds, and uses related thereto. In an aspect, provided herein are compounds of Formula (III), (IV), and/or (V):

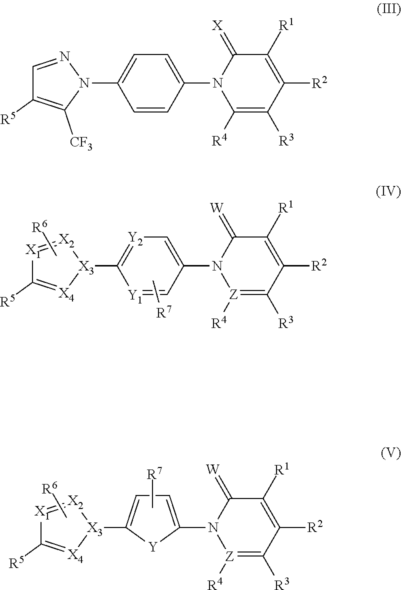

including salts and prodrugs thereof, wherein the substituents are reported herein.

In certain embodiments, this disclosure relates to methods of treating cardiac hypertrophy, cancer, neurological diseases/disorders such as epilepsy, and/or neurodegenerative diseases, such as Alzheimer's Disease, comprising administering a therapeutically effective amount of a compound disclosed herein to a subject in need thereof.

### BRIEF DESCRIPTION OF FIGURES

FIGS. 1A-1C show inhibition of TRPC3 mediated currents by synthesized compounds. (FIG. 1A) Percentage of peak current inhibited by the synthesized compounds acting in the presence of the agonist GSK1702934A of HEK293 cells transfected with hTRPC3. Bars are mean±SD. n is denoted above the x-axis. FIGS. 1B and 1C show normalized current densities inhibited by different concentrations of compound 22 (FIG. 1B) and Pyr3 (FIG. 1C) acting in the presence of the agonist GSK1702934A of HEK293 cells transfected with hTRPC3. Circles are mean±SD. FIGS. 1D-1I show inhibition of TRPC3-mediated currents by compound 22 and Pyr3 (serving as the reference control). Representative dose-response traces of hTRPC3 currents activated by 1 μM GSK₁₇₀, and the calculated IC₅₀ values of compound 22 without extracellular Ca²⁺ (FIG. 1E), compound 22 in the presence of 2 mM extracellular Ca²⁺ (FIG. 1E, FIG. 1F), Pyr3 without extracellular Ca²⁺ (FIG. 1H), and Pyr3 in the presence of 2 mM extracellular Ca²⁺ (FIG. 1H, FIG. 1F), respectively. Circles represent mean±SD. GSK₁₇₀: GSK1702934A.

FIGS. 2A-2H show studies of compound 22 as a selective inhibitor of the TRPC subfamily. (FIGS. A-G) Representative whole-cell recordings of HEK293 cells over-expressing hTRPA1 (FIG. 2A), rTRPV1 (FIG. 2B), rTRPV4 (FIG. 2C), hTRPC3 (FIG. 2D), mmTRPC6 (FIG. 2E), hTRPC7 (FIG. 2F), and hTRPM8 (FIG. 2G). Currents were evoked with high concentrations of the agonist and challenged with compound 22 (10 μM) in the presence of the respective agonist. (FIG. 2H) Percentage of peak current blocked by compound 22 (10 PM) in the presence of each agonist. Bars are mean±SD. n is denoted above the x-axis.

FIGS. 3A-3B show preliminary LC/MS analyses of the prepared control (FIG. 3A) and the purified compound 22-TRPC3 complexes (FIG. 3B) together with support the direct binding of compound 22 to TRPC3 proteins. Spectra of MS data collected from 2.5 minutes to avoid buffer salts to MS.

FIGS. 4A-4B show the maximum tolerated dose study in mice. (FIG. 4A) Mouse survival curves. (FIG. 4B) Mean percent change in mouse body weight±SEM relative to body weight at the time of initiating drug treatment. All mice received one dose of compound 79 or compound 22 (n=5) daily for 5 consecutive days. Dashed lines indicate weight relative to the baseline (y axis=0) and 15% weight loss. FIG. 4C shows the safety profile of compound 22 compared to compound 79.

FIG. 5 shows the effect on dendritic spine morphology. To visualize dendritic spines, 14 DIV hippocampal neurons at low density (5,000 cells per 35 mm dish) were exposed to 7PA2 conditioned media (CM), which contains naturally secreted Aβ, or CHO control media or for 16 h, with or without cotreatment with the two inhibitor compounds, followed by immunocytochemistry with MAP2 and DAPI counterstaining.

FIG. 6 shows brain concentrations of compound 22 after systemic dose in mice (20 mg/kg, i.v.). The brain-to-plasma ratio was ˜0.15 at 1 h after injection. Data are shown as mean±SD (n=3). Its TRPC3 IC₅₀ (0.51 μM or 227 ng/ml) is displayed to show duration of drug exposure in the brain following each dose.

FIGS. 7A-7F show that TRPC3 inhibition reduces vulnerability of mice to pilocarpine-induced seizures. (FIG. 7A) Mice were treated by pilocarpine (220 mg/kg, i.p.) for seizure induction, and behavioral seizures were scored every 5 min based a modified Racine scale. After stage-2 behavioral seizures were established (usually about 15-20 min after pilocarpine injection), mice were treated by either vehicle or compound 22 (100 mg/kg, i.p.). Compound 22 reduced the scores of pilocarpine-induced seizures (p<0.001, two-way ANOVA with post-hoc Bonferroni's multiple comparisons test). (FIG. 7B) Maximal seizure scores in mice after pilocarpine injection (p<0.0001, t-test). (FIG. 7C) Latency to partial (stage-3) seizures (p=0.0002, t-test). (FIG. 7D) Latency to generalized (stage-4) seizures (p=0.0001, t-test). (FIG. 7E) Percentage of animals that experienced stage-5 seizures or SE (p<0.0001, Fisher's exact test). (FIG. 7F) Percentage of animals survived pilocarpine-induced seizures (p=0.1649, Fisher's exact test). Data are presented as mean+/±SEM.

FIGS. 8A-8B show the inhibition of TRPC3 channels by compound 22 reduces susceptibility of animals to PTZ-induced seizures. Young adult C57BL/6 mice (male, 8-9 weeks) were first treated by compound 22 (100 mg/kg, i.p.), and 30 minutes later by pentylenetetrazole (PTZ, 80±5 mg/kg, s.c.) for the induction seizures. (FIG. 8A) Percentages of mice reaching myoclonic jerk (MJ, Left) and generalized tonic-clonic seizure (GTCS, Right) were compared between vehicle and compound 22-treated mice by Mantel-Cox log-rank test. (FIG. 8B) Latencies to the first MJ (Left) and first GTCS (Right) were compared between vehicle and compound 22 by t-test. Data are shown as mean+SEM (number of mice N=9 for vehicle; 10 for compound 22).

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, particular methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

### I. Definitions

It must be noted that, as used in the specification and the appended claims, the singular forms “a,” “an,” and “the” include plural referents unless the context clearly dictates otherwise.

Regarding chemical structure, it is understood that claiming compounds that are racemic encompasses all of the isomers, tautomers, enantiomers, or diastereomers unless otherwise specified to be a composition of excess of a specific isomer. For instance, an isomer/enantiomer can, in some embodiments, be provided substantially free of the corresponding enantiomer, and can also be referred to as “optically enriched,” “enantiomerically enriched,” “enantiomerically pure” and “non-racemic,” as used interchangeably herein. These terms refer to compositions in which the amount of one enantiomer is greater than the amount of that one enantiomer in a control mixture of the racemic composition (e.g., greater than 1:1 by weight). For example, an enantiomerically enriched preparation of the S enantiomer, means a preparation of the compound having greater than about 50% by weight of the S enantiomer relative to the total weight of the preparation (e.g., total weight of S and R isomers) such as at least about 75% by weight, further such as at least about 80% by weight. In some embodiments, the enrichment can be much greater than about 80% by weight, providing a “substantially enantiomerically enriched,” “substantially enantiomerically pure” or a “substantially non-racemic” preparation, which refers to preparations of compositions which have at least about 85% by weight of one enantiomer relative to the total weight of the preparation, such as at least about 90% by weight, and further such as at least about 95% by weight. In certain embodiments, the compound provided herein is made up of at least about 90% by weight of one enantiomer. In other embodiments, the compound is made up of at least about 95%, about 98%, or about 99% by weight of one enantiomer.

In certain embodiments, the pharmaceutically acceptable form is a tautomer. As used herein, the term “tautomer” is a type of isomer that includes two or more interconvertable compounds resulting from at least one formal migration of a hydrogen atom and at least one change in valency (e.g., a single bond to a double bond, a triple bond to a double bond, or a triple bond to a single bond, or vice versa). “Tautomerization” includes prototropic or proton-shift tautomerization, which is considered a subset of acid-base chemistry. “Prototropic tautomerization” or “proton-shift tautomerization” involves the migration of a proton accompanied by changes in bond order. The exact ratio of the tautomers depends on several factors, including temperature, solvent, and Ph. Where tautomerization is possible (e.g., in solution), a chemical equilibrium of tautomers can be reached. Tautomerizations (i.e., the reaction providing a tautomeric pair) can be catalyzed by acid or base, or can occur without the action or presence of an external agent. Exemplary tautomerizations include, but are not limited to, keto-enol; amide-imide; lactam-lactim; enamine-imine; and enamine-(a different) enamine tautomerizations. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

The disclosure also embraces isotopically labeled compounds which are identical to those recited herein, except that one or more atoms are replaced by an atom having an atomic mass or mass number different from the atomic mass or mass number usually found in nature. Examples of isotopes that can be incorporated into disclosed compounds include isotopes of hydrogen, carbon, nitrogen, oxygen, phosphorus, sulfur, fluorine, and chlorine, such as, e.g., ²H, ³, ¹³C, ¹⁴C, ¹⁵N, ¹⁸O, ¹⁷O, ³¹P, ³²P, ³⁵S, ¹⁸F, and ³⁶Cl, respectively. Certain isotopically-labeled disclosed compounds (e.g., those labeled with ³H and/or ¹⁴C) are useful in compound and/or substrate tissue distribution assays. Tritiated (i.e., ³H) and carbon-14 (i.e., ¹⁴C) isotopes can allow for ease of preparation and detectability. Further, substitution with heavier isotopes such as deuterium (i.e., ²H) can afford certain therapeutic advantages resulting from greater metabolic stability (e.g., increased in vivo half-life or reduced dosage requirements). Isotopically labeled disclosed compounds can generally be prepared by substituting an isotopically labeled reagent for a non-isotopically labeled reagent. In some embodiments, provided herein are compounds that can also contain unnatural proportions of atomic isotopes at one or more of atoms that constitute such compounds. All isotopic variations of the compounds as disclosed herein, whether radioactive or not, are encompassed within the scope of the present disclosure.

The term “prodrug” refers any covalently bonded carriers, which release the active compound in vivo when such prodrug is administered to a subject. Prodrugs of an active compound, as described herein, can be prepared by modifying functional groups present in the active compound in such a way that the modifications are cleaved, either in routine manipulation or in vivo, to the parent active compound. Prodrugs include compounds wherein a carboxyl, hydroxy, amino or mercapto group is bonded to any group that, when the prodrug of the active compound is administered to a subject, cleaves to form a free hydroxy, free carboxyl, free amino or free mercapto group, respectively. Examples of prodrugs include, but are not limited to, carboxyl esters, acetate, formate and benzoate derivatives of an alcohol or acetamide, formamide and benzamide derivatives of an amine functional group in the active compound and the like.

“Ester” refers to esters of acidic groups, including, but not limited to, carboxylic acids, phosphoric acids, phosphinic acids, sulfonic acids, sulfinic acids, and boronic acids, e.g., a radical of formula —COOR, where R is selected from alkyl, alkenyl, alkynyl, haloalkyl, heteroalkyl (bonded through a chain carbon), cycloalkyl, cycloalkylalkyl, aryl, aralkyl, heterocycloalkyl (bonded through a ring carbon), heterocycloalkylalkyl, heteroaryl (bonded through a ring carbon), and heteroarylalkyl. Any amine, hydroxy, or carboxyl side chain on the compounds described herein can be esterified. The procedures and specific groups to make such esters are known to those of skill in the art and can readily be found in reference sources such as Greene and Wuts, Protective Groups in Organic Synthesis, 4^{th} Ed., John Wiley & Sons, New York, N.Y., 2006, which is incorporated herein by reference in its entirety. Unless stated otherwise in the specification, an ester group can be optionally substituted by one or more substituents

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRₐR_{b}, —NRₐC(═O)R_{b}, —NRₐC(═O)NRₐNR_{b}, —NRₐC(═O)OR_{b}, —NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ and —S(═O)₂ORₐ. Rₐ and R_{b} in this context may be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, and heteroarylalkyl.

As used herein, “alkyl” means a noncyclic straight chain or branched, unsaturated or saturated hydrocarbon such as those containing from 1 to 10 carbon atoms (C₁-C₁₀)alkyl. In certain embodiments, any alkyl is a (C₁-C₆)alkyl, or any group containing an alkyl reported herein, e.g., a (C₁-C₆)alkoxy. Representative saturated straight chain alkyls include methyl, ethyl, n-propyl, n-butyl, n-pentyl, n-hexyl, n-septyl, n-octyl, n-nonyl, and the like; while saturated branched alkyls include isopropyl, sec-butyl, isobutyl, tert-butyl, isopentyl, and the like. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

Non-aromatic mono or polycyclic alkyls are referred to herein as “carbocycles” or “carbocyclyl” groups. Representative saturated carbocycles include cyclopropyl, cyclobutyl, cyclopentyl, cyclohexyl, and the like; while unsaturated carbocycles include cyclopentenyl and cyclohexenyl, and the like.

“Heterocarbocycles” or heterocarbocyclyl” groups are carbocycles which contain from 1 to 4 heteroatoms independently selected from nitrogen, oxygen and sulfur which may be saturated or unsaturated (but not aromatic), monocyclic or polycyclic, and wherein the nitrogen and sulfur heteroatoms may be optionally oxidized, and the nitrogen heteroatom may be optionally quaternized. Heterocarbocycles include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

The term “aryl” refers to aromatic homocyclic (i.e., hydrocarbon) mono-, bi- or tricyclic ring-containing groups preferably having 6 to 12 members such as phenyl, naphthyl and biphenyl. In an embodiment, aryl is phenyl. The term “substituted aryl” refers to aryl groups substituted with one or more groups, preferably selected from alkyl, substituted alkyl, alkenyl (optionally substituted), aryl (optionally substituted), heterocyclo (optionally substituted), halo, hydroxy, alkoxy (optionally substituted), aryloxy (optionally substituted), alkanoyl (optionally substituted), aroyl, (optionally substituted), alkylester (optionally substituted), arylester (optionally substituted), cyano, nitro, amino, substituted amino, amido, lactam, urea, urethane, sulfonyl, and, the like, where optionally one or more pair of substituents together with the atoms to which they are bonded form a 3 to 7 member ring.

As used herein, “heteroaryl” or “heteroaromatic” refers an aromatic heterocarbocycle having 1 to 4 heteroatoms selected from nitrogen, oxygen and sulfur, and containing at least 1 carbon atom, including both mono- and polycyclic ring systems. Polycyclic ring systems may, but are not required to, contain one or more non-aromatic rings, as long as one of the rings is aromatic. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl. It is contemplated that the use of the term “heteroaryl” includes N-alkylated derivatives such as a 1-methylimidazol-5-yl substituent.

As used herein, “heterocycle” or “heterocyclyl” refers to mono- and polycyclic ring systems having 1 to 4 heteroatoms selected from nitrogen, oxygen and sulfur, and containing at least 1 carbon atom. The mono- and polycyclic ring systems may be aromatic, non-aromatic or mixtures of aromatic and non-aromatic rings. Heterocycle includes heterocarbocycles, heteroaryls, and the like.

“Alkylthio” refers to an alkyl group as defined above with the indicated number of carbon atoms attached through a sulfur bridge. An example of an alkylthio is methylthio, (i.e., —S—CH3).

“Alkoxy” refers to an alkyl group as defined above with the indicated number of carbon atoms attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. In an embodiment, alkoxy groups are methoxy, ethoxy, n-propoxy, i-propoxy, n-butoxy, s-butoxy, t-butoxy.

“Alkylamino” refers an alkyl group as defined above with the indicated number of carbon atoms attached through an amino bridge. An example of an alkylamino is methylamino, (i.e., —NH—CH₃).

“Alkanoyl” refers to an alkyl as defined above with the indicated number of carbon atoms attached through a carbonyl bride (i.e., —(C═O)alkyl).

“Alkylsulfonyl” refers to an alkyl as defined above with the indicated number of carbon atoms attached through a sulfonyl bridge (i.e., —S(═O)₂alkyl) such as mesyl and the like, and “Arylsulfonyl” refers to an aryl attached through a sulfonyl bridge (i.e., —S(═O)₂aryl).

“Alkylsulfamoyl” refers to an alkyl as defined above with the indicated number of carbon atoms attached through a sulfamoyl bridge (i.e., —NHS(═O)₂alkyl), and an “Arylsulfamoyl” refers to an alkyl attached through a sulfamoyl bridge (i.e., —NHS(═O)₂aryl).

“Alkylsulfinyl” refers to an alkyl as defined above with the indicated number of carbon atoms attached through a sulfinyl bridge (i.e., —S(═O)alkyl).

The terms “cycloalkyl” and “cycloalkenyl” refer to mono-, bi-, or tri homocyclic ring groups of 3 to 15 carbon atoms which are, respectively, fully saturated, and partially unsaturated. The term “cycloalkenyl” includes bi- and tricyclic ring systems that are not aromatic as a whole, but contain aromatic portions (e.g., fluorene, tetrahydronapthalene, dihydroindene, and the like). The rings of multi-ring cycloalkyl groups may be either fused, bridged and/or joined through one or more spiro unions. The terms “substituted cycloalkyl” and “substituted cycloalkenyl” refer, respectively, to cycloalkyl and cycloalkenyl groups substituted with one or more groups, preferably selected from aryl, substituted aryl, heterocyclo, substituted heterocyclo, carbocyclo, substituted carbocyclo, halo, hydroxy, alkoxy (optionally substituted), aryloxy (optionally substituted), alkylester (optionally substituted), arylester (optionally substituted), alkanoyl (optionally substituted), aryol (optionally substituted), cyano, nitro, amino, substituted amino, amido, lactam, urea, urethane, sulfonyl, and the like.

The terms “halogen” and “halo” refer to fluorine, chlorine, bromine, and iodine.

The term “carbamoyl” refers to a functional group having the formula —OC(O)NH₂ or, alternatively, —NHC(O)OH.

The term “boronic acid” refers to a functional group having the formula —B(OH)₂.

The term “boronic ester” refers to a functional group having the formula —B(Oalkyl)₂ wherein alkyl is defined above and the two alkyl groups may be connected to form a cyclic boronic ester.

The term “carboxy” refers to the functional group —C(O)—.

The term “hydroxy” refers to an alcohol functional group having the formula —OH.

The term “nitro” refers to a functional group having the formula —NO₂, wherein the nitrogen atom is positively charged and singly bound to a negatively charged oxygen atom and doubly bound to a second oxygen atom.

The term “mercapto” is synonymous with the term “thio,” which refers to a functional group having the formula —SH.

The term “cyano” refers to a functional group having the formula —CN, wherein carbon is triply bound to nitrogen.

The term “sulfamoyl” refers to a functional group having the formula —SO₂NH₂, wherein the sulfur atom is doubly bound to two oxygen atoms and singly bound to nitrogen.

An unspecified “R” group is a hydrogen, lower alkyl, or aryl all of which may be optionally substituted with one or more substituents. Throughout the specification, groups and substituents thereof may be chosen to provide stable moieties and compounds.

As used herein, “subject” refers to any animal, preferably a human patient, livestock, or domestic pet.

As used herein, the terms “treat” and “treating” are not limited to the case where the subject (e.g., patient) is cured and the disease is eradicated. Rather, embodiments of the present disclosure also contemplate treatment that merely reduces symptoms, and/or delays disease progression. The term “effective amount” or “therapeutically effective amount” refers to that amount of a compound or pharmaceutical composition described herein that is sufficient to affect the intended application including, but not limited to, disease treatment, as illustrated below. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells. The specific dose will vary depending on, for example, the particular compounds chosen, the dosing regimen to be followed, whether it is administered in combination with other agents, timing of administration, the tissue to which it is administered, and the physical delivery system in which it is carried.

As used herein, the term “combination with” when used to describe administration with an additional treatment means that the agent may be administered prior to, together with, or after the additional treatment, or a combination thereof.

### II. Methods of Treatment

In some embodiments, provided herein is a method of treating neurological diseases and/or neurodegenerative diseases. In an another aspect, provided herein is a method of treating Alzheimer's disease, which includes treating or ameliorating one or more symptoms associated with Alzheimer's disease in a subject in need thereof, including administering to the subject a therapeutically effective amount of a compound or composition disclosed herein.

In an embodiment, the subject is at risk of, exhibiting symptoms of, or diagnosed with memory loss, early Alzheimer. In certain embodiments, the compound is administered in combination with another neural protecting agent.

In another aspect, provided herein is a method of treating epilepsy disease in a subject in need thereof, including administering to the subject a therapeutically effective amount of a compound or composition disclosed herein. Epilepsy is one of the most prevalent neurological disorders, affecting more than 1% of the population worldwide. Despite advances in seizure management over the past few decades, there are still 30-40% of epilepsy patients who do not adequately respond to current anti-seizure drugs (ASDs). In addition, over 80% of epilepsy patients experience adverse effects to existing ASDs, in half of whom the quality of life is substantially impaired. None of the existing FDA-approved drugs haves been shown to prevent the development of epilepsy in people at high risks or to modify seizure progression in those diagnosed with epilepsy. Therefore, developing a new targeted therapy for epilepsy that can be more efficacious is highly significant to this unmet need. Epilepsies are characterized by chronic and paroxysmal alterations in neurologic function associated with deviate changes in the electrical activity of the brain. Epileptic seizures are generally classified as Primary Generalized Seizures (convulsive or non-convulsive), which include tonic-clonic (grand-mal), tonic, absence (petit mal), atypical absence, myoclonic, atonic, infantile spasms and partial or foal seizures; or Simple partial seizures (without impaired consciousness), with motor symptoms, with somatosensory or special sensory symptoms, without autonomic symptoms, with psychological symptoms and complex partial seizures (with impaired consciousness).

In another aspect, provided herein is a method of in inhibiting a protein in a subject in need thereof, comprising administering to the subject a therapeutically effective amount of a compound or composition disclosed herein.

In an embodiment, the protein is TRPC3.

The effective amount of the present compounds depends on many factors, including the indication being treated, the route of administration, co-administration of other therapeutic compositions, and the overall condition of the patient.

In general, treatment regimens utilizing compounds include administration of from about 0.1 mg to about 300 mg of the compounds per kilogram body weight of the recipient per day in multiple doses or in a single dose. In some embodiments, a suitable dose may be in the range of 0.1 to 300 mg per kilogram body weight of the recipient per day, optionally in the range of 6 to 150 mg per kilogram body weight per day, optionally in the range of 15 to 100 mg per kilogram body weight per day, optionally in the range of 15 to 80 mg per kilogram body weight per day, optionally in the range of 15 to 50 mg per kilogram body weight per day, and optionally in the range of 15 to 30 mg per kilogram body weight per day. The desired dose may be presented as two, three, four, five or six or more sub-doses administered at appropriate intervals throughout the day. These sub-doses may be administered in unit dosage forms, for example, containing 10 to 2000 mg, optionally 10 to 1500 mg, optionally 20 to 1000 mg, and optionally 50 to 700 mg of the compounds per unit dosage form.

In certain embodiments, a compound or composition as disclosed herein is used in the production of a medicament for use in treating Alzheimer's disease or treating or ameliorating one or more symptoms associated with Alzheimer's disease. For example, the compounds and/or their pharmaceutically acceptable salts can be administered in the form of a pharmaceutical composition in association with one or more pharmaceutically acceptable excipients, such as the pharmaceutical compositions described below. The choice of the pharmaceutically acceptable excipients will to a large extent depend on factors such as the particular mode of administration, the effect of the excipient on solubility and stability, and the nature of the dosage form.

### A. Compounds

In certain embodiments, this disclosure relates to therapeutically beneficial pyrazole derivatives as compounds of this disclosure. In certain embodiments, the pyrazole compounds are compounds disclosed herein optionally substituted with one or more substituents, or derivatives thereof.

In certain embodiments, the pyrazole compounds have a structure of Formula (I).

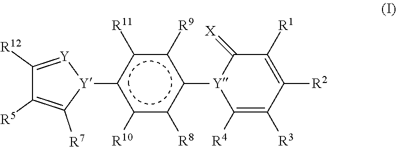

or pharmaceutically acceptable salts and prodrugs thereof wherein,
X is O, S or NR; each of Y, Y′, and Y″ is O, S, or N; R¹, R², R³ and R⁴ are independently H, halogens, alkyl or alkoxy; R⁵ is H, halogens, alkyl, —CH₂N(R⁶)₂, —CH₂OR⁶, —COR⁶, —COOR⁶, —CON(R⁶)₂, —CONHR⁶, —SO₂R⁶, —NHCOR⁶, aryl, or a heterocyclic ring; R⁶ is H, alkyl, aryl, or hetero-aryl; R⁷ is halogen, acetyl, carbonyl, nitro, cyano, —NR₃⁺, trifluoromethylsulfonyl, sulfonyl, or trihaloalkyl; R⁸, R⁹, R¹⁰, R¹¹, and R¹² are independently H, halogens, alkyl or alkoxy; and R is H or alkyl.

In certain embodiments of Formula (I), R⁸, R⁹, R¹⁰, R¹¹, and R¹² are independently H or alkyl (e.g., C₁-C₅ alkyl, C₁-C₄ alkyl, C₁-C₃ alkyl, or C₁-C₂ alkyl).

In certain embodiments, the pyrzaole compounds have a structure of Formula (II).

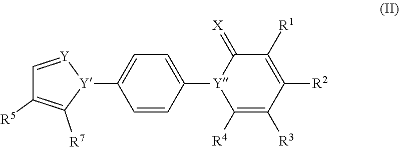

or pharmaceutically acceptable salts and prodrugs thereof wherein,
X is O, S or NR; each of Y, Y′, and Y″ is O, S, or N; R is H or alkyl; R¹, R², R³ and R⁴ are independently H, halogens, alkyl or alkoxy; R⁵ is H, halogens, alkyl, —CH₂N(R⁶)₂, —CH₂OR⁶, —COR⁶, —COOR⁶, —CON(R⁶)₂, —SO₂R⁶, —NHCOR⁶, aryl, or a heterocyclic ring; R⁶ is H, alkyl, aryl, or hetero-aryl; R⁷ is NHR, OR, alkyl, aryl. In some embodiments of Formula (II), R⁷ is trihaloalkyl.

In certain embodiments of Formula (II), R⁵ is —CH₂N(R⁶)₂, —CH₂OR⁶, —COR⁶, —COOR⁶, —CON(R⁶)₂, —SO₂R⁶, —NHCOR⁶, aryl, or heterocyclic ring. In certain embodiments of Formula (II), R⁵ is alkyl, —CH₂N(R⁶)₂, —CON(R⁶)₂, or heterocyclic ring.

In certain embodiments of Formula (II), R⁷ is trifluoroalkyl, trichloroalkyl, tribromoalkyl, or triiodoalkyl where the alkyl is a C₁-C₅ alkyl, C₁-C₄ alkyl, C₁-C₃ alkyl, or C₁-C₂ alkyl, such as methyl.

In certain embodiments, the pyrazole compounds have a structure of Formula (III).

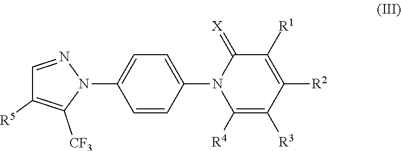

or pharmaceutically acceptable salts and prodrugs thereof wherein,
X is O, S or NR; R is H or alkyl; R¹, R², R³ and R⁴ are independently H, halogens, alkyl or alkoxy; R⁵ is H, halogens, alkyl, —COR⁶, —COOR⁶, —CONHR⁶, —SO₂R⁶, —NHCOR⁶, aryl, or a heterocyclic ring; and R⁶ is H, alkyl, aryl, or hetero-aryl.

In certain embodiments, the pyrazole compounds have a structure of Formula (IV) or (V).

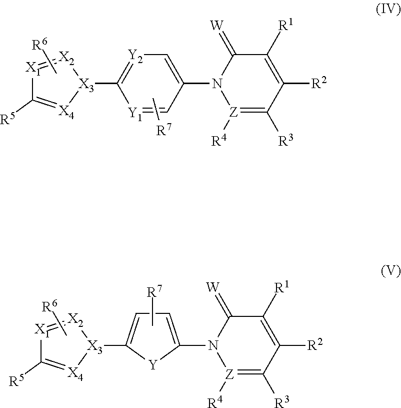

or pharmaceutically acceptable salts and prodrugs thereof wherein,
W is absent, O, S or NR; X₁, X₂, X₃, and X₄ are independently C, O, S, or NR; Y, Y₁, and Y₂ are independently C, O, S or N; Z is C or N; R is H or alkyl; R¹, R², R³, and R⁴ are independently H, hydroxyl, halogens, alkyl, or alkoxy; R⁵ is H, halogens, alkyl, —CH₂N(R⁸)₂, —CH₂OR⁸, —COR⁸, —COOR⁸, —CON(R⁸)₂, —SO₂R⁸, —NHCOR⁸, aryl, or heterocyclic ring; R⁶ and R⁸ are independently H, NHR, OR, alkyl, aryl, or hetero-aryl; and R⁷ is H, halogens, alkyl, or alkoxy.

In certain embodiments, the pyrzaole compounds have a structure of Formula (VI).

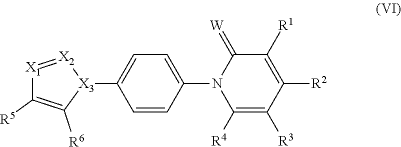

or pharmaceutically acceptable salts and prodrugs thereof wherein,
W is O, S, or NR; X₁, X₂, and X₃ are independently C, O, S, or NR; R is H or alkyl; R¹, R², R³ and R⁴ are independently H, hydroxyl, halogens, alkyl, or alkoxy; R⁵ is H, halogens, alkyl, —CH₂N(R⁸)₂, —CH₂OR⁸, —COR⁸, —COOR⁸, —CON(R⁸)₂, —SO₂R⁸, —NHCOR⁸, aryl, or a heterocyclic ring; R⁶ and R⁸ are independently H, NHR, OR, alkyl, aryl, or hetero-aryl.

In certain embodiments of Formula (VI), W is O or NR. In certain embodiments of Formula (VI), W is O. In certain embodiments of Formula (VI), R¹, R², R³ and R⁴ are independently H, hydroxyl, or halogens. In certain embodiments of Formula (VI), X₁, X₂, and X₃ are independently C, O, or NR. In certain embodiments of Formula (VI), R⁵ is —CH₂N(R⁸)₂, —CH₂OR⁸, —COR⁸, —COOR⁸, —CON(R⁸)₂, —SO₂R⁸, —NHCOR⁸, aryl, or heterocyclic ring; R⁸ is H, NHR, OR, alkyl, aryl, or hetero-aryl.

In certain embodiments of Formula (VI), R⁵ is —CH₂N(R⁸)₂, —CON(R⁸)₂, or heterocyclic ring; R⁸ is H or alkyl. In certain embodiments of Formula (VI), R⁶ is hetero-alkyl, such as trihaloalkyl, for example trifluoroalkyl, trichloroalkyl, tribromoalkyl, or triiodoalkyl where the alkyl is a C₁-C₅ alkyl, C₁-C₄ alkyl, C₁-C₃ alkyl, or C₁-C₂ alkyl, such as methyl. In certain embodiments, the pyrazole compound is any one of compounds 1-79 in Tables 1, 2, and 3.

TABLE 1
Compounds 1-20

| | |
|---|---|
| 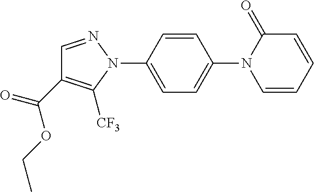 [SMILES: CCOC(=O)c1cnn(-c2ccc(-n3ccccc3=O)cc2)c1C(F)(F)F] | 1 |
| 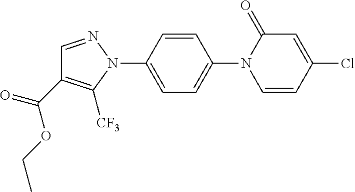 [SMILES: CCOC(=O)c1cnn(-c2ccc(-n3ccc(Cl)cc3=O)cc2)c1C(F)(F)F] | 2 |
| 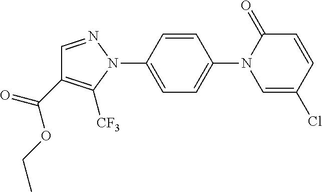 [SMILES: CCOC(=O)c1cnn(-c2ccc(-n3cc(Cl)ccc3=O)cc2)c1C(F)(F)F] | 3 |
| 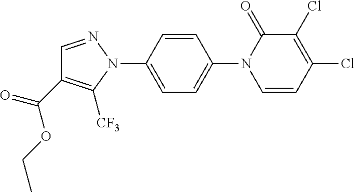 [SMILES: CCOC(=O)c1cnn(-c2ccc(-n3ccc(Cl)c(Cl)c3=O)cc2)c1C(F)(F)F] | 4 |
| 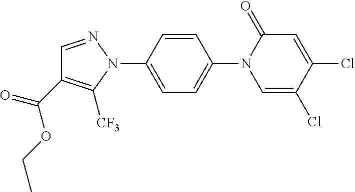 [SMILES: CCOC(=O)c1cnn(-c2ccc(-n3cc(Cl)c(Cl)cc3=O)cc2)c1C(F)(F)F] | 5 |
| 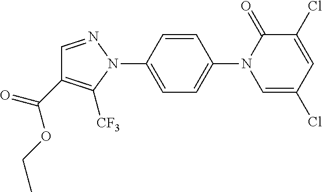 [SMILES: CCOC(=O)c1cnn(-c2ccc(-n3cc(Cl)cc(Cl)c3=O)cc2)c1C(F)(F)F] | 6 |
| 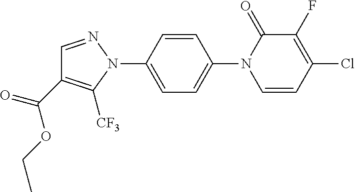 [SMILES: CCOC(=O)c1cnn(-c2ccc(-n3ccc(Cl)c(F)c3=O)cc2)c1C(F)(F)F] | 7 |
| 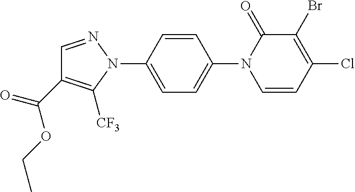 [SMILES: CCOC(=O)c1cnn(-c2ccc(-n3ccc(Cl)c(Br)c3=O)cc2)c1C(F)(F)F] | 8 |
| 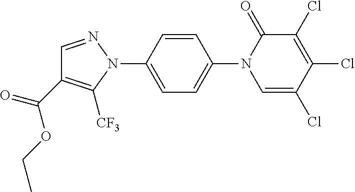 [SMILES: CCOC(=O)c1cnn(-c2ccc(-n3cc(Cl)c(Cl)c(Cl)c3=O)cc2)c1C(F)(F)F] | 9 |
| 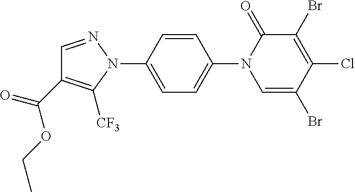 [SMILES: CCOC(=O)c1cnn(-c2ccc(-n3cc(Br)c(Cl)c(Br)c3=O)cc2)c1C(F)(F)F] | 10 |
| 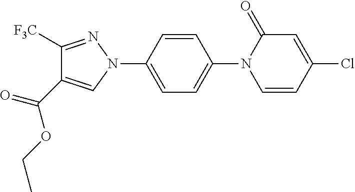 [SMILES: CCOC(=O)c1cn(-c2ccc(-n3ccc(Cl)cc3=O)cc2)nc1C(F)(F)F] | 11 |
| 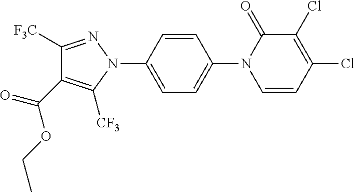 [SMILES: CCOC(=O)c1c(C(F)(F)F)nn(-c2ccc(-n3ccc(Cl)c(Cl)c3=O)cc2)c1C(F)(F)F] | 12 |
| 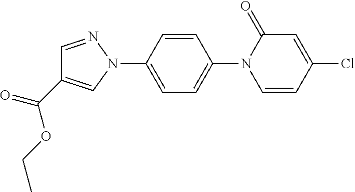 [SMILES: CCOC(=O)c1cnn(-c2ccc(-n3ccc(Cl)cc3=O)cc2)c1] | 13 |
| 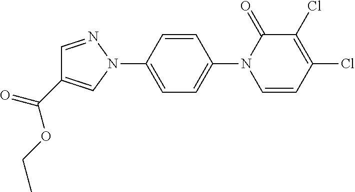 [SMILES: CCOC(=O)c1cnn(-c2ccc(-n3ccc(Cl)c(Cl)c3=O)cc2)c1] | 14 |
| 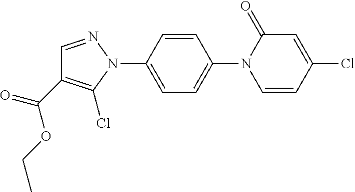 [SMILES: CCOC(=O)c1cnn(-c2ccc(-n3ccc(Cl)cc3=O)cc2)c1Cl] | 15 |
| 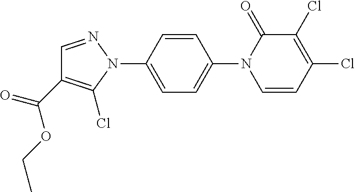 [SMILES: CCOC(=O)c1cnn(-c2ccc(-n3ccc(Cl)c(Cl)c3=O)cc2)c1Cl] | 16 |
| 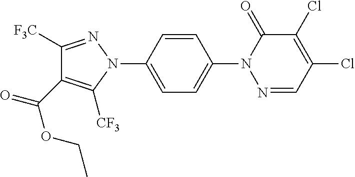 [SMILES: CCOC(=O)c1c(C(F)(F)F)nn(-c2ccc(-n3ncc(Cl)c(Cl)c3=O)cc2)c1C(F)(F)F] | 17 |
| 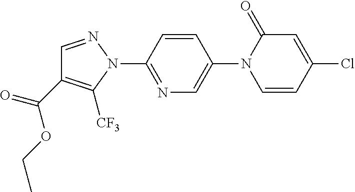 [SMILES: CCOC(=O)c1cnn(-c2ccc(-n3ccc(Cl)cc3=O)cn2)c1C(F)(F)F] | 18 |
| 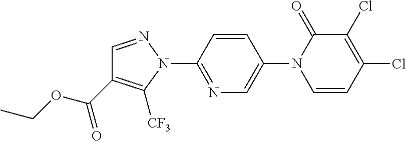 [SMILES: CCOC(=O)c1cnn(-c2ccc(-n3ccc(Cl)c(Cl)c3=O)cn2)c1C(F)(F)F] | 19 |
| 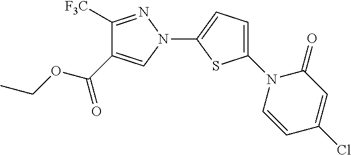 [SMILES: CCOC(=O)c1cn(-c2ccc(-n3ccc(Cl)cc3=O)s2)nc1C(F)(F)F] | 20 |

TABLE 2
Compounds 21-65

| | |
|---|---|
| 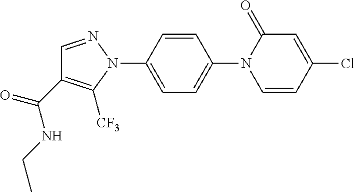 [SMILES: CCNC(=O)c1cnn(-c2ccc(-n3ccc(Cl)cc3=O)cc2)c1C(F)(F)F] | 21 |
| 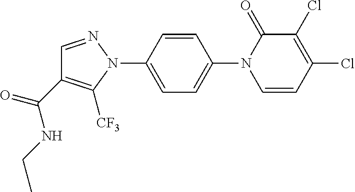 [SMILES: CCNC(=O)c1cnn(-c2ccc(-n3ccc(Cl)c(Cl)c3=O)cc2)c1C(F)(F)F] | 22 |
| 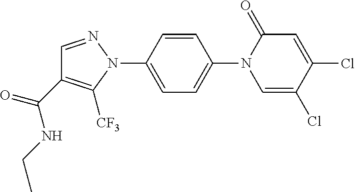 [SMILES: CCNC(=O)c1cnn(-c2ccc(-n3cc(Cl)c(Cl)cc3=O)cc2)c1C(F)(F)F] | 23 |
| 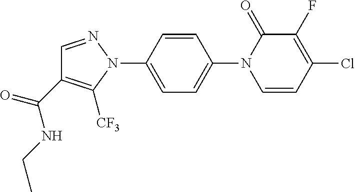 [SMILES: CCNC(=O)c1cnn(-c2ccc(-n3ccc(Cl)c(F)c3=O)cc2)c1C(F)(F)F] | 24 |
| 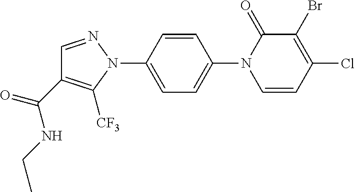 [SMILES: CCNC(=O)c1cnn(-c2ccc(-n3ccc(Cl)c(Br)c3=O)cc2)c1C(F)(F)F] | 25 |
| 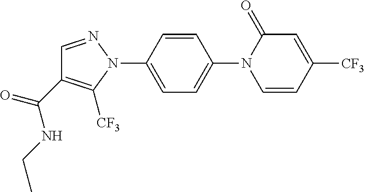 [SMILES: CCNC(=O)c1cnn(-c2ccc(-n3ccc(C(F)(F)F)cc3=O)cc2)c1C(F)(F)F] | 26 |
| 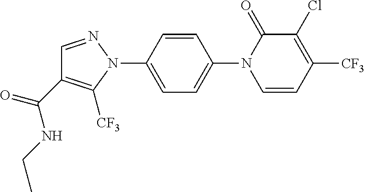 [SMILES: CCNC(=O)c1cnn(-c2ccc(-n3ccc(C(F)(F)F)c(Cl)c3=O)cc2)c1C(F)(F)F] | 27 |
| 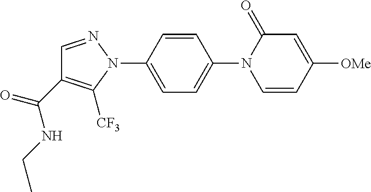 [SMILES: CCNC(=O)c1cnn(-c2ccc(-n3ccc(OC)cc3=O)cc2)c1C(F)(F)F] | 28 |
| 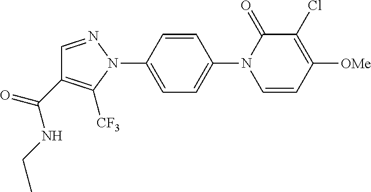 [SMILES: CCNC(=O)c1cnn(-c2ccc(-n3ccc(OC)c(Cl)c3=O)cc2)c1C(F)(F)F] | 29 |
| 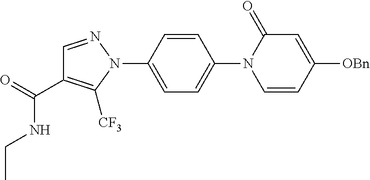 [SMILES: CCNC(=O)c1cnn(-c2ccc(-n3ccc(OCc4ccccc4)cc3=O)cc2)c1C(F)(F)F] | 30 |
| 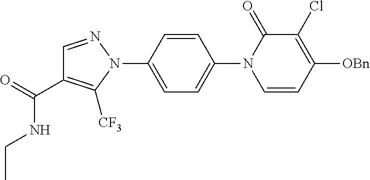 [SMILES: CCNC(=O)c1cnn(-c2ccc(-n3ccc(OCc4ccccc4)c(Cl)c3=O)cc2)c1C(F)(F)F] | 31 |
| 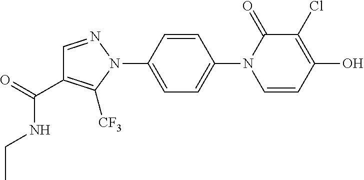 [SMILES: CCNC(=O)c1cnn(-c2ccc(-n3ccc(O)c(Cl)c3=O)cc2)c1C(F)(F)F] | 32 |
| 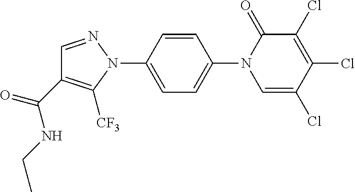 [SMILES: CCNC(=O)c1cnn(-c2ccc(-n3cc(Cl)c(Cl)c(Cl)c3=O)cc2)c1C(F)(F)F] | 33 |
| 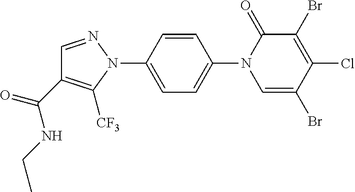 [SMILES: CCNC(=O)c1cnn(-c2ccc(-n3cc(Br)c(Cl)c(Br)c3=O)cc2)c1C(F)(F)F] | 34 |
| 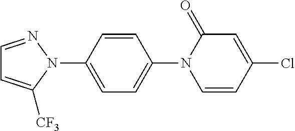 [SMILES: O=c1cc(Cl)ccn1-c1ccc(-n2nccc2C(F)(F)F)cc1] | 35 |
| 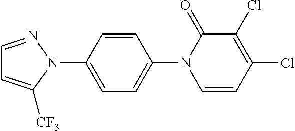 [SMILES: O=c1c(Cl)c(Cl)ccn1-c1ccc(-n2nccc2C(F)(F)F)cc1] | 36 |
| 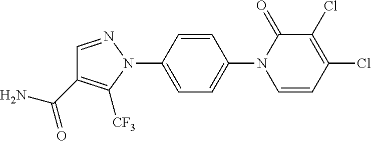 [SMILES: NC(=O)c1cnn(-c2ccc(-n3ccc(Cl)c(Cl)c3=O)cc2)c1C(F)(F)F] | 37 |
| 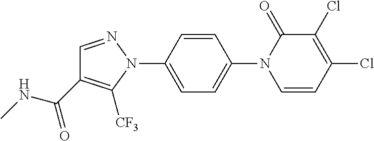 [SMILES: CNC(=O)c1cnn(-c2ccc(-n3ccc(Cl)c(Cl)c3=O)cc2)c1C(F)(F)F] | 38 |
| 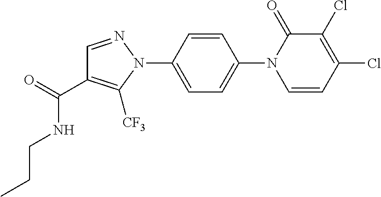 [SMILES: CCCNC(=O)c1cnn(-c2ccc(-n3ccc(Cl)c(Cl)c3=O)cc2)c1C(F)(F)F] | 39 |
| 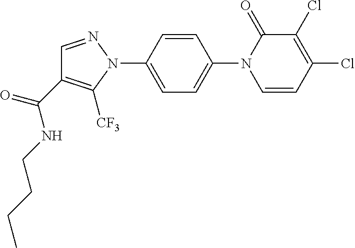 [SMILES: CCCCNC(=O)c1cnn(-c2ccc(-n3ccc(Cl)c(Cl)c3=O)cc2)c1C(F)(F)F] | 40 |
| 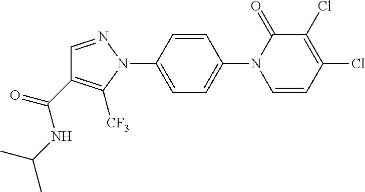 [SMILES: CC(C)NC(=O)c1cnn(-c2ccc(-n3ccc(Cl)c(Cl)c3=O)cc2)c1C(F)(F)F] | 41 |
| 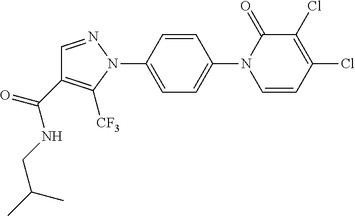 [SMILES: CC(C)CNC(=O)c1cnn(-c2ccc(-n3ccc(Cl)c(Cl)c3=O)cc2)c1C(F)(F)F] | 42 |
| 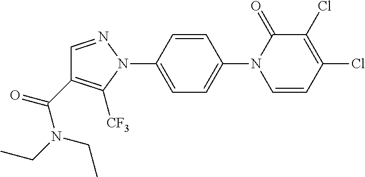 [SMILES: CCN(CC)C(=O)c1cnn(-c2ccc(-n3ccc(Cl)c(Cl)c3=O)cc2)c1C(F)(F)F] | 43 |
| 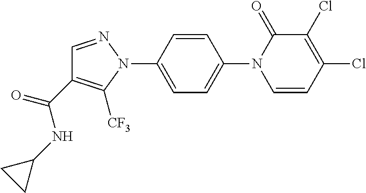 [SMILES: O=C(NC1CC1)c1cnn(-c2ccc(-n3ccc(Cl)c(Cl)c3=O)cc2)c1C(F)(F)F] | 44 |
| 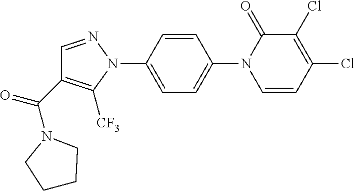 [SMILES: O=C(c1cnn(-c2ccc(-n3ccc(Cl)c(Cl)c3=O)cc2)c1C(F)(F)F)N1CCCC1] | 45 |
| 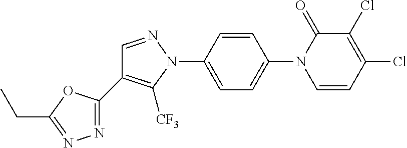 [SMILES: CCc1nnc(-c2cnn(-c3ccc(-n4ccc(Cl)c(Cl)c4=O)cc3)c2C(F)(F)F)o1] | 46 |
| 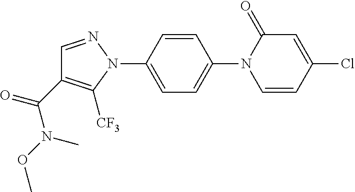 [SMILES: CON(C)C(=O)c1cnn(-c2ccc(-n3ccc(Cl)cc3=O)cc2)c1C(F)(F)F] | 47 |
| 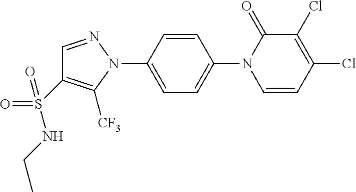 [SMILES: CCNS(=O)(=O)c1cnn(-c2ccc(-n3ccc(Cl)c(Cl)c3=O)cc2)c1C(F)(F)F] | 48 |
| 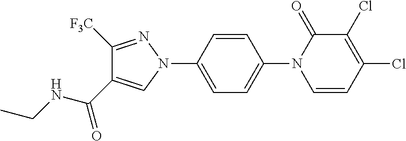 [SMILES: CCNC(=O)c1cn(-c2ccc(-n3ccc(Cl)c(Cl)c3=O)cc2)nc1C(F)(F)F] | 49 |
| 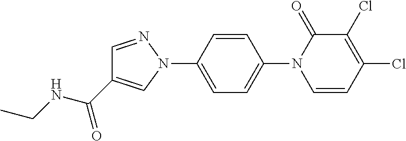 [SMILES: CCNC(=O)c1cnn(-c2ccc(-n3ccc(Cl)c(Cl)c3=O)cc2)c1] | 50 |
| 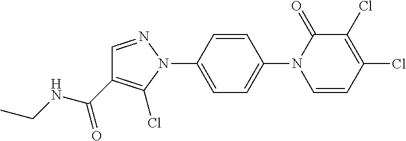 [SMILES: CCNC(=O)c1cnn(-c2ccc(-n3ccc(Cl)c(Cl)c3=O)cc2)c1Cl] | 51 |
| 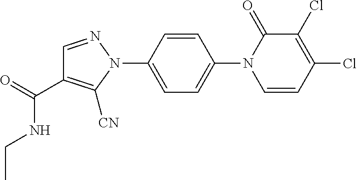 [SMILES: CCNC(=O)c1cnn(-c2ccc(-n3ccc(Cl)c(Cl)c3=O)cc2)c1C#N] | 52 |
| 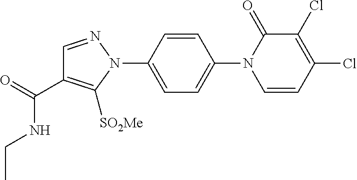 [SMILES: CCNC(=O)c1cnn(-c2ccc(-n3ccc(Cl)c(Cl)c3=O)cc2)c1S(C)(=O)=O] | 53 |
| 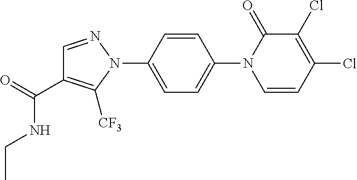 [SMILES: CCNC(=O)c1cnn(-c2ccc(-n3ccc(Cl)c(Cl)c3=O)cc2)c1C(F)(F)F] | 54 |
| 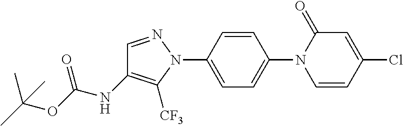 [SMILES: CC(C)(C)OC(=O)Nc1cnn(-c2ccc(-n3ccc(Cl)cc3=O)cc2)c1C(F)(F)F] | 55 |
| 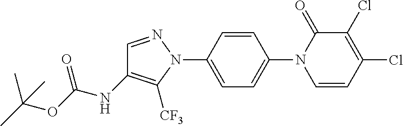 [SMILES: CC(C)(C)OC(=O)Nc1cnn(-c2ccc(-n3ccc(Cl)c(Cl)c3=O)cc2)c1C(F)(F)F] | 56 |
| 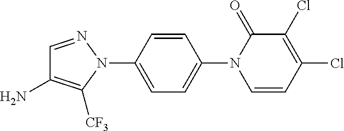 [SMILES: Nc1cnn(-c2ccc(-n3ccc(Cl)c(Cl)c3=O)cc2)c1C(F)(F)F] | 57 |
| 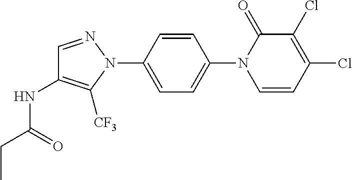 [SMILES: CCC(=O)Nc1cnn(-c2ccc(-n3ccc(Cl)c(Cl)c3=O)cc2)c1C(F)(F)F] | 58 |
| 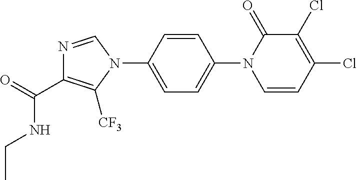 [SMILES: CCNC(=O)c1ncn(-c2ccc(-n3ccc(Cl)c(Cl)c3=O)cc2)c1C(F)(F)F] | 59 |
| 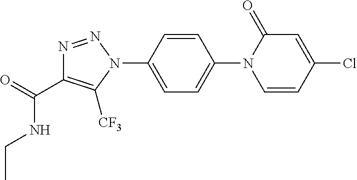 [SMILES: CCNC(=O)c1nnn(-c2ccc(-n3ccc(Cl)cc3=O)cc2)c1C(F)(F)F] | 60 |
| 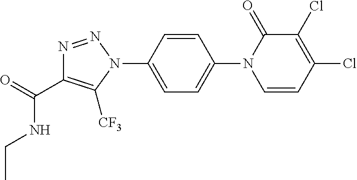 [SMILES: CCNC(=O)c1nnn(-c2ccc(-n3ccc(Cl)c(Cl)c3=O)cc2)c1C(F)(F)F] | 61 |
| 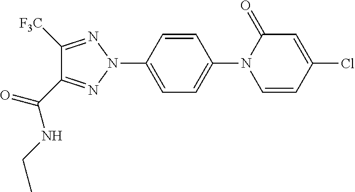 [SMILES: CCNC(=O)c1nn(-c2ccc(-n3ccc(Cl)cc3=O)cc2)nc1C(F)(F)F] | 62 |
| 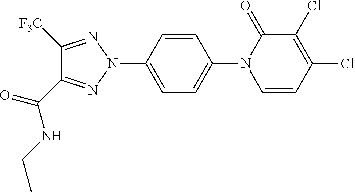 [SMILES: CCNC(=O)c1nn(-c2ccc(-n3ccc(Cl)c(Cl)c3=O)cc2)nc1C(F)(F)F] | 63 |
| 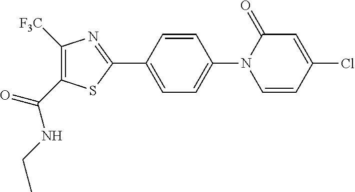 [SMILES: CCNC(=O)c1sc(-c2ccc(-n3ccc(Cl)cc3=O)cc2)nc1C(F)(F)F] | 64 |
| 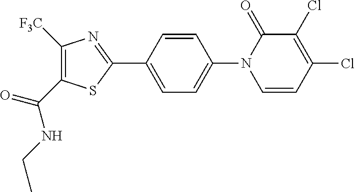 [SMILES: CCNC(=O)c1sc(-c2ccc(-n3ccc(Cl)c(Cl)c3=O)cc2)nc1C(F)(F)F] | 65 |

TABLE 3
Compounds 66-79

| | |
|---|---|
| 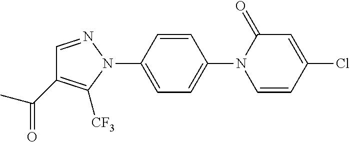 [SMILES: CC(=O)c1cnn(-c2ccc(-n3ccc(Cl)cc3=O)cc2)c1C(F)(F)F] | 66 |
| 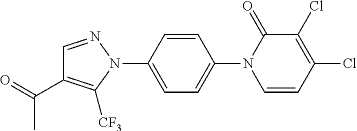 [SMILES: CC(=O)c1cnn(-c2ccc(-n3ccc(Cl)c(Cl)c3=O)cc2)c1C(F)(F)F] | 67 |
| 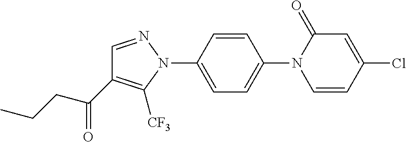 [SMILES: CCCC(=O)c1cnn(-c2ccc(-n3ccc(Cl)cc3=O)cc2)c1C(F)(F)F] | 68 |
| 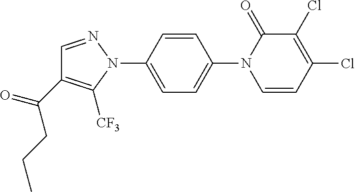 [SMILES: CCCC(=O)c1cnn(-c2ccc(-n3ccc(Cl)c(Cl)c3=O)cc2)c1C(F)(F)F] | 69 |
| 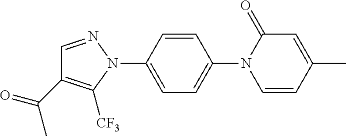 [SMILES: CC(=O)c1cnn(-c2ccc(-n3ccc(C)cc3=O)cc2)c1C(F)(F)F] | 70 |
| 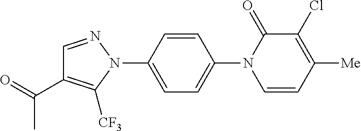 [SMILES: CC(=O)c1cnn(-c2ccc(-n3ccc(C)c(Cl)c3=O)cc2)c1C(F)(F)F] | 71 |
| 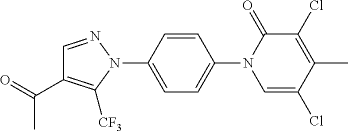 [SMILES: CC(=O)c1cnn(-c2ccc(-n3cc(Cl)c(C)c(Cl)c3=O)cc2)c1C(F)(F)F] | 72 |
| 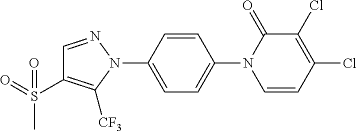 [SMILES: CS(=O)(=O)c1cnn(-c2ccc(-n3ccc(Cl)c(Cl)c3=O)cc2)c1C(F)(F)F] | 73 |
| 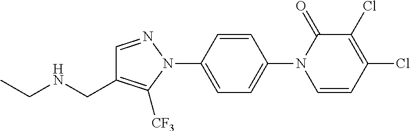 [SMILES: CCNCc1cnn(-c2ccc(-n3ccc(Cl)c(Cl)c3=O)cc2)c1C(F)(F)F] | 74 |
| 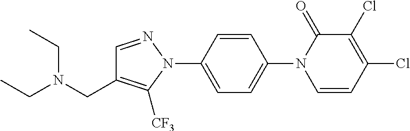 [SMILES: CCN(CC)Cc1cnn(-c2ccc(-n3ccc(Cl)c(Cl)c3=O)cc2)c1C(F)(F)F] | 75 |
| 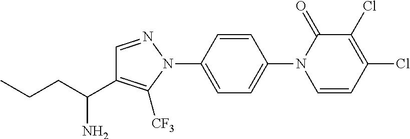 [SMILES: CCCC(N)c1cnn(-c2ccc(-n3ccc(Cl)c(Cl)c3=O)cc2)c1C(F)(F)F] | 76 |
| 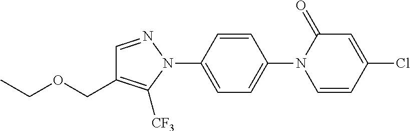 [SMILES: CCOCc1cnn(-c2ccc(-n3ccc(Cl)cc3=O)cc2)c1C(F)(F)F] | 77 |
| 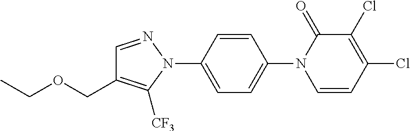 [SMILES: CCOCc1cnn(-c2ccc(-n3ccc(Cl)c(Cl)c3=O)cc2)c1C(F)(F)F] | 78 |
| 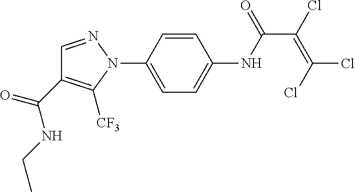 [SMILES: CCNC(=O)c1cnn(-c2ccc(NC(=O)C(Cl)=C(Cl)Cl)cc2)c1C(F)(F)F] | 79 |

### B. Pharmaceutical Compositions

In certain embodiments, this disclosure contemplates pharmaceutical compositions comprising compounds disclosed herein in a pharmaceutically acceptable form. In certain embodiments, this disclosure contemplates pharmaceutical compositions comprising compounds disclosed herein in a pharmaceutically acceptable form and pharmaceutically acceptable excipient. In certain embodiments, this disclosure contemplates the production of a medicament comprising compounds disclosed herein and uses for methods disclosed herein.

As used herein, a “pharmaceutically acceptable form” of a disclosed compound includes, but is not limited to, pharmaceutically acceptable salts, hydrates, solvates, isomers, prodrugs, and isotopically labeled derivatives of disclosed compounds. In one embodiment, a “pharmaceutically acceptable form” includes, but is not limited to, pharmaceutically acceptable salts of disclosed compounds.

In certain embodiments, the pharmaceutically acceptable form is a pharmaceutically acceptable salt. As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, naphthalene-m,n-bissulfonates, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, naphthalene-m,n-bissulfonic acids and the like.

Pharmaceutically acceptable salts derived from appropriate bases include alkali metal, alkaline earth metal, ammonium and quaternary ammonium, e.g., N⁺(R)₄, salts. Representative alkali or alkaline earth metal salts include sodium, lithium, potassium, calcium, magnesium, iron, zinc, copper, manganese, aluminum, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate. Organic bases from which salts can be derived include, for example, primary, secondary, and tertiary amines, substituted amines including naturally occurring substituted amines, cyclic amines, basic ion exchange resins, and the like, such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

In certain embodiments, the pharmaceutically acceptable form is a solvate (e.g., a hydrate). As used herein, the term “solvate” refers to compounds that further include a stoichiometric or non-stoichiometric amount of solvent bound by non-covalent intermolecular forces. The solvate can be of a disclosed compound or a pharmaceutically acceptable salt thereof. Where the solvent is water, the solvate is a “hydrate”. Pharmaceutically acceptable solvates and hydrates are complexes that, for example, can include 1 to about 100, or 1 to about 10, or one to about 2, about 3 or about 4, solvent or water molecules. It will be understood that the term “compound” as used herein encompasses the compound and solvates of the compound, as well as mixtures thereof.

Pharmaceutical compositions typically comprise an effective amount of compounds and a suitable pharmaceutically acceptable carrier. The preparations can be prepared in a manner known per se, which usually involves mixing the compounds according to the disclosure with the one or more pharmaceutically acceptable carriers, and, if desired, in combination with other pharmaceutical active compounds, when necessary under aseptic conditions.

In certain embodiments, the disclosure relates to pharmaceutical compositions comprising compounds disclosed herein and a pharmaceutically acceptable excipient. In certain embodiments, the composition is a pill, tablet, gel, granule, or in a capsule or the composition is an aqueous phosphate buffer, e.g., isotonic solution with a pH between 6 and 8. In certain embodiments, the pharmaceutically acceptable excipient is selected from a filler, glidant, binder, disintegrant, lubricant, and saccharide.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents solvents or vehicles include water, ethanol, polyols (propylene glycol, polyethylene glycol, glycerol, and the like), suitable mixtures thereof, vegetable (such as olive oil, sesame oil and viscoleo) and injectable organic esters such as ethyl oleate.

Prevention of the action of microorganisms may be controlled by addition of any of various antibacterial and antifungal agents, example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

Solid dosage forms for oral administration include capsules, tablets, pills, powders and granules. In such solid dosage forms, the compounds may be admixed with at least one inert customary excipient (or carrier) such as sodium citrate or dicalcium phosphate or: (a) fillers or extenders, as for example, starches, lactose, sucrose, glucose, mannitol and silicic acid, (b) binders, as for example, carboxymethylcellulose, alginates, gelatin, polyvinylpyrrolidone, sucrose, and acacia, (c) humectants, as for example, glycerol (d) disintegrating agents, as for example, agar-agar, calcium carbonate, potato or tapioca starch, alginic acid, certain complex silicates, and sodium carbonate, (e) solution retarders, as for example paraffin, (f) absorption accelerators, as for example, quaternary ammonium compounds, (g) wetting agents, as for example cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Liquid dosage forms for oral administration include pharmaceutically acceptable emulsions, solutions, suspensions, syrups, and elixirs. In addition to the compounds, the liquid dosage forms may contain inert diluents commonly used in the art, such as water or other solvents, solubilizing agents and emulsifiers, for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, viscoleo, castor oil and sesame oil, glycerol, tetrahydrofurfuryl alcohol, polyethyleneglycols and fatty acid esters of sorbitan or mixtures of these substances, and the like.

### 1. Parenteral Formulations

The compounds described herein can be formulated for parenteral administration.

For example, parenteral administration may include administration to a patient intravenously, intradermally, intraarterially, intraperitoneally, intralesionally, intracranially, intraarticularly, intraprostatically, intrapleurally, intratracheally, intravitreally, intratumorally, intramuscularly, subcutaneously, subconjunctivally, intravesicularly, intrapericardially, intraumbilically, by injection, and by infusion.

Parenteral formulations can be prepared as aqueous compositions using techniques known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, using a coating, such as lecithin, by the maintenance of the required particle size in the case of dispersion and/or by the use of surfactants. In many cases, it will be preferable to include isotonic agents, for example, sugars or sodium chloride.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, viscosity modifying agents, and combination thereof.

Suitable surfactants may be anionic, cationic, amphoteric or nonionic surface-active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-beta-alanine, sodium N-lauryl-beta-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers.

Water-soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

### (a) Controlled Release Formulations

The parenteral formulations described herein can be formulated for controlled release including immediate release, delayed release, extended release, pulsatile release, and combinations thereof.

### 1. Nano- and Microparticles

For parenteral administration, the one or more compounds, and optional one or more additional active agents, can be incorporated into microparticles, nanoparticles, or combinations thereof that provide controlled release of the compounds and/or one or more additional active agents. In embodiments wherein the formulations contain two or more drugs, the drugs can be formulated for the same type of controlled release (e.g., delayed, extended, immediate, or pulsatile) or the drugs can be independently formulated for different types of release (e.g., immediate and delayed, immediate and extended, delayed and extended, delayed and pulsatile, etc.).

For example, the compounds and/or one or more additional active agents can be incorporated into polymeric microparticles, which provide controlled release of the drug(s). Release of the drug(s) is controlled by diffusion of the drug(s) out of the microparticles and/or degradation of the polymeric particles by hydrolysis and/or enzymatic degradation. Suitable polymers include ethylcellulose and other natural or synthetic cellulose derivatives.

Polymers, which are slowly soluble and form a gel in an aqueous environment, such as hydroxypropyl methylcellulose or polyethylene oxide, can also be suitable as materials for drug containing microparticles. Other polymers include, but are not limited to, polyanhydrides, poly (ester anhydrides), polyhydroxy acids, such as polylactide (PLA), polyglycolide (PGA), poly(lactide-co-glycolide) (PLGA), poly-3-hydroxybutyrate (PHB) and copolymers thereof, poly-4-hydroxybutyrate (P4HB) and copolymers thereof, polycaprolactone and copolymers thereof, and combinations thereof.

Alternatively, the drug(s) can be incorporated into microparticles prepared from materials, which are insoluble in aqueous solution or slowly soluble in aqueous solution but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, wax-like substances and mixtures thereof. Suitable fats and fatty substances include fatty alcohols (such as lauryl, myristyl stearyl, cetyl or cetostearyl alcohol), fatty acids and derivatives, including but not limited to fatty acid esters, fatty acid glycerides (mono-, di- and tri-glycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes. Specific examples of waxes include beeswax, glycowax, castor wax, carnauba wax, paraffins and candelilla wax. As used herein, a wax-like material is defined as any material, which is normally solid at room temperature and has a melting point of from about 30 to 300° C.

In some cases, it may be desirable to alter the rate of water penetration into the microparticles. To this end, rate-controlling (wicking) agents can be formulated along with the fats or waxes listed above. Examples of rate-controlling materials include certain starch derivatives (e.g., waxy maltodextrin and drum dried corn starch), cellulose derivatives (e.g., hydroxypropylmethyl-cellulose, hydroxypropylcellulose, methylcellulose, and carboxymethyl-cellulose), alginic acid, lactose and talc. Additionally, a pharmaceutically acceptable surfactant (for example, lecithin) may be added to facilitate the degradation of such microparticles.

Proteins, which are water insoluble, such as zein, can also be used as materials for the formation of drug containing microparticles. Additionally, proteins, polysaccharides and combinations thereof, which are water-soluble, can be formulated with drug into microparticles and subsequently cross-linked to form an insoluble network. For example, cyclodextrins can be complexed with individual drug molecules and subsequently cross-linked.

### 2. Method of Making Nano- and Microparticles

Encapsulation or incorporation of drug into carrier materials to produce drug-containing microparticles can be achieved through known pharmaceutical formulation techniques. In the case of formulation in fats, waxes or wax-like materials, the carrier material is typically heated above its melting temperature and the drug is added to form a mixture comprising drug particles suspended in the carrier material, drug dissolved in the carrier material, or a mixture thereof. Microparticles can be subsequently formulated through several methods including, but not limited to, the processes of congealing, extrusion, spray chilling or aqueous dispersion. In a preferred process, wax is heated above its melting temperature, drug is added, and the molten wax-drug mixture is congealed under constant stirring as the mixture cools. Alternatively, the molten wax-drug mixture can be extruded and spheronized to form pellets or beads. These processes are known in the art.

For some carrier materials it may be desirable to use a solvent evaporation technique to produce drug-containing microparticles. In this case drug and carrier material are co-dissolved in a mutual solvent and microparticles can subsequently be produced by several techniques including, but not limited to, forming an emulsion in water or other appropriate media, spray drying or by evaporating off the solvent from the bulk solution and milling the resulting material.

In some embodiments, drug in a particulate form is homogeneously dispersed in a water-insoluble or slowly water-soluble material. To minimize the size of the drug particles within the composition, the drug powder itself may be milled to generate fine particles prior to formulation. The process of jet milling, known in the pharmaceutical art, can be used for this purpose. In some embodiments drug in a particulate form is homogeneously dispersed in a wax or wax like substance by heating the wax or wax like substance above its melting point and adding the drug particles while stirring the mixture. In this case a pharmaceutically acceptable surfactant may be added to the mixture to facilitate the dispersion of the drug particles.

The particles can also be coated with one or more modified release coatings. Solid esters of fatty acids, which are hydrolyzed by lipases, can be spray coated onto microparticles or drug particles. Zein is an example of a naturally water-insoluble protein. It can be coated onto drug containing microparticles or drug particles by spray coating or by wet granulation techniques. In addition to naturally water-insoluble materials, some substrates of digestive enzymes can be treated with cross-linking procedures, resulting in the formation of non-soluble networks. Many methods of cross-linking proteins, initiated by both chemical and physical means, have been reported. One of the most common methods to obtain cross-linking is the use of chemical cross-linking agents. Examples of chemical cross-linking agents include aldehydes (glutaraldehyde and formaldehyde), epoxy compounds, carbodiimides, and genipin. In addition to these cross-linking agents, oxidized and native sugars have been used to cross-link gelatin. Cross-linking can also be accomplished using enzymatic means; for example, transglutaminase has been approved as a GRAS substance for cross-linking seafood products. Finally, cross-linking can be initiated by physical means such as thermal treatment, UV irradiation and gamma irradiation.

To produce a coating layer of cross-linked protein surrounding drug containing microparticles or drug particles, a water-soluble protein can be spray coated onto the microparticles and subsequently cross-linked by the one of the methods described above. Alternatively, drug-containing microparticles can be microencapsulated within protein by coacervation-phase separation (for example, by the addition of salts) and subsequently cross-linked. Some suitable proteins for this purpose include gelatin, albumin, casein, and gluten.

Polysaccharides can also be cross-linked to form a water-insoluble network. For many polysaccharides, this can be accomplished by reaction with calcium salts or multivalent cations, which cross-link the main polymer chains. Pectin, alginate, dextran, amylose and guar gum are subject to cross-linking in the presence of multivalent cations. Complexes between oppositely charged polysaccharides can also be formed; pectin and chitosan, for example, can be complexed via electrostatic interactions.

### (b) Injectable/Implantable Formulations

The compounds described herein can be incorporated into injectable/implantable solid or semi-solid implants, such as polymeric implants. In one embodiment, the compounds are incorporated into a polymer that is a liquid or paste at room temperature, but upon contact with aqueous medium, such as physiological fluids, exhibits an increase in viscosity to form a semi-solid or solid material. Exemplary polymers include, but are not limited to, hydroxyalkanoic acid polyesters derived from the copolymerization of at least one unsaturated hydroxy fatty acid copolymerized with hydroxyalkanoic acids. The polymer can be melted, mixed with the active substance and cast or injection molded into a device. Such melt fabrication requires polymers having a melting point that is below the temperature at which the substance to be delivered and polymer degrade or become reactive. The device can also be prepared by solvent casting where the polymer is dissolved in a solvent and the drug dissolved or dispersed in the polymer solution and the solvent is then evaporated. Solvent processes require that the polymer be soluble in organic solvents. Another method is compression molding of a mixed powder of the polymer and the drug or polymer particles loaded with the active agent.

Alternatively, the compounds can be incorporated into a polymer matrix and molded, compressed, or extruded into a device that is a solid at room temperature. For example, the compounds can be incorporated into a biodegradable polymer, such as polyanhydrides, polyhydroalkanoic acids (PHAs), PLA, PGA, PLGA, polycaprolactone, polyesters, polyamides, polyorthoesters, polyphosphazenes, proteins and polysaccharides such as collagen, hyaluronic acid, albumin and gelatin, and combinations thereof and compressed into solid device, such as disks, or extruded into a device, such as rods.

The release of the one or more compounds from the implant can be varied by selection of the polymer, the molecular weight of the polymer, and/or modification of the polymer to increase degradation, such as the formation of pores and/or incorporation of hydrolyzable linkages. Methods for modifying the properties of biodegradable polymers to vary the release profile of the compounds from the implant are well known in the art.

### 2. Enteral Formulations

Suitable oral dosage forms include tablets, capsules, solutions, suspensions, syrups, and lozenges. Tablets can be made using compression or molding techniques well known in the art. Gelatin or non-gelatin capsules can be prepared as hard or soft capsule shells, which can encapsulate liquid, solid, and semi-solid fill materials, using techniques well known in the art.

Formulations may be prepared using a pharmaceutically acceptable carrier. As generally used herein “carrier” includes, but is not limited to, diluents, preservatives, binders, lubricants, disintegrators, swelling agents, fillers, stabilizers, and combinations thereof.

Carrier also includes all components of the coating composition, which may include plasticizers, pigments, colorants, stabilizing agents, and glidants.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

“Diluents”, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

“Binders” are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

“Lubricants” are used to facilitate tablet manufacture. Examples of suitable lubricants include, but are not limited to, magnesium stearate, calcium stearate, stearic acid, glycerol behenate, polyethylene glycol, talc, and mineral oil.

“Disintegrants” are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross-linked polymers, such as cross-linked PVP (Polyplasdone® XL from GAF Chemical Corp).

“Stabilizers” are used to inhibit or retard drug decomposition reactions, which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

### (a) Controlled Release Enteral Formulations

Oral dosage forms, such as capsules, tablets, solutions, and suspensions, can for formulated for controlled release. For example, the one or more compounds and optional one or more additional active agents can be formulated into nanoparticles, microparticles, and combinations thereof, and encapsulated in a soft or hard gelatin or non-gelatin capsule or dispersed in a dispersing medium to form an oral suspension or syrup. The particles can be formed of the drug and a controlled release polymer or matrix. Alternatively, the drug particles can be coated with one or more controlled release coatings prior to incorporation into the finished dosage form.

In another embodiment, the one or more compounds and optional one or more additional active agents are dispersed in a matrix material, which gels or emulsifies upon contact with an aqueous medium, such as physiological fluids. In the case of gels, the matrix swells entrapping the active agents, which are released slowly over time by diffusion and/or degradation of the matrix material. Such matrices can be formulated as tablets or as fill materials for hard and soft capsules.

In still another embodiment, the one or more compounds, and optional one or more additional active agents are formulated into a sold oral dosage form, such as a tablet or capsule, and the solid dosage form is coated with one or more controlled release coatings, such as a delayed release coatings or extended release coatings. The coating or coatings may also contain the compounds and/or additional active agents.

### (1) Extended Release Dosage Forms

The extended release formulations are generally prepared as diffusion or osmotic systems, which are known in the art. A diffusion system typically consists of two types of devices, a reservoir and a matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers such as methyl and ethyl cellulose, hydroxyalkylcelluloses such as hydroxypropyl-cellulose, hydroxypropylmethylcellulose, sodium carboxymethylcellulose, and Carbopol® 934, polyethylene oxides and mixtures thereof. Fatty compounds include, but are not limited to, various waxes such as carnauba wax and glyceryl tristearate and wax-type substances including hydrogenated castor oil or hydrogenated vegetable oil, or mixtures thereof.

In certain preferred embodiments, the plastic material is a pharmaceutically acceptable acrylic polymer, including but not limited to, acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer poly(methyl methacrylate), poly(methacrylic acidxanhydride), polymethacrylate, polyacrylamide, poly(methacrylic acid anhydride), and glycidyl methacrylate copolymers.

In certain preferred embodiments, the acrylic polymer is comprised of one or more ammonio methacrylate copolymers. Ammonio methacrylate copolymers are well known in the art, and are described in NF XVII as fully polymerized copolymers of acrylic and methacrylic acid esters with a low content of quaternary ammonium groups.

In one preferred embodiment, the acrylic polymer is an acrylic resin lacquer such as that which is commercially available from Rohm Pharma under the tradename EUDRAGIT®. In further preferred embodiments, the acrylic polymer comprises a mixture of two acrylic resin lacquers commercially available from Rohm Pharma under the tradenames EUDRAGIT® RL30D and EUDRAGIT® RS30D, respectively. EUDRAGIT® RL30D and EUDRAGIT® RS30D are copolymers of acrylic and methacrylic esters with a low content of quaternary ammonium groups, the molar ratio of ammonium groups to the remaining neutral (meth)acrylic esters being 1:20 in EUDRAGIT® RL30D and 1:40 in EUDRAGIT® RS30D. The mean molecular weight is about 150,000. EUDRAGIT® S-100 and EUDRAGIT® L-100 are also preferred. The code designations RL (high permeability) and RS (low permeability) refer to the permeability properties of these agents. EUDRAGIT® RL/RS mixtures are insoluble in water and in digestive fluids. However, multiparticulate systems formed to include the same are swellable and permeable in aqueous solutions and digestive fluids.

The polymers described above such as EUDRAGIT® RL/RS may be mixed together in any desired ratio in order to ultimately obtain a sustained-release formulation having a desirable dissolution profile. Desirable sustained-release multiparticulate systems may be obtained, for instance, from 100% EUDRAGIT® RL, 50% EUDRAGIT® RL and 50% EUDRAGIT t® RS, and 10% EUDRAGIT® RL and 90% EUDRAGIT® RS. One skilled in the art will recognize that other acrylic polymers may also be used, such as, for example, EUDRAGIT® L.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above can be combined in a final dosage form comprising single or multiple units. Examples of multiple units include, but are not limited to, multilayer tablets and capsules containing tablets, beads, or granules An immediate release portion can be added to the extended release system by means of either applying an immediate release layer on top of the extended release core using a coating or compression process or in a multiple unit system such as a capsule containing extended and immediate release beads.

Extended release tablets containing hydrophilic polymers are prepared by techniques commonly known in the art such as direct compression, wet granulation, or dry granulation. Their formulations usually incorporate polymers, diluents, binders, and lubricants as well as the active pharmaceutical ingredient. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In the congealing method, the drug is mixed with a wax material and either spray-congealed or congealed and screened and processed.

### (2) Delayed Release Dosage Forms

Delayed release formulations can be created by coating a solid dosage form with a polymer film, which is insoluble in the acidic environment of the stomach, and soluble in the neutral environment of the small intestine.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit® (Rohm Pharma; Westerstadt, Germany), including EUDRAGIT® L30D-55 and L100-55 (soluble at pH 5.5 and above), EUDRAGIT® L-100 (soluble at pH 6.0 and above), EUDRAGIT® S (soluble at pH 7.0 and above, as a result of a higher degree of esterification), and EUDRAGITS® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition

### C. Kits

The discloses compounds can be provided as a kit. In certain embodiments, production processes are contemplated which two components, compounds disclosed herein and a pharmaceutical carrier, are provided already in a combined dry form ready to be reconstituted together. In other embodiments, it is contemplated that compounds disclosed herein and a pharmaceutical carrier are admixed to provide a pharmaceutical composition.

Providing a pharmaceutic composition is possible in a one-step process, simply by adding a suitable pharmaceutically acceptable diluent to the composition in a container. In certain embodiments, the container is preferably a syringe for administering the reconstituted pharmaceutical composition after contact with the diluent. In certain embodiments, the coated compounds can be filled into a syringe, and the syringe can then be closed with the stopper. A diluent is used in an amount to achieve the desired end-concentration. The pharmaceutical composition may contain other useful component, such as ions, buffers, excipients, stabilizers, etc.

A “dry” pharmaceutical composition typically has only a residual content of moisture, which may approximately correspond to the moisture content of comparable commercial products, for example, has about 12% moisture as a dry product. Usually, the dry pharmaceutical composition according to the present invention has a residual moisture content preferably below 10% moisture, more preferred below 5% moisture, especially below 1% moisture. The pharmaceutical composition can also have lower moisture content, e.g., 0.1% or even below. In certain embodiments, the pharmaceutical composition is provided in dry in order to prevent degradation and enable storage stability.

A container can be any container suitable for housing (and storing) pharmaceutically compositions such as syringes, vials, tubes, etc. The pharmaceutical composition may then preferably be applied via specific needles of the syringe or via suitable catheters. A typical diluent comprises water for injection, and NaCl (preferably 50 to 150 mM, especially 110 mM), CaCl₂) (preferably 10 to 80 mM, especially 40 mM), sodium acetate (preferably 0 to 50 mM, especially 20 mM) and mannitol (preferably up to 10% w/w, especially 2% w/w). Preferably, the diluent can also include a buffer or buffer system so as to buffer the pH of the reconstituted dry composition, preferably at a pH of 6.2 to 7.5, especially at pH of 6.9 to 7.1.

In certain embodiments, the diluent is provided in a separate container. This can preferably be a syringe. The diluent in the syringe can then easily be applied to the container for reconstitution of the dry compositions. If the container is also a syringe, both syringes can be finished together in a pack. It is therefore preferred to provide the dry compositions in a syringe, which is finished with a diluent syringe with a pharmaceutically acceptable diluent for reconstituting, said dry and stable composition.

In certain embodiments, this disclosure contemplates a kit comprising a pharmaceutical composition disclosed herein and a container with a suitable diluent. Further components of the kit may be instructions for use, administration means, such as syringes, catheters, brushes, etc. (if the compositions are not already provided in the administration means) or other components necessary for use in medical (surgical) practice, such as substitute needles or catheters, extra vials or further wound cover means. In certain embodiments, the kit comprises a syringe housing the dry and stable hemostatic composition and a syringe containing the diluent (or provided to take up the diluent from another diluent container).

The present invention will be further understood by reference to the following non-limiting examples.

### EXAMPLES

### Example 1

### Synthesis of New TRPC3 Inhibitors

Schemes 1 to 31 illustrate compounds and general methods of preparations.

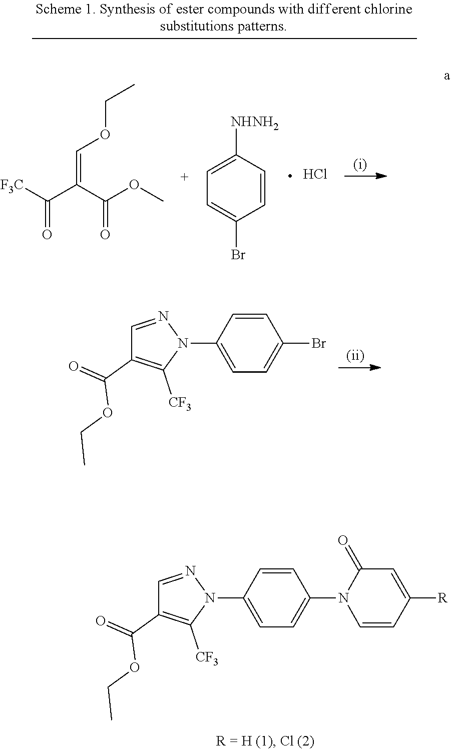

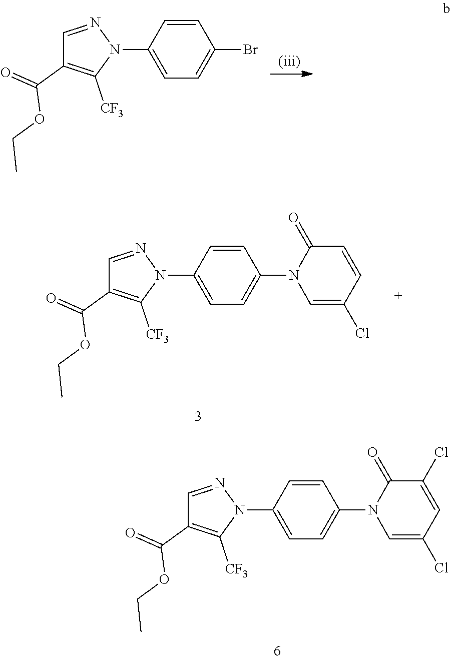 [SMILES: CCOC(=O)c1cnn(-c2ccc(Br)cc2)c1C(F)(F)F ; CCOC(=O)c1cnn(-c2ccc(-n3cc(Cl)ccc3=O)cc2)c1C(F)(F)F ; CCOC(=O)c1cnn(-c2ccc(-n3cc(Cl)cc(Cl)c3=O)cc2)c1C(F)(F)F]

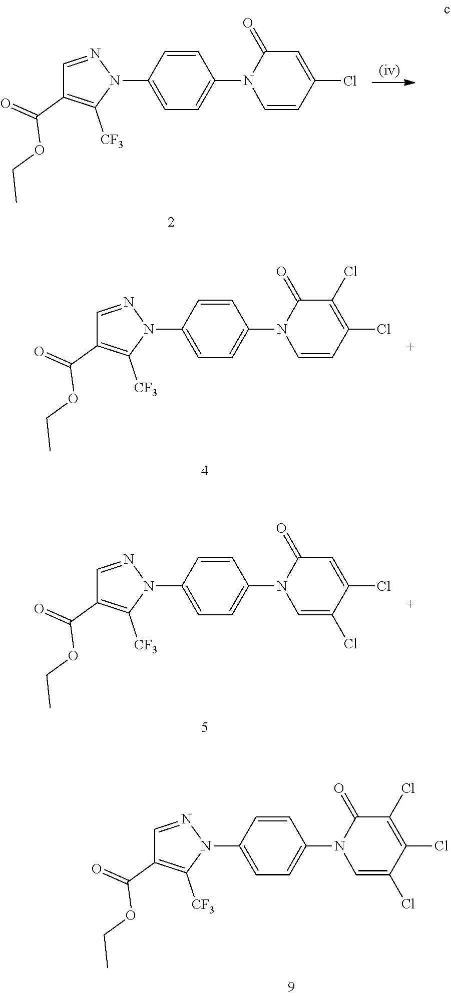 [SMILES: CCOC(=O)c1cnn(-c2ccc(-n3ccc(Cl)cc3=O)cc2)c1C(F)(F)F ; CCOC(=O)c1cnn(-c2ccc(-n3ccc(Cl)c(Cl)c3=O)cc2)c1C(F)(F)F ; CCOC(=O)c1cnn(-c2ccc(-n3cc(Cl)c(Cl)cc3=O)cc2)c1C(F)(F)F ; CCOC(=O)c1cnn(-c2ccc(-n3cc(Cl)c(Cl)c(Cl)c3=O)cc2)c1C(F)(F)F]

Reagents and conditions: (i) EtOH, 60° C., 6 h; (ii) 4-chloropridinone, CuI, DMCDA, K₂CO₃, toluene, reflux, 12 h; (iii) 3,5,6-trichloropyridin-2(1H)-one, CuI, DMCDA, K₂CO₃, toluene, reflux, 12 h; (iv) NCS, DMF, 100° C., 8 h.

### General Procedure for Cyclization Reaction to Form Pyrazole Derivatives.

To a stirred solution of substituted phenylhydrazine hydrochloride (18.74 mmol) in ethanol (20 mL) was added ethoxymethylene compound (12.49 mmol). The mixture was stirred and heated to reflux for 6 hours under argon atmosphere. Then, the solvent was removed under reduced pressure. The yellow solid was stirred in 30 mL of saturated NaHCO₃solution and extracted with ethyl acetate (3×50 mL). The organic layer was separated, dried over anhydrous MgSO₄, filtered and concentrated to dryness under reduced pressure. The yellow oily residue was subjected to flash column chromatography (silica gel, CH₂Cl₂) to afford the pyrazoles as pale yellow solids.

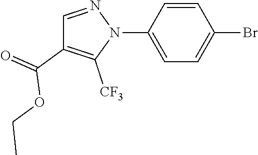 [SMILES: CCOC(=O)c1cnn(-c2ccc(Br)cc2)c1C(F)(F)F]

Ethyl 1-(4-bromophenyl)-5-(trifluoromethyl)-1H-pyrazole-4-carboxylate. 77.2% yield; ¹H NMR (400 MHz, CDCl₃) δ 8.11 (s, 1H), 7.64 (d, J=8.8 Hz, 2H), 7.31 (d, J=8.8 Hz, 2H), 4.37 (q, J=7.2 Hz, 2H), 1.39 (t, J=7.2 Hz, 3H).

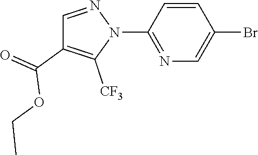 [SMILES: CCOC(=O)c1cnn(-c2ccc(Br)cn2)c1C(F)(F)F]

Ethyl 1-(5-bromopyridin-2-yl)-5-(trifluoromethyl)-1H-pyrazole-4-carboxylate. 81.3% yield; ¹H NMR (400 MHz, CDCl₃) δ 8.62 (d, J=2.3 Hz, 1H), 8.12 (s, 1H), 8.05 (dd, J=8.5 Hz, J₂=2.3 Hz, 1H), 7.56 (d, J=8.5 Hz, 1H), 4.38 (q, J=7.1 Hz, 2H), 1.39 (t, J=7.1 Hz, 3H).

General Procedure for Coupling of Pyrazole Derivatives with Substituted Pyridones.

Pyrazole (3.32 mmol), pyridone (3.32 mmol), CuI (0.66 mmol), trans-N,N′-dimethylcyclohexane-1,2-diamine (0.66 mmol) and K₂CO₃ (6.63 mmol) were mixed together in 20 mL of anhydrous toluene at room temperature under argon. The reaction mixture was stirred and heated to reflux overnight under argon. The reaction was quenched by addition of 100 mL of water and extracted with ethyl acetate (3×50 mL). Organic layer was separated, washed with water, dried over anhydrous MgSO₄, filtered and evaporated to an orange oil which was purified by column chromatography (silica gel, CH₂Cl₂/MeOH=19/1 v/v) to afford products as white solids.

### Chlorination of Pyridone Ring.

Ethyl 1-(4-(4-chloro-2-oxopyridin-1(2H)-yl)phenyl)-5-(trifluoromethyl)-1H-pyrazole-4-carboxylate (2) (1.52 g, 3.69 mmol) and N-chlorosuccinimide (0.54 g, 4.06 mmol) were dissolved in 50 mL of anhydrous DMF at room temperature under argon. The reaction mixture was stirred and heated to 100° C. for 4 hours. The reaction was hydrolyzed by addition of 100 mL of water and extracted with ethyl acetate (3×50 mL). Organic layer was separated, washed with brine, dried over anhydrous MgSO₄, filtered and evaporated to an orange oil which was purified by column chromatography (silica gel, CH₂Cl₂/Acetone=19/1 v/v) to give compounds as white solid products.

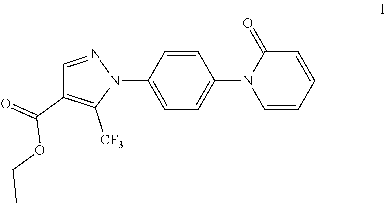 [SMILES: CCOC(=O)c1cnn(-c2ccc(-n3ccccc3=O)cc2)c1C(F)(F)F]

Ethyl 1-(4-(2-oxopyridin-1(2H)-yl)phenyl)-5-(trifluoromethyl)-1H-pyrazole-4-carboxylate (1). 76% yield; ¹H NMR (400 MHz, CDCl₃): δ 8.14 (s, 1H), 7.58 (s, 4H), 7.47-7.43 (m, 1H), 7.38-7.37 (m, 1H), 6.34-6.30 (m, 1H), 4.39 (q, J=7.2 Hz, 2H), 1.40 (t, J=7.2 Hz, 3H). HRMS (ESI): calcd for C₁₈H₁₄F₃N₃O₃ 378.1066 [M+H]⁺, found Purity: 97.2% by HPLC (Rt=3.62 min). HRMS (ESI): calcd for C₁₈H₁₅F₃N₃O₃ 378.1060 [M+H]⁺, found 378.1018.

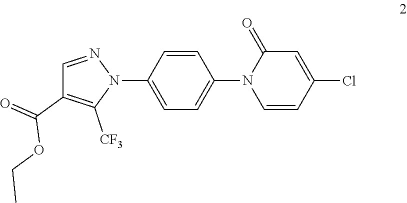 [SMILES: CCOC(=O)c1cnn(-c2ccc(-n3ccc(Cl)cc3=O)cc2)c1C(F)(F)F]

Ethyl 1-(4-(4-chloro-2-oxopyridin-1(2H)-yl)phenyl)-5-(trifluoromethyl)-1H-pyrazole-4-carboxylate (2). 64.7% yield; ¹H NMR (400 MHz, CDCl₃): δ 8.17 (s, 1H), 7.57-7.55 (m, 4H), 7.35 (dd, J=7.6 Hz, J₂=0.4 Hz, 1H), 6.76 (dd, J=2 Hz, J₂=0.4 Hz, 1H), 6.36 (dd, J=7.6 Hz, J₂=2 Hz, 1H), 4.41 (q, J=7.2 Hz, 2H), 1.42 (t, J=7.2 Hz, 3H). HRMS (ESI): calcd for C₁₈H₁₅F₃N₃O₃ 412.0670 [M+H]⁺, found 412.0664. Purity: 99.0% by HPLC (Rt=4.3 min).

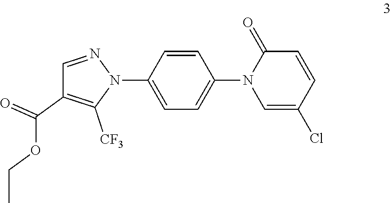 [SMILES: CCOC(=O)c1cnn(-c2ccc(-n3cc(Cl)ccc3=O)cc2)c1C(F)(F)F]

Ethyl 1-(4-(5-chloro-2-oxopyridin-1(2H)-yl)phenyl)-5-(trifluoromethyl)-1H-pyrazole-4-carboxylate (3). 32.4% yield; ¹H NMR (400 MHz, CDCl₃): δ 8.14 (s, 1H); 7.55-7.54 (m, 4H), 7.43 (dd, J=6.8 Hz, J₂=0.8 Hz, 1H), 7.38 (dd, J=9.6 Hz, J₂=2.8 Hz, 1H), 6.66 (dd, J=9.6 Hz, J₂=0.8 Hz, 1H), 4.39 (q, J=7.2 Hz, 2H), 1.39 (t, J=7.2 Hz, 3H). HRMS (ESI): calcd for C₁₈H₁₄C₁F₃N₃O₃ 412.0670 [M+H]⁺, found 412.0676.

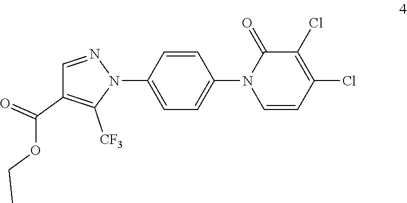 [SMILES: CCOC(=O)c1cnn(-c2ccc(-n3ccc(Cl)c(Cl)c3=O)cc2)c1C(F)(F)F]

Ethyl 1-(4-(3,4-dichloro-2-oxopyridin-1(2H)-yl)phenyl)-5-(trifluoromethyl)-1H-pyrazole-4-carboxylate (4). 48.5% yield; ¹H NMR (400 MHz, CDCl₃) δ 8.15 (s, 1H), 7.62-7.51 (m, 4H), 7.30 (d, J=7.5 Hz, 1H), 6.47 (d, J=7.5 Hz, 1H), 4.39 (q, J=7.1 Hz, 2H), 1.40 (t, J=7.1 Hz, 3H). HRMS (ESI): calcd for C₁₈H₁₃Cl₂F₃N₃O₃ 446.0281 [M+H]⁺, found 446.0269. Purity: 97.3% by HPLC (Rt=5.08 min).

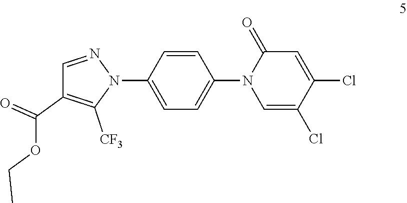 [SMILES: CCOC(=O)c1cnn(-c2ccc(-n3cc(Cl)c(Cl)cc3=O)cc2)c1C(F)(F)F]

Ethyl 1-(4-(4,5-dichloro-2-oxopyridin-1(2H)-yl)phenyl)-5-(trifluoromethyl)-1H-pyrazole-4-carboxylate (5). 21.3% yield; ¹H NMR (400 MHz, CDCl₃): δ 8.37 (s, 1H), 8.36 (s, 1H), 7.74-7.68 (m, 4H), 6.98 (s, 1H), 4.33 (q, J=7.2 Hz, 2H), 1.31 (t, J=7.2 Hz, 3H). HRMS (ESI): calcd for C₁₈H₁₃Cl₂F₃N₃O₃ 446.0281 [M+H]⁺, found 446.0268. Purity: 99.5% by HPLC (Rt=5.3 min).

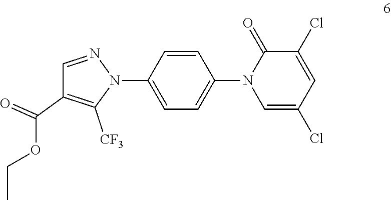 [SMILES: CCOC(=O)c1cnn(-c2ccc(-n3cc(Cl)cc(Cl)c3=O)cc2)c1C(F)(F)F]

Ethyl 1-(4-(3,5-dichloro-2-oxopyridin-1(2H)-yl)phenyl)-5-(trifluoromethyl)-1H-pyrazole-4-carboxylate (6). 40% yield; ¹H NMR (400 MHz, CDCl₃): δ 8.15 (s, 1H); 7.64 (d, J=2.4 Hz, 1H), 7.61-7.55 (m, 4H), 7.42 (d, J=2.4 Hz, 1H), 4.39 (q, J=7.2 Hz, 2H), 1.40 (t, J=7.2 Hz, 3H). HRMS (ESI): calcd for C₁₈H₁₃Cl₂F₃N₃O₃ 446.0281 [M+H]⁺, found 446.0300. Purity: 96.8% by HPLC (Rt=4.87 min).

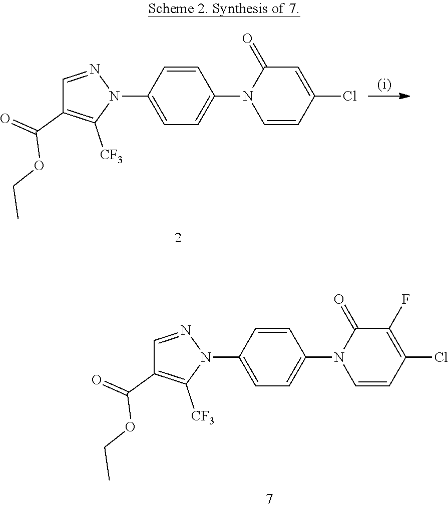 [SMILES: CCOC(=O)c1cnn(-c2ccc(-n3ccc(Cl)cc3=O)cc2)c1C(F)(F)F ; CCOC(=O)c1cnn(-c2ccc(-n3ccc(Cl)c(F)c3=O)cc2)c1C(F)(F)F]

Reagents and conditions: (i) Selectfluor, MeCN, 85° C., 2 h.

Fluorination of pyridone ring of 2 to form ethyl 1-(4-(4-chloro-3-fluoro-2-oxopyridin-1(2H)-yl)phenyl)-5-(trifluoromethyl)-1H-pyrazole-4-carboxylate (7). A mixture of compound 2 (2.05 g, 5 mmol) and Selectfluor [1-(chloromethyl)-4-fluoro-1,4-diazoniabicyclo[2.2.2]octanebis(tetrafluoroborate)] (3.55 g, 10 mmol) in MeCN (50 mL) was stirred at 85° C. for 2 h. The reaction was cooled to room temperature and concentrated under reduced pressure. The residue was dissolved in EtOAc (100 mL) and washed by water (3×50 mL). The extract was dried over MgSO₄ and concentrated under reduced pressure to give the crude material that was purified by silica gel flash column chromatography (CH₂Cl₂/acetone=19/1, v/v) to give the offwhite solid in 24.7% yield. ¹H NMR (400 MHz, CDCl₃) δ 8.15 (s, 1H), 7.65-7.51 (m, 4H), 7.18 (dd, J₁=7.6 Hz, J₂=1.9 Hz, 1H), 6.36 (dd, J₁=7.5 Hz, J₂=5.9 Hz, 1H), 4.39 (q, J=7.1 Hz, 2H), 1.40 (t, J=7.1 Hz, 3H).

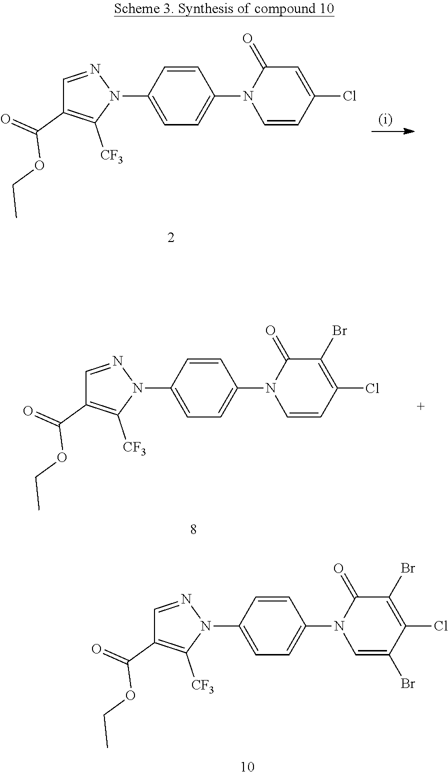 [SMILES: CCOC(=O)c1cnn(-c2ccc(-n3ccc(Cl)cc3=O)cc2)c1C(F)(F)F ; CCOC(=O)c1cnn(-c2ccc(-n3ccc(Cl)c(Br)c3=O)cc2)c1C(F)(F)F ; CCOC(=O)c1cnn(-c2ccc(-n3cc(Br)c(Cl)c(Br)c3=O)cc2)c1C(F)(F)F]

Reagents and conditions: (i) NBS, DMF, rt, overnight.

Bromination of pyridone ring of 2 to form monobromide and dibromide. Ethyl 1-(4-(4-chloro-2-oxopyridin-1(2H)-yl)phenyl)-5-(trifluoromethyl)-1H-pyrazole-4-carboxylate (2) (1.52 g, 3.69 mmol) and N-bromosuccinimide (0.49 g, 3.69 mmol) were dissolved in 50 mL of anhydrous DMF at room temperature under argon. The reaction mixture was stirred at room temperature overnight. The reaction was quenched by addition of 100 mL of water and extracted with ethyl acetate (3×50 mL). Organic layer was separated, washed with brine, dried over anhydrous MgSO₄, filtered and evaporated to an orange oil which was purified by column chromatography (silica gel, CH₂Cl₂/acetone=19/1 v/v) to give the desired compounds as white solids.

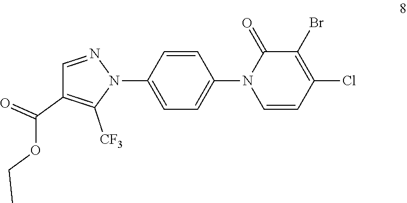 [SMILES: CCOC(=O)c1cnn(-c2ccc(-n3ccc(Cl)c(Br)c3=O)cc2)c1C(F)(F)F]

Ethyl 1-(4-(3-bromo-4-chloro-2-oxopyridin-1(2H)-yl)phenyl)-5-(trifluoromethyl)-1H-pyrazole-4-carboxylate (8). ¹H NMR (400 MHz, CDCl₃) δ 7.93 (s, 1H), 7.59 (d, J=8.7 Hz, 2H), 7.53 (d, J=8.8 Hz, 2H), 7.35 (d, J=7.4 Hz, 1H), 6.46 (d, J=7.4 Hz, 1H), 5.95 (s, 1H), 3.54-3.45 (m, 2H), 1.26 (t, J=7.3 Hz, 3H).

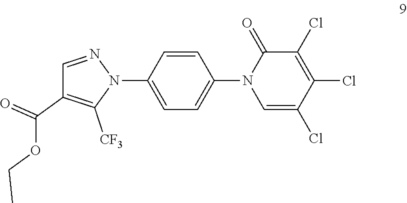 [SMILES: CCOC(=O)c1cnn(-c2ccc(-n3cc(Cl)c(Cl)c(Cl)c3=O)cc2)c1C(F)(F)F]

Ethyl 1-(4-(3,4,5-trichloro-2-oxopyridin-1(2H)-yl)phenyl)-5-(trifluoromethyl)-1H-pyrazole-4-carboxylate (9). 14.1% yield; ¹H NMR (400 MHz, CDCl₃) δ 8.44 (s, 1H), 8.36 (s, 1H), 7.77-7.71 (m, 4H), 4.33 (q, J=7.2 Hz, 2H), 1.31 (t, J=7.2 Hz, 3H). HRMS (ESI): calcd for C₁₈H₁₁Cl₃F₃N₃NaO₃ 501.9710 [M+Na]⁺, found 501.9704. Purity: 95.7% by HPLC (Rt=6.52 min).

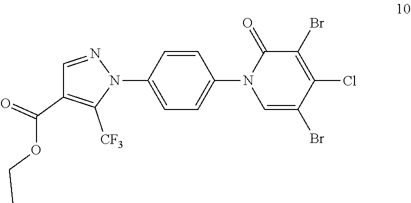 [SMILES: CCOC(=O)c1cnn(-c2ccc(-n3cc(Br)c(Cl)c(Br)c3=O)cc2)c1C(F)(F)F]

Ethyl 1-(4-(3,5-dibromo-4-chloro-2-oxopyridin-1(2H)-yl)phenyl)-5-(trifluoromethyl)-1H-pyrazole-4-carboxylate (10). ¹H NMR (400 MHz, CDCl₃) δ 8.15 (s, 1H), 7.70 (s, 1H), 7.60 (d, J=8.8 Hz, 2H), 7.55 (d, J=8.8 Hz, 2H), 4.39 (q, J=7.1 Hz, 2H), 1.40 (t, J=7.1 Hz, 3H).

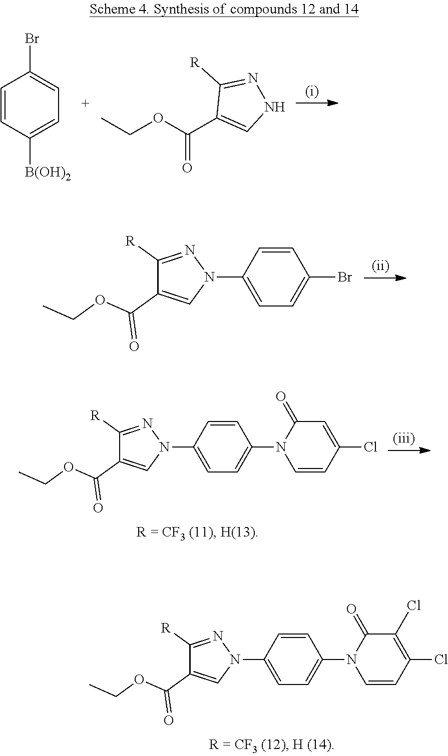

Reagents and conditions: (i) Cu(OAc)₂, Py, DMF, rt, 24 h; (ii) 4-chloropridinone, CuI, DMCDA, K₂CO₃, toluene, reflux, 12 h; (iii) NCS, DMF, 100° C., 8 h; (iv) 1 M KOH (aq.), EtOH, H₂O, rt, 8 h.

### General Procedure for Coupling Reaction to Form N-Substituted Pyrazole Derivatives

To a solution of pyrazoles (10 mmol) in DMF (50 mL) were added (4-bromophenyl)boronic acid (3.01 g, 15 mmol), cupric acetate (0.91 g, 5 mmol) and pyridine (791 mg, 10 mmol) and the resulting mixture was stirred at room temperature for 24 h. The precipitate was filtrated with Celite. The filtrate was diluted with water (100 mL) and extracted with ethyl acetate (3×30 mL). The organic layers were combined and washed with saturated aqueous sodium bicarbonate, dried over anhydrous magnesium sulfate, and concentrated in vacuo. The residue was purified by silica gel column chromatography to give the desired coupling products.

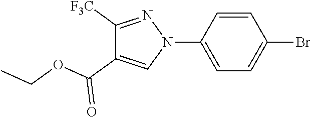 [SMILES: CCOC(=O)c1cn(-c2ccc(Br)cc2)nc1C(F)(F)F]

Ethyl 1-(4-bromophenyl)-3-(trifluoromethyl)-1H-pyrazole-4-carboxylate. 2.58 g, 71.8% yield; ¹H NMR (400 MHz, CDCl₃) δ 8.47 (d, J=0.9 Hz, 1H), 7.68-7.58 (m, 4H), 4.37 (q, J=7.1 Hz, 2H), 1.39 (t, J=7.1 Hz, 3H).

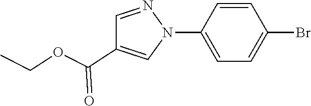 [SMILES: CCOC(=O)c1cnn(-c2ccc(Br)cc2)c1]

Ethyl 1-(4-bromophenyl)-1H-pyrazole-4-carboxylate. 1.86 g, 63.4% yield; ¹H NMR (400 MHz, CDCl₃) δ 8.39 (s, 1H), 8.10 (s, 1H), 7.61 (s, 4H), 4.34 (q, J=7.1 Hz, 2H), 1.38 (t, J=7.1 Hz, 3H).

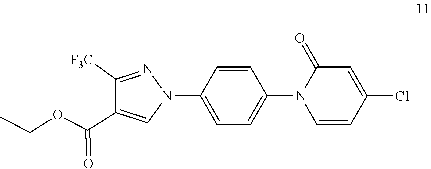 [SMILES: CCOC(=O)c1cn(-c2ccc(-n3ccc(Cl)cc3=O)cc2)nc1C(F)(F)F]

Ethyl 1-(4-(4-chloro-2-oxopyridin-1(2H)-yl)phenyl)-3-(trifluoromethyl)-1H-pyrazole-4-carboxylate (11). 53.1% yield; ¹H NMR (400 MHz, CDCl₃) δ 8.52 (d, J=0.9 Hz, 1H), 7.88 (d, J=8.9 Hz, 2H), 7.55 (d, J=8.9 Hz, 2H), 7.31 (d, J=7.1 Hz, 1H), 6.74 (d, J=2.0 Hz, 1H), 6.35 (dd, J₁=7.4 Hz, J₂=2.2 Hz, 1H), 4.38 (q, J=7.1 Hz, 2H), 1.40 (t, J=7.1 Hz, 3H).

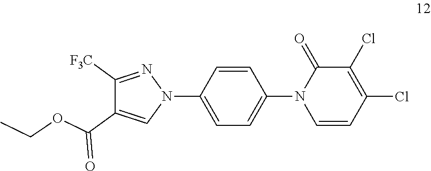 [SMILES: CCOC(=O)c1cn(-c2ccc(-n3ccc(Cl)c(Cl)c3=O)cc2)nc1C(F)(F)F]

Ethyl 1-(4-(3,4-dichloro-2-oxopyridin-1(2H)-yl)phenyl)-3-(trifluoromethyl)-1H-pyrazole-4-carboxylate (12). 38.6% yield; ¹H NMR (400 MHz, CDCl₃) δ 8.54 (s, 1H), 7.89 (d, J=8.8 Hz, 2H), 7.55 (d, J=8.8 Hz, 2H), 7.28 (d, J=7.5 Hz, 1H), 6.46 (d, J=7.5 Hz, 1H), 4.38 (q, J=7.1 Hz, 2H), 1.40 (t, J=7.1 Hz, 3H).

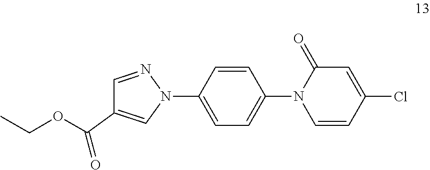 [SMILES: CCOC(=O)c1cnn(-c2ccc(-n3ccc(Cl)cc3=O)cc2)c1]

Ethyl 1-(4-(4-chloro-2-oxopyridin-1(2H)-yl)phenyl)-1H-pyrazole-4-carboxylate (13). 48.9% yield; ¹H NMR (400 MHz, CDCl₃) δ 8.44 (s, 1H), 8.13 (s, 1H), 7.86 (d, J=8.7 Hz, 2H), 7.51 (d, J=8.8 Hz, 2H), 7.32 (d, J=7.4 Hz, 1H), 6.74 (d, J=2.2 Hz, 1H), 6.33 (dd, J=7.4 Hz, J₂=2.2 Hz, 1H), 4.36 (q, J=7.1 Hz, 2H), 1.39 (t, J=7.1 Hz, 3H).

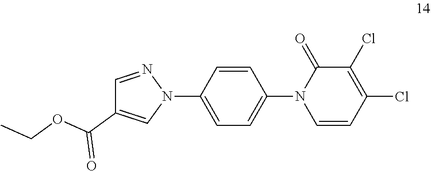 [SMILES: CCOC(=O)c1cnn(-c2ccc(-n3ccc(Cl)c(Cl)c3=O)cc2)c1]

Ethyl 1-(4-(3,4-dichloro-2-oxopyridin-1(2H)-yl)phenyl)-1H-pyrazole-4-carboxylate (14). 44.3% yield; ¹H NMR (400 MHz, DMSO-d₆) δ 8.28 (s, 1H), 7.88 (d, J=7.5 Hz, 1H), 7.79 (d, J=8.5 Hz, 2H), 7.70 (d, J=8.7 Hz, 2H), 6.69 (d, J=7.4 Hz, 1H), 4.30 (q, J=7.0 Hz, 2H), 1.30 (t, J=7.1 Hz, 3H).

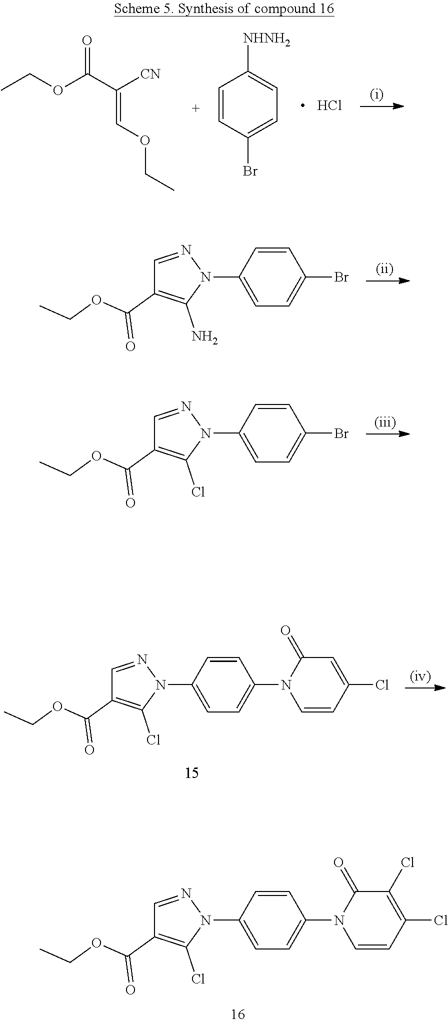

Reagents and conditions: (i) EtOH, 60° C., 6 h; (ii) conc. HCl, NaNO₂, CuCl₂, 60° C., overnight; (iii) 4-chloropridinone, CuI, DMCDA, K₂CO₃, toluene, reflux, 12 h; (iv) NCS, DMF, 100° C., 8 h.

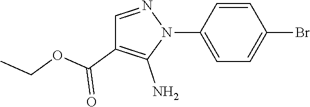 [SMILES: CCOC(=O)c1cnn(-c2ccc(Br)cc2)c1N]

Ethyl 5-amino-1-(4-bromophenyl)-1H-pyrazole-4-carboxylate. 87.4% yield; ¹H NMR (400 MHz, DMSO-d₆) δ 7.92-7.65 (m, 3H), 7.50 (d, J=8.7 Hz, 2H), 6.41 (s, 2H), 4.20 (q, J=7.1 Hz, 2H), 1.25 (t, J=7.1 Hz, 3H).

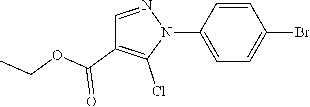 [SMILES: CCOC(=O)c1cnn(-c2ccc(Br)cc2)c1Cl]

Ethyl 1-(4-bromophenyl)-5-chloro-1H-pyrazole-4-carboxylate. Ethyl 5-amino-1-(4-bromophenyl)-1H-pyrazole-4-carboxylate (3.1 g, 10 mmol) was dissolved in a concentrated hydrochloric acid (50 mL) and stirred at 0° C. Sodium nitrite (828 mg, 12 mmol) was subsequently added and stirred at 0° C. for 1 h. After addition of copper (II) chloride (2.02 g, 15 mmol) the temperature was allowed to rise to 60° C. and the reaction was stirred overnight. The resulting solution was poured into ice water (200 mL) and then extracted with ethyl acetate (3×50 mL). The obtained organic layers were washed with saturated sodium bicarbonate solution, dried over anhydrous magnesium sulfate, and filtered. The filtrate was concentrated under reduced pressure, and the obtained residue was purified by silica gel column chromatography to afford 2.04 g (62.9% yield) of the target product. ¹H NMR (400 MHz, CDCl₃) δ 8.11 (s, 1H), 7.65 (d, J=8.6 Hz, 2H), 7.44 (d, J=8.6 Hz, 2H), 4.36 (q, J=7.1 Hz, 2H), 1.39 (t, J=7.1 Hz, 3H).

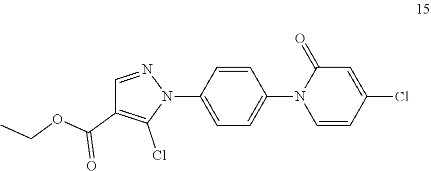 [SMILES: CCOC(=O)c1cnn(-c2ccc(-n3ccc(Cl)cc3=O)cc2)c1Cl]

Ethyl 5-chloro-1-(4-(4-chloro-2-oxopyridin-1(2H)-yl)phenyl)-1H-pyrazole-4-carboxylate (15). 38.6% yield; ¹H NMR (400 MHz, CDCl₃) δ 8.14 (s, 1H), 7.72 (d, J=8.7 Hz, 2H), 7.54 (d, J=8.7 Hz, 2H), 7.32 (d, J=7.4 Hz, 1H), 6.74 (d, J=2.1 Hz, 1H), 6.34 (dd, J₁=7.4, J₂=2.1 Hz, 1H), 4.37 (q, J=7.1 Hz, 2H), 1.40 (t, J=7.1 Hz, 3H).

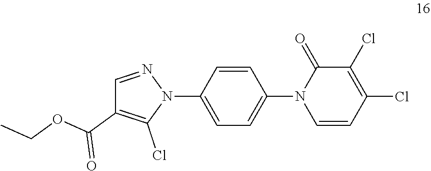 [SMILES: CCOC(=O)c1cnn(-c2ccc(-n3ccc(Cl)c(Cl)c3=O)cc2)c1Cl]

Ethyl 5-chloro-1-(4-(3,4-dichloro-2-oxopyridin-1(2H)-yl)phenyl)-1H-pyrazole-4-carboxylate (16). 31.3% yield; ¹H NMR (400 MHz, DMSO-d₆) δ 8.28 (s, 1H), 7.88 (d, J=7.5 Hz, 1H), 7.79 (d, J=8.5 Hz, 2H), 7.70 (d, J=8.7 Hz, 2H), 6.69 (d, J=7.4 Hz, 1H), 4.30 (q, J=7.0 Hz, 2H), 1.30 (t, J=7.1 Hz, 3H).

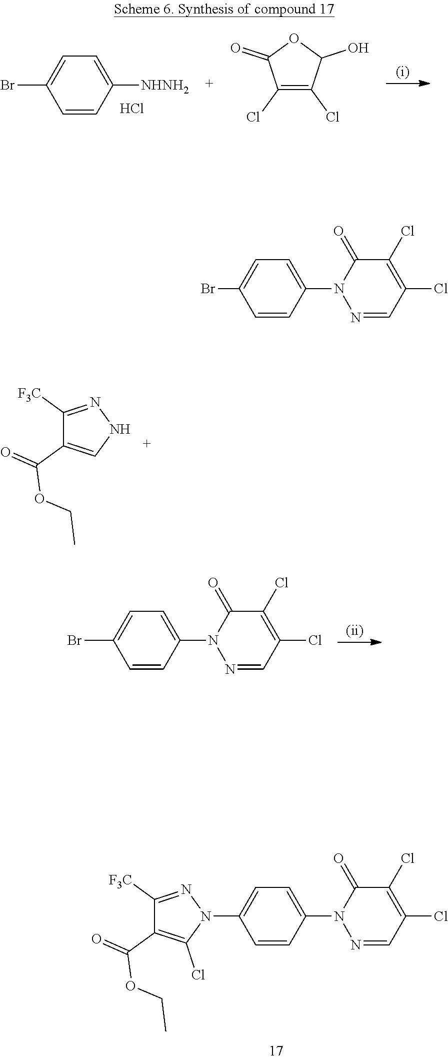

Reagents and conditions: (i) H₂O, 90° C., 6 h; (ii) CuI, DMCDA, K₂CO₃, toluene, reflux, 12 h.

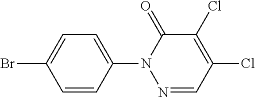 [SMILES: O=c1c(Cl)c(Cl)cnn1-c1ccc(Br)cc1]

2-(4-bromophenyl)-4,5-dichloropyridazin-3(2H)-one. After 4-bromophenyl)hydrazine hydrochloride (1.12 g, 5 mmol) dissolved in water (100 mL) at 60° C., mucochloric acid (0.84 g, 5 mmol) was added portion-wise and the reaction was heated up to 90° C. for 6 h. After cooling down, the orange precipitate was filtered, dried in vacuo, and used directly in next step without further purification (1.07 g, 67.2%). ¹H NMR (400 MHz, CDCl₃) δ 7.93 (s, 1H), 7.61 (d, J=8.8 Hz, 2H), 7.50 (d, J=8.8 Hz, 2H).

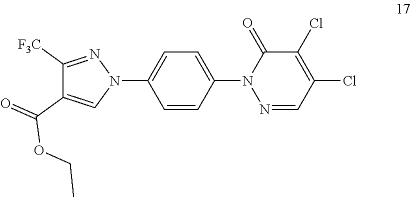 [SMILES: CCOC(=O)c1cn(-c2ccc(-n3ncc(Cl)c(Cl)c3=O)cc2)nc1C(F)(F)F]

Ethyl 1-(4-(4,5-dichloro-6-oxopyridazin-1(6H)-yl)phenyl)-3-(trifluoromethyl)-1H-pyrazole-4-carboxylate (17). 28.5% yield; ¹H NMR (400 MHz, CDCl₃) δ 8.54 (s, 1H), 7.97 (s, 1H), 7.89-7.79 (m, 4H), 4.38 (q, J=7.1 Hz, 2H), 1.39 (t, J=7.1 Hz, 3H).

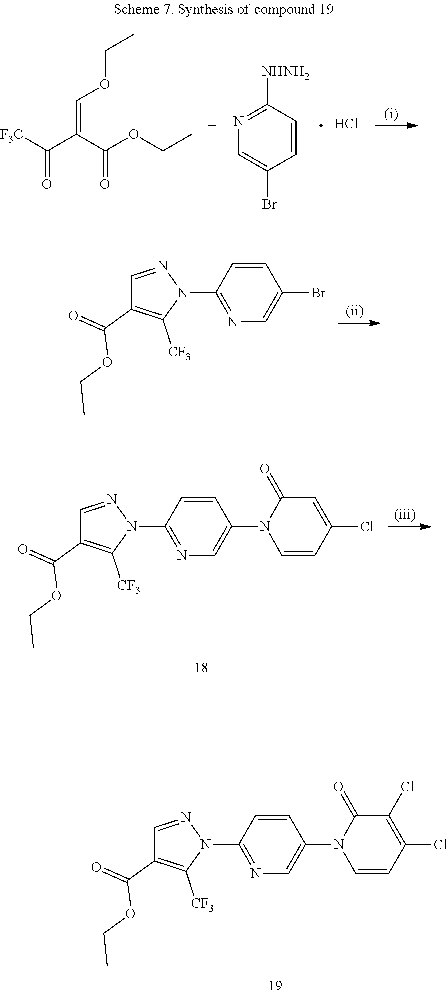 [SMILES: CCO/C=C(\C(=O)OCC)C(=O)C(F)(F)F ; NNc1ccc(Br)cn1 ; Cl ; CCOC(=O)c1cnn(-c2ccc(Br)cn2)c1C(F)(F)F ; CCOC(=O)c1cnn(-c2ccc(-n3ccc(Cl)cc3=O)cn2)c1C(F)(F)F ; CCOC(=O)c1cnn(-c2ccc(-n3ccc(Cl)c(Cl)c3=O)cn2)c1C(F)(F)F]

Reagents and conditions: (i) EtOH, 60° C., 6 h; (ii) 4-chloropridinone, CuI, DMCDA, K₂CO₃, toluene, reflux, 12 h; (iii) NCS, DMF, 100° C., 8 h.

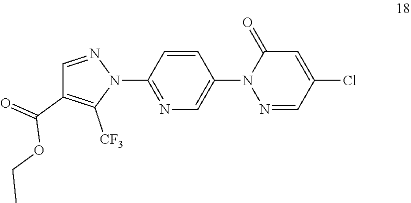 [SMILES: CCOC(=O)c1cnn(-c2ccc(-n3ncc(Cl)cc3=O)cn2)c1C(F)(F)F]

Ethyl 1-(4-chloro-2-oxo-2H-[1,3′-bipyridin]-6′-yl)-5-(trifluoromethyl)-1H-pyrazole-4-carboxylate (18). 59.3% yield; ¹H NMR (400 MHz, CDCl₃) δ 8.55 (d, J=2.5 Hz, 1H), 8.14 (s, 1H), 8.08 (dd, J₁=8.6 Hz, J₂=2.6 Hz, 1H), 7.83 (d, J=8.6 Hz, 1H), 7.35 (d, J=7.4 Hz, 1H), 6.76 (d, J=2.1 Hz, 1H), 6.40 (dd, J₁=7.4, J₂=2.1 Hz, 1H), 4.39 (q, J=7.1 Hz, 2H), 1.40 (t, J=7.1 Hz, 3H).

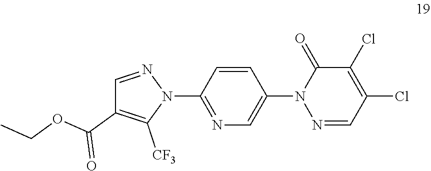 [SMILES: CCOC(=O)c1cnn(-c2ccc(-n3ncc(Cl)c(Cl)c3=O)cn2)c1C(F)(F)F]

Ethyl 1-(3,4-dichloro-2-oxo-2H-[1,3′-bipyridin]-6′-yl)-5-(trifluoromethyl)-1H-pyrazole-4-carboxylate (19). 38.8% yield; ¹H NMR (400 MHz, DMSO-d⁶) δ 8.75 (d, J=2.4 Hz, 1H), 8.42 (s, 1H), 8.33 (dd, J₁=8.6 Hz, J₂=2.5 Hz, 1H), 8.03 (d, J=8.5 Hz, 1H), 7.98 (d, J=7.5 Hz, 1H), 6.77 (d, J=7.5 Hz, 1H), 4.34 (q, J=7.1 Hz, 2H), 1.32 (t, J=7.1 Hz, 3H).

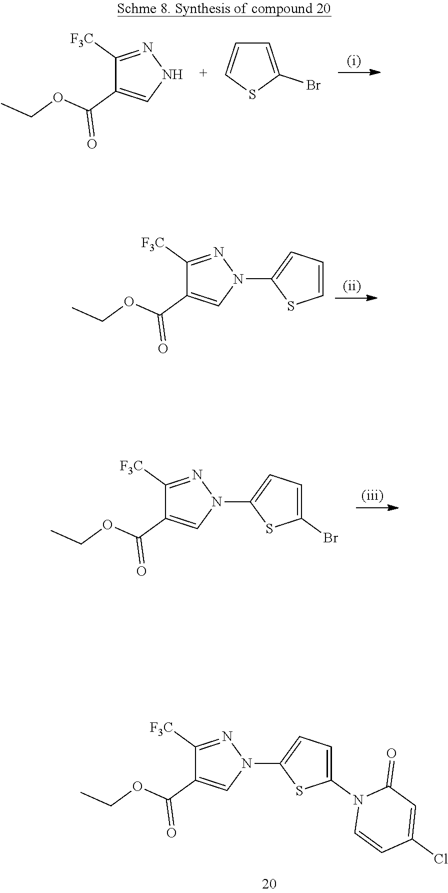 [SMILES: CCOC(=O)c1c[nH]nc1C(F)(F)F ; Brc1cccs1 ; CCOC(=O)c1cn(-c2cccs2)nc1C(F)(F)F ; CCOC(=O)c1cn(-c2ccc(Br)s2)nc1C(F)(F)F ; CCOC(=O)c1cn(-c2ccc(-n3ccc(Cl)cc3=O)s2)nc1C(F)(F)F]

Reagents and conditions: (i) Cu₂O, K₂CO₃, DMF, 120° C., 18 h; (ii) NBS, CH₂Cl₂, rt, overnight; (iii) 4-chloropridinone, CuI, DMCDA, K₂CO₃, toluene, reflux, 12 h.

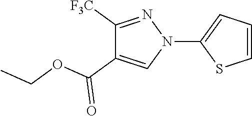 [SMILES: CCOC(=O)c1cn(-c2cccs2)nc1C(F)(F)F]

Ethyl 1-(thiophen-2-yl)-3-(trifluoromethyl)-1H-pyrazole-4-carboxylate. Dry DMF (30 mL) was added to a mixture of ethyl 3-(trifluoromethyl)-1H-pyrazole-4-carboxylate (2.08 g, 10 mmol), 2-bromothiophene (2.12 g, 1.3 mmol), Cu₂O (286 mg, 2 mmol), and K₂CO₃ (4.89 g, 15 mmol) under argon. The heterogeneous mixture was stirred at 120° C. for 18 h. after cooling down, the reaction was diluted with ethyl acetate (50 mL) and was washed with water (200 mL×3). The organic layer was dried over Na2SO4 and concentrated in vacuo. The residue was purified by flash chromatography to obtain the product as a white solid (1.18 g, 33.1%). ¹H NMR (400 MHz, CDCl₃) δ 8.35 (s, 1H), 7.24-7.17 (m, 2H), 7.01 (dd, J₁=5.4 Hz, J₂=3.8 Hz, 1H), 4.37 (d, J=7.1 Hz, 2H), 1.38 (t, J=7.1 Hz, 3H).

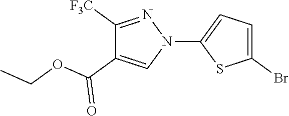 [SMILES: CCOC(=O)c1cn(-c2ccc(Br)s2)nc1C(F)(F)F]

Ethyl 1-(5-bromothiophen-2-yl)-3-(trifluoromethyl)-1H-pyrazole-4-carboxylate. To a solution of (1.07 g, 3 mmol) in CH2Cl2 (50 mL) at 0° C. was added NBS (534 mg, 3 mmol). The reaction was allowed to warm up to room temperature and stirred overnight. Water (100 mL) was added to the solution and the organic layer was separated, dried over Na2SO₄, and concentrated under reduced pressure. The residue was purified by flash chromatography to give the product as a white solid (1.19 g, 91.4%). ¹H NMR (400 MHz, CDCl₃) δ 8.30 (d, J=0.9 Hz, 1H), 7.00 (d, J=4.1 Hz, 1H), 6.96 (d, J=4.1 Hz, 1H), 4.36 (q, J=7.1 Hz, 2H), 1.38 (t, J=7.1 Hz, 3H).

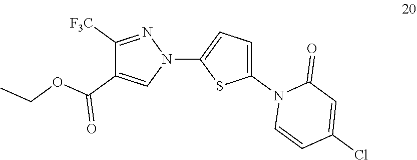 [SMILES: CCOC(=O)c1cn(-c2ccc(-n3ccc(Cl)cc3=O)s2)nc1C(F)(F)F]

Ethyl 1-(5-(4-chloro-2-oxopyridin-1(2H)-yl)thiophen-2-yl)-3-(trifluoromethyl)-1H-pyrazole-4-carboxylate (20). 16.1% yield; ¹H NMR (400 MHz, CDCl₃) δ 8.37 (d, J=0.8 Hz, 1H), 7.66 (d, J=7.6 Hz, 1H), 7.16 (d, J=4.4 Hz, 1H), 7.01 (d, J=4.4 Hz, 1H), 6.80 (d, J=2.1 Hz, 1H), 6.42 (dd, J₁=7.6 Hz, J₂=2.3 Hz, 1H), 4.37 (q, J=7.1 Hz, 2H), 1.38 (t, J=7.1 Hz, 3H).

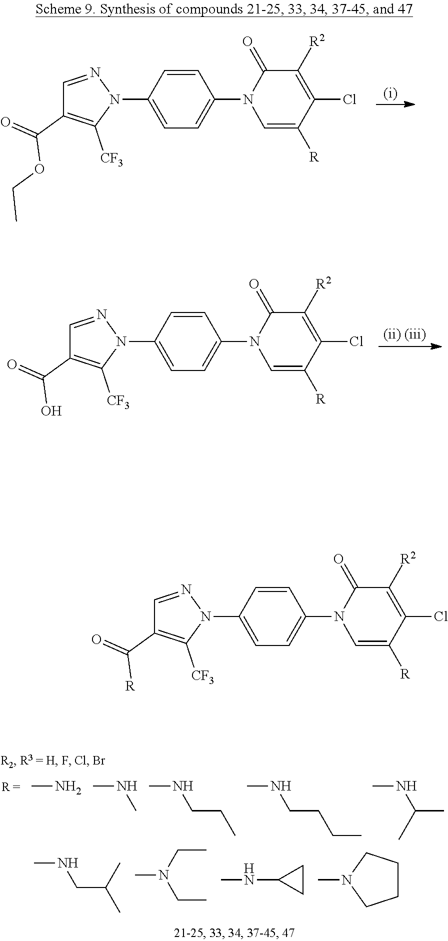

Reagents and conditions: (i) 1 M KOH (aq.), EtOH, H₂O, rt, 8 h; (ii) SOCl₂, DCM, reflux, 4 h; (iii) amines, TEA, THF, r.t., 12 h.

### General Procedure for Hydrolysis of the Esters to Carboxylic Acids.

The ester compound (1.17 mmol) was dissolved in 10 mL of ethanol at room temperature. To this solution was added 2.33 mL of 1M KOH solution at room temperature. The resulted mixture was stirred at room temperature for 8 hours. The reaction solution was neutralized to PH=6 by adding saturated NH₄Cl solution and extracted with Ethyl acetate (3×50 mL). The organic layers were combined, dried over anhydrous MgSO₄, filtered and concentrated to dryness under reduced pressure. The crude solids were used directly in next step without further purification.

### General Procedure for Synthesis of Amides.

Thionyl chloride (1M in CH₂Cl₂, 1.4 mL) was added dropwise to a solution of carboxylic acid (1.15 mmol) in CH₂Cl₂ (20 mL)/DMF (0.1 mL). The reaction mixture was stirred at reflux for 4 h. The volatile was removed under reduced pressure and the residue with ethylamine (0.69 mL of 2M THF solution, 1.38 mmol) were mixed together in 40 mL of anhydrous THF at 0° C. in an ice bath. Triethylamine (0.35 g, 3.45 mmol) was added dropwise with stirring and the resulting mixture was slowly warmed to room temperature and stirred overnight. The solvent was evaporated, and the residue purified by column chromatography using a mixture of CH₂Cl₂/MeOH (19/1, v/v) to afford the desired compounds as white solids.

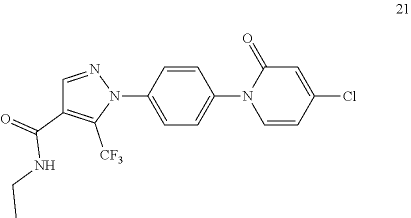 [SMILES: CCNC(=O)c1cnn(-c2ccc(-n3ccc(Cl)cc3=O)cc2)c1C(F)(F)F]

1-(4-(4-chloro-2-oxopyridin-1(2H)-yl)phenyl)-N-ethyl-5-(trifluoromethyl)-1H-pyrazole-4-carboxamide (21). 70.3% yield for two steps; ¹H NMR (400 MHz, DMSO-d₆) δ 8.59 (t, J=5.5 Hz, 1H), 8.16 (s, 1H), 7.87 (d, J=7.4 Hz, 1H), 7.66 (s, 4H), 6.72 (d, J=2.3 Hz, 1H), 6.50 (dd, J₁=7.4 Hz, J₂=2.3 Hz, 1H), 3.31-3.20 (m, 2H), 1.11 (t, J=7.2 Hz, 3H). HRMS (ESI): calcd for C₁₈H₁₅ClF₃N₄O₂ 411.0830 [M+H]⁺, found 411.0836. Purity: 97.5% by HPLC (Rt=3.06 min).

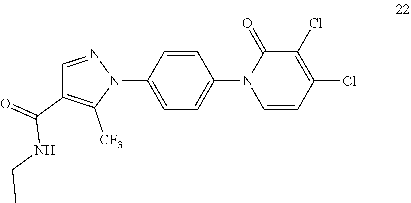 [SMILES: CCNC(=O)c1cnn(-c2ccc(-n3ccc(Cl)c(Cl)c3=O)cc2)c1C(F)(F)F]

1-(4-(3,4-dichloro-2-oxopyridin-1(2H)-yl)phenyl)-5-(trifluoromethyl)-1H-pyrazole-4-carboxamide (22). 63.0% yield for two steps; ¹H NMR (400 MHz, CDCl₃): δ 8.59 (t, J=5.2 Hz, 1H), 8.17 (s, 1H), 7.89 (d, J=7.6 Hz, 1H), 7.70 (s, 4H), 6.70 (d, J=7.2 Hz, 1H), 3.29-3.23 (m, 2H), 1.12 (t, J=7.2 Hz, 3H). HRMS (ESI): calcd for C₁₈H₁₂Cl₂F₃N₃O₃ 445.0440 [M+H]⁺, found 445.0444. Purity: 99.5% by HPLC (Rt=3.45 min).

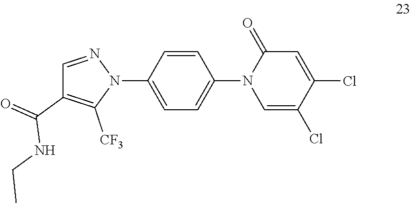 [SMILES: CCNC(=O)c1cnn(-c2ccc(-n3cc(Cl)c(Cl)cc3=O)cc2)c1C(F)(F)F]

1-(4-(4,5-dichloro-2-oxopyridin-1(2H)-yl)phenyl)-5-(trifluoromethyl)-1H-pyrazole-4-carboxamide (23). 67.6% yield for two steps; ¹H NMR (400 MHz, DMSO-d₆) δ 8.58 (t, J=5.5 Hz, 1H), 8.35 (s, 1H), 8.15 (s, 1H), 7.77-7.58 (m, 4H), 6.97 (s, 1H), 3.30-3.19 (m, 2H), 1.10 (t, J=7.2 Hz, 4H). HRMS (ESI): calcd for C₁₈H₁₄Cl₂F₃N₄O₂ 445.0440 [M+H]⁺, found 445.0402. Purity: 97.5% by HPLC (Rt=3.35 min).

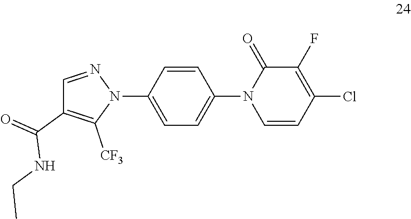 [SMILES: CCNC(=O)c1cnn(-c2ccc(-n3ccc(Cl)c(F)c3=O)cc2)c1C(F)(F)F]

1-(4-(4-chloro-3-fluoro-2-oxopyridin-1(2H)-yl)phenyl)-5-(trifluoromethyl)-1H-pyrazole-4-carboxamide (24). 71.6% yield for two steps; ¹H NMR (400 MHz, DMSO-d₆) δ 8.58 (t, J=5.5 Hz, 1H), 8.16 (s, 1H), 7.74 (dd, J₁=7.6 Hz, J₂=1.9 Hz, 1H), 7.69 (s, 3H), 6.59 (dd, J₁=7.5 Hz, J₂=6.3 Hz, 1H), 3.30-3.20 (m, 2H), 1.10 (t, J=7.2 Hz, 3H). ¹⁹F NMR (376 MHz, DMSO-d₆) δ −54.38, −134.40. HRMS (ESI): calcd for C₁₈H₁₂Cl₂F₃N₃O₃ 429.0736 [M+H]⁺, found 429.0738.

Purity: 96.2% by HPLC (Rt=3.12 min).

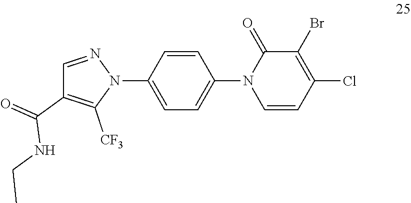 [SMILES: CCNC(=O)c1cnn(-c2ccc(-n3ccc(Cl)c(Br)c3=O)cc2)c1C(F)(F)F]

1-(4-(3-bromo-4-chloro-2-oxopyridin-1(2H)-yl)phenyl)-N-ethyl-5-(trifluoromethyl)-1H-pyrazole-4-carboxamide (25). ¹H NMR (400 MHz, CDCl₃) δ 7.93 (s, 1H), 7.59 (d, J=8.7 Hz, 2H), 7.53 (d, J=8.8 Hz, 2H), 7.35 (d, J=7.4 Hz, 1H), 6.46 (d, J=7.4 Hz, 1H), 5.95 (s, 1H), 3.54-3.45 (m, 2H), 1.26 (t, J=7.3 Hz, 4H).

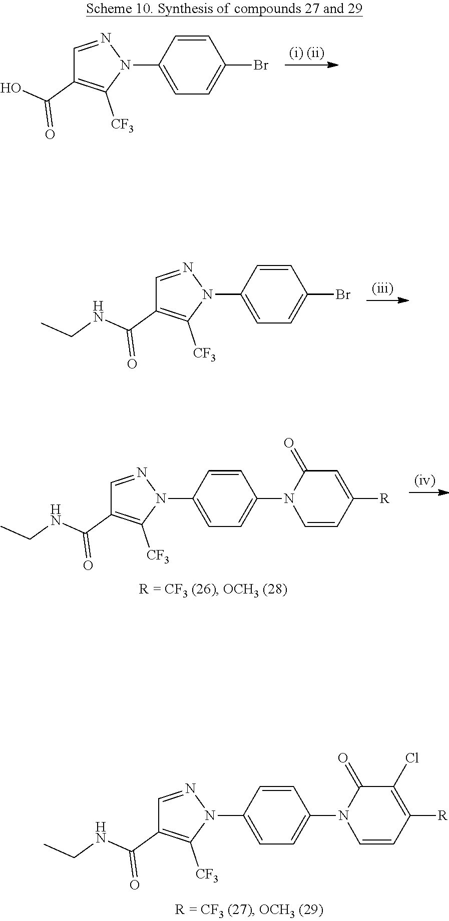

Reagents and conditions: (i) SOCl₂, CH₂Cl₂, reflux, 4 h; (ii) ethylamine, TEA, THF, r.t., 12 h; (iii) 4-methoxypyridinone or 4-(trifluoromethyl)pyridinone, CuI, DMCDA, K₂CO₃, toluene, reflux, 12 h; (iv) NCS, DMF, 100° C., 8 h.

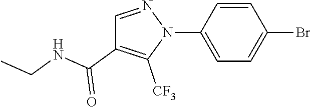 [SMILES: CCNC(=O)c1cnn(-c2ccc(Br)cc2)c1C(F)(F)F]

1-(4-bromophenyl)-N-ethyl-5-(trifluoromethyl)-1H-pyrazole-4-carboxamide. 92.2% yield; ¹H NMR (400 MHz, CDCl₃) δ 7.90 (s, 1H), 7.63 (d, J=8.6 Hz, 1H), 7.50 (dd, J₁=5.5 Hz, J₂=3.7 Hz, 1H), 7.47-7.38 (m, 1H), 7.31 (d, J=8.5 Hz, 1H), 6.15 (s, 1H), 3.51-3.41 (m, 2H), 1.24 (t, J=7.3 Hz, 3H).

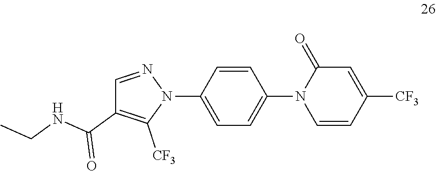 [SMILES: CCNC(=O)c1cnn(-c2ccc(-n3ccc(C(F)(F)F)cc3=O)cc2)c1C(F)(F)F]

N-ethyl-1-(4-(2-oxo-4-(trifluoromethyl)pyridin-1(2H)-yl)phenyl)-5-(trifluoromethyl)-1H-pyrazole-4-carboxamide (26). 32.6% yield; ¹H NMR (400 MHz, DMSO-d₆) δ 8.58 (t, J=5.5 Hz, 1H), 8.16 (s, 1H), 8.03 (d, J=7.2 Hz, 1H), 7.75-7.65 (m, 4H), 6.96 (s, 1H), 6.59 (dd, J₁=7.2 Hz, J₂=1.9 Hz, 1H), 3.29-3.19 (m, 2H), 1.11 (t, J=7.2 Hz, 3H).

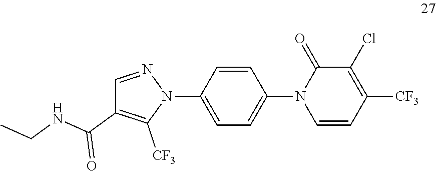 [SMILES: CCNC(=O)c1cnn(-c2ccc(-n3ccc(C(F)(F)F)c(Cl)c3=O)cc2)c1C(F)(F)F]

1-(4-(3-chloro-2-oxo-4-(trifluoromethyl)pyridin-1(2H)-yl)phenyl)-N-ethyl-5-(trifluoromethyl)-1H-pyrazole-4-carboxamide (27). 68.1% yield; ¹H NMR (400 MHz, DMSO-d₆) δ 8.59 (t, J=5.3 Hz, 1H), 8.16 (s, 1H), 8.03 (d, J=7.3 Hz, 1H), 7.76-7.66 (m, 4H), 6.69 (d, J=7.3 Hz, 1H), 3.22 (q, J=7.1 Hz, 2H), 1.10 (t, J=7.2 Hz, 3H).

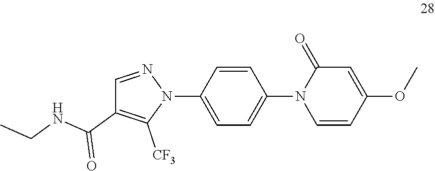 [SMILES: CCNC(=O)c1cnn(-c2ccc(-n3ccc(OC)cc3=O)cc2)c1C(F)(F)F]

N-ethyl-1-(4-(4-methoxy-2-oxopyridin-1(2H)-yl)phenyl)-5-(trifluoromethyl)-1H-pyrazole-4-carboxamide (28). 57.0% yield; ¹H NMR (400 MHz, DMSO-d₆) δ 8.58 (t, J=5.4 Hz, 1H), 8.14 (s, 1H), 7.70-7.56 (m, 6H), 6.08 (dd, J=7.7 Hz, J₂=2.7 Hz, 1H), 5.92 (d, J=2.7 Hz, 1H), 3.25 (q, J=7.1 Hz, 2H), 1.10 (t, J=7.2 Hz, 3H).

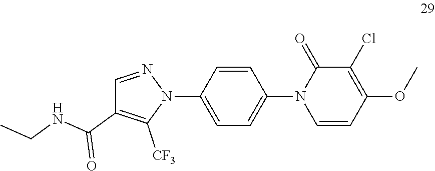 [SMILES: CCNC(=O)c1cnn(-c2ccc(-n3ccc(OC)c(Cl)c3=O)cc2)c1C(F)(F)F]

1-(4-(3-chloro-4-methoxy-2-oxopyridin-1(2H)-yl)phenyl)-N-ethyl-5-(trifluoromethyl)-1H-pyrazole-4-carboxamide (29). 49.5% yield; ¹H NMR (400 MHz, DMSO-d₆) δ 8.58 (t, J=5.5 Hz, 1H), 8.15 (s, 1H), 7.90 (d, J=7.8 Hz, 1H), 7.70-7.59 (m, 4H), 6.59 (d, J=7.9 Hz, 1H), 3.99 (s, 3H), 3.29-3.20 (m, 2H), 1.11 (t, J=7.2 Hz, 3H).

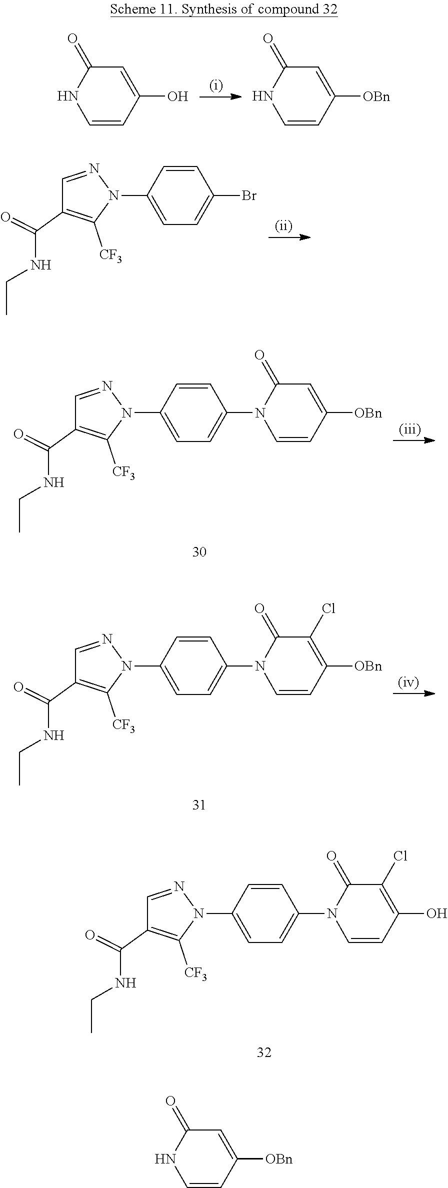

4-(benzyloxy)pyridin-2(1H)-one. To a solution of 4-hydroxypyridin-2(1H)-one (1.11 g, 10 mmol) in DMF (10 mL) were added Benzyl bromide (1.71 g, 10 mmol) and potassium carbonate (2.76 g, 20 mmol). The reaction mixture was stirred overnight at 60° C. After filtration and evaporation of the solvent, the residue was purified by flash chromatography to afford desired product as white solid (864 mg, 43.4% yield). ¹H NMR (400 MHz, CDCl₃) δ 7.47-7.32 (m, 5H), 7.23 (d, J=7.3 Hz, 1H), 6.05 (dd, J=7.3 Hz, J₂=2.4 Hz, 1H), 5.98 (d, J=2.4 Hz, 1H), 5.02 (s, 2H).

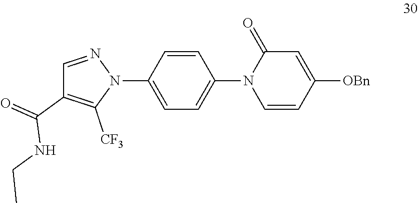 [SMILES: CCNC(=O)c1cnn(-c2ccc(-n3ccc(OCc4ccccc4)cc3=O)cc2)c1C(F)(F)F]

1-(4-(4-(benzyloxy)-2-oxopyridin-1(2H)-yl)phenyl)-N-ethyl-5-(trifluoromethyl)-1H-pyrazole-4-carboxamide (30). 50.5% yield; ¹H NMR (400 MHz, DMSO-d₆) δ 8.57 (t, J=5.5 Hz, 1H), 8.14 (s, 1H), 7.68 (d, J=7.7 Hz, 1H), 7.65-7.56 (m, 4H), 7.49-7.34 (m, 5H), 6.14 (dd, J=7.7 Hz, J₂=2.6 Hz, 1H), 6.03 (d, J=2.6 Hz, 1H), 5.15 (s, 2H), 3.29-3.19 (m, 2H), 1.10 (t, J=7.2 Hz, 3H).

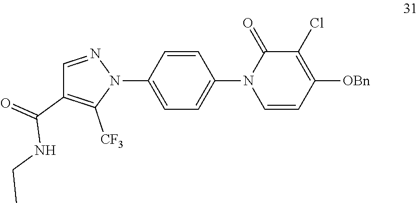 [SMILES: CCNC(=O)c1cnn(-c2ccc(-n3ccc(OCc4ccccc4)c(Cl)c3=O)cc2)c1C(F)(F)F]

1-(4-(4-(benzyloxy)-3-chloro-2-oxopyridin-1(2H)-yl)phenyl)-N-ethyl-5-(trifluoromethyl)-1H-pyrazole-4-carboxamide (31). 74.4% yield; ¹H NMR (400 MHz, DMSO-d₆) δ 8.58 (s, 1H), 8.15 (s, 1H), 7.88 (d, J=7.8 Hz, 1H), 7.64 (s, 4H), 7.51-7.33 (m, 5H), 6.65 (d, J=7.9 Hz, 1H), 5.40 (s, 2H), 3.25 (dt, J₁=14.0 Hz, J₂=7.1 Hz, 2H), 1.10 (t, J=7.2 Hz, 3H).

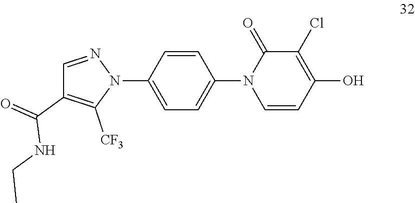 [SMILES: CCNC(=O)c1cnn(-c2ccc(-n3ccc(O)c(Cl)c3=O)cc2)c1C(F)(F)F]

1-(4-(3-chloro-4-hydroxy-2-oxopyridin-1(2H)-yl)phenyl)-N-ethyl-5-(trifluoromethyl)-1H-pyrazole-4-carboxamide (32). 1-(4-(4-(benzyloxy)-3-chloro-2-oxopyridin-1(2H)-yl)phenyl)-N-ethyl-5-(trifluoromethyl)-1H-pyrazole-4-carboxamide (51.7 mg, 0.1 mmol) was dissolved in methanol (10 mL) and Pd/C (10 wt. %, 5 mg) was successively added. The mixture was stirred at room temperature under hydrogen atmosphere for 2 h. After filtration, the volatiles were removed in vacuo and the residue was purified by flash chromatography to afford product as white solid (35.4 mg, 83.1% yield). ¹H NMR (400 MHz, DMSO-d₆) δ 8.62 (t, J=5.5 Hz, 1H), 8.19 (s, 1H), 7.73-7.60 (m, 5H), 6.25 (d, J=7.6 Hz, 1H), 3.29-3.22 (m, 2H), 1.15 (t, J=7.2 Hz, 3H).

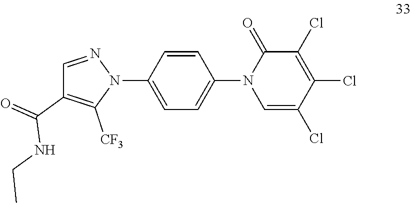 [SMILES: CCNC(=O)c1cnn(-c2ccc(-n3cc(Cl)c(Cl)c(Cl)c3=O)cc2)c1C(F)(F)F]

1-(4-(3,4,5-dichloro-2-oxopyridin-1(2H)-yl)phenyl)-5-(trifluoromethyl)-1H-pyrazole-4-carboxamide (33). 55.7% yield for two steps; ¹H NMR (400 MHz, DMSO-d₆) δ 8.59 (t, J=5.5 Hz, 1H), 8.44 (s, 1H), 8.17 (s, 1H), 7.84-7.57 (m, 4H), 3.32-3.19 (m, 2H), 1.12 (t, J=7.2 Hz, 3H). HRMS (ESI): calcd for C₁₈H₁₁Cl₃F₃N₃O₃ 479.0051 [M+H]⁺, found 479.0050. Purity: 95.9% by HPLC (Rt=3.89 min).

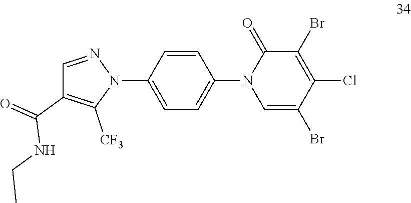 [SMILES: CCNC(=O)c1cnn(-c2ccc(-n3cc(Br)c(Cl)c(Br)c3=O)cc2)c1C(F)(F)F]

1-(4-(3,5-dibromo-4-chloro-2-oxopyridin-1(2H)-yl)phenyl)-5-(trifluoromethyl)-1H-pyrazole-4-carboxamide (34). 48.8% yield for two steps; ¹H NMR (400 MHz, CDCl₃) δ 7.92 (s, 1H), 7.70 (s, 1H), 7.59 (d, J=8.7 Hz, 2H), 7.53 (d, J=8.9 Hz, 2H), 6.01 (t, J=5.1 Hz, 1H), 3.65-3.34 (m, 2H), 1.25 (t, J=7.3 Hz, 3H). HRMS (ESI): calcd for C₁₈H₁₃ClF₄N₄NaO₂ 568.9020 [M+H]⁺, found 568.9033. Purity: 95.8% by HPLC (Rt=3.93 min).

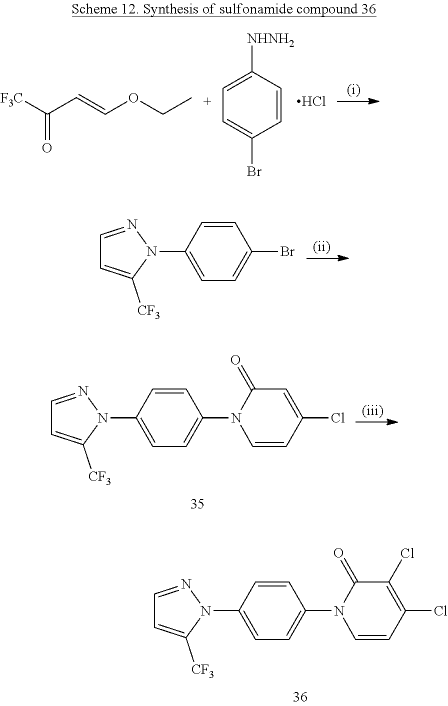

Reagents and conditions: (i) EtOH, 60° C., 6 h; (ii) 4-chloropridinone, CuI, DMCDA, K₂CO₃, toluene, reflux, 12 h; (iii) NCS, DMF, 100° C., 8 h; (d) ClSO₃OH, 160° C., 12 h.

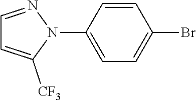 [SMILES: FC(F)(F)c1ccnn1-c1ccc(Br)cc1]

1-(4-bromophenyl)-5-(trifluoromethyl)-1H-pyrazole. (4-bromophenyl)hydrazine hydrochloride (6.7 g, 3 mmol) was dissolved in a solution of ethylamine (3.0 g, 3 mmol) in ethanol (50 mL). Then, 4-ethoxy-1,1,1-trifluorobut-3-en-2-one (5.0 g, 3 mmol) was added, and the reaction solution was reflux for 4 h. After that, the volatiles were removed in vacuo and methylene chloride (30 mL) together with concentrated hydrogen chloride (5 mL) was added. The mixture was stirred at room temperature for 3 h. The organic phase was separated and washed with saturated sodium bicarbonate solution (30 mL). The solution was concentrated, and the residue was purified by column chromatography to afford the desired compounds as a white solid. 89.2% yield; ¹H NMR (400 MHz, CDCl₃) δ 7.93 (d, J=2.0 Hz, 1H), 7.61 (s, 4H), 6.74 (d, J=2.4 Hz, 1H).

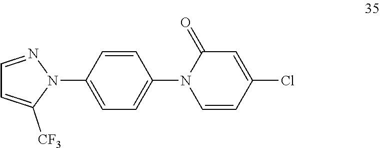 [SMILES: O=c1cc(Cl)ccn1-c1ccc(-n2nccc2C(F)(F)F)cc1]

4-chloro-1-(4-(5-(trifluoromethyl)-1H-pyrazol-1-yl)phenyl)pyridin-2(1H)-one (35). 42.2% yield for two steps; ¹H NMR (400 MHz, CDCl₃) δ 7.75 (d, J=1.2 Hz, 1H), 7.65 (d, J=8.7 Hz, 2H), 7.52 (d, J=8.8 Hz, 2H), 7.33 (d, J=7.4 Hz, 1H), 6.86 (d, J=1.6 Hz, 1H), 6.74 (d, J=2.0 Hz, 1H), 6.34 (dd, J₁=7.4 Hz, J₁=2.3 Hz, 1H).

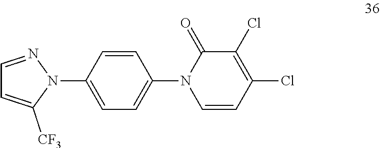 [SMILES: O=c1c(Cl)c(Cl)ccn1-c1ccc(-n2nccc2C(F)(F)F)cc1]

3,4-dichloro-1-(4-(5-(trifluoromethyl)-1H-pyrazol-1-yl)phenyl)pyridin-2(1H)-one (36). 67.6% yield; ¹H NMR (400 MHz, CDCl₃) δ 7.75 (s, 1H), 7.66 (d, J=8.7 Hz, 2H), 7.53 (d, J=8.7 Hz, 2H), 7.30 (d, J=7.5 Hz, 1H), 6.87 (s, 1H), 6.45 (d, J=7.5 Hz, 1H).

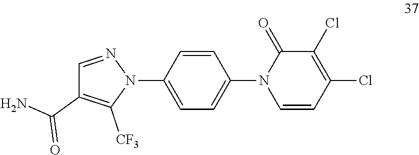 [SMILES: NC(=O)c1cnn(-c2ccc(-n3ccc(Cl)c(Cl)c3=O)cc2)c1C(F)(F)F]

1-(4-(3,4-dichloro-2-oxopyridin-1(2H)-yl)phenyl)-5-(trifluoromethyl)-1H-pyrazole-4-carboxamide (37). 91.4% yield. ¹H NMR (400 MHz, DMSO-d₆) δ 8.19 (s, 1H), 8.03 (s, 1H), 7.87 (d, J=7.4 Hz, 1H), 7.68 (s, 4H), 7.61 (s, 1H), 6.68 (d, J=7.4 Hz, 1H).

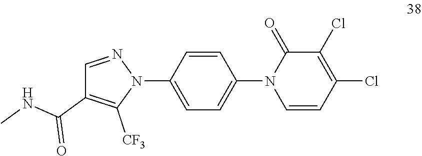 [SMILES: CNC(=O)c1cnn(-c2ccc(-n3ccc(Cl)c(Cl)c3=O)cc2)c1C(F)(F)F]

1-(4-(3,4-dichloro-2-oxopyridin-1(2H)-yl)phenyl)-N-ethyl-5-(trifluoromethyl)-1H-pyrazole-4-carboxamide (38). 84.2% yield. ¹H NMR (400 MHz, DMSO-d₆) δ 8.53 (d, J=4.6 Hz, 1H), 8.15 (s, 1H), 7.88 (d, J=7.4 Hz, 1H), 7.69 (s, 4H), 6.69 (d, J=7.5 Hz, 1H), 2.77 (s, 3H).

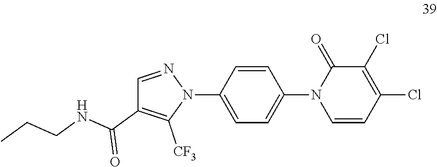 [SMILES: CCCNC(=O)c1cnn(-c2ccc(-n3ccc(Cl)c(Cl)c3=O)cc2)c1C(F)(F)F]

1-(4-(3,4-dichloro-2-oxopyridin-1(2H)-yl)phenyl)-N-propyl-5-(trifluoromethyl)-1H-pyrazole-4-carboxamide (39). 76.8% yield. ¹H NMR (400 MHz, DMSO-d₆) δ 8.59 (t, 1=5.5 Hz, 1H), 8.17 (s, 1H), 7.89 (d, J=7.5 Hz, 1H), 7.71 (d, J=8.7 Hz, 4H), 6.70 (d, J=7.4 Hz, 1H), 3.20 (dd, J₁=13.0 Hz, J₂=6.7 Hz, 2H), 1.51 dt, J=14.5, 7.3 Hz, 2H), 0.90 (t, J=7.4 Hz, 3H).

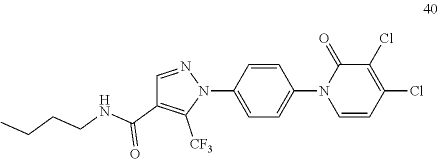 [SMILES: CCCCNC(=O)c1cnn(-c2ccc(-n3ccc(Cl)c(Cl)c3=O)cc2)c1C(F)(F)F]

N-butyl-1-(4-(3,4-dichloro-2-oxopyridin-1(2H)-yl)phenyl)-5-(trifluoromethyl)-1H-pyrazole-4-carboxamide (40). 68.5% yield. ¹H NMR (400 MHz, DMSO-d₆) δ 8.56 (t, J=5.6 Hz, 1H), 8.15 (s, 1H), 7.88 (d, J=7.4 Hz, 1H), 7.68 (s, 4H), 6.69 (d, J=7.4 Hz, 1H), 3.22 (dd, J₁=12.9 Hz, J₂=6.6 Hz, 2H), 1.53-1.41 (m, 2H), 1.33 (dd, J₁=15.1 Hz, J₂=7.6 Hz, 2H), 0.90 (t, J=7.3 Hz, 3H).

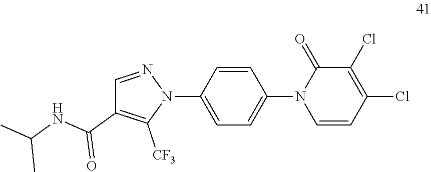 [SMILES: CC(C)NC(=O)c1cnn(-c2ccc(-n3ccc(Cl)c(Cl)c3=O)cc2)c1C(F)(F)F]

1-(4-(3,4-dichloro-2-oxopyridin-1(2H)-yl)phenyl)-N-isopropyl-5-(trifluoromethyl)-1H-pyrazole-4-carboxamide (41). 88.0% yield; ¹H NMR (400 MHz, DMSO-d₆) δ 8.43 (d, J=7.7 Hz, 1H), 8.14 (s, 1H), 7.88 (d, J=7.4 Hz, 1H), 7.73-7.62 (m, 4H), 6.69 (d, J=7.4 Hz, 1H), 4.01 (dt, J₁=13.3, 6.7 Hz, 1H), 1.14 (d, J=6.6 Hz, 6H).

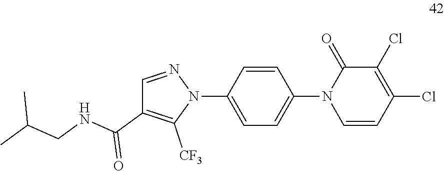 [SMILES: CC(C)CNC(=O)c1cnn(-c2ccc(-n3ccc(Cl)c(Cl)c3=O)cc2)c1C(F)(F)F]

1-(4-(3,4-dichloro-2-oxopyridin-1(2H)-yl)phenyl)-N-isobutyl-5-(trifluoromethyl)-1H-pyrazole-4-carboxamide (42). 78.6% yield; ¹H NMR (400 MHz, DMSO-d₆) δ 8.59 (t, J=5.7 Hz, 1H), 8.16 (s, 1H), 7.88 (d, J=7.5 Hz, 1H), 7.69 (s, 4H), 6.69 (d, 0.1=7.4 Hz, 1H), 3.05 (t, J=6.4 Hz, 2H), 1.80 (dt, J=13.4, 6.7 Hz, 1H), 0.89 (d, J=6.6 Hz, 6H).

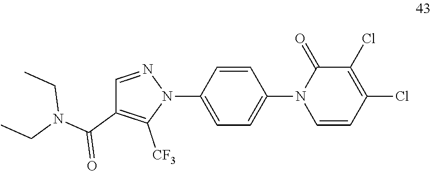 [SMILES: CCN(CC)C(=O)c1cnn(-c2ccc(-n3ccc(Cl)c(Cl)c3=O)cc2)c1C(F)(F)F]

1-(4-(3,4-dichloro-2-oxopyridin-1(2H)-yl)phenyl)-N,N-diethyl-5-(trifluoromethyl)-1H-pyrazole-4-carboxamide (43). 72.8%; ¹H NMR (400 MHz, DMSO-d₆) δ 8.10 (s, 1H), 7.89 (d, J=7.4 Hz, 1H), 7.75 (d, J=8.7 Hz, 2H), 7.69 (d, J=8.6 Hz, 2H), 6.69 (d, J=7.5 Hz, 1H), 3.45 (q, J=7.0 Hz, 2H), 3.28 (q, J=7.0 Hz, 2H), 1.09 (dd, J₁=14.7 Hz, J₂=7.2 Hz, 6H).

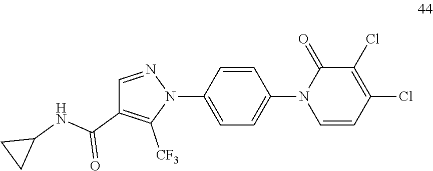 [SMILES: O=C(NC1CC1)c1cnn(-c2ccc(-n3ccc(Cl)c(Cl)c3=O)cc2)c1C(F)(F)F]

N-cyclopropyl-1-(4-(3,4-dichloro-2-oxopyridin-1(2H)-yl)phenyl)-5-(trifluoromethyl)-1H-pyrazole-4-carboxamide (44). 85.6% yield; ¹H NMR (400 MHz, DMSO-d₆) δ 8.62 (d, J=4.3 Hz, 1H), 8.13 (s, 1H), 7.87 (d, J₁=7.5 Hz, 1H), 7.68 (s, 4H), 6.69 (d, J=7.3 Hz, 1H), 2.84-2.75 (m, 1H), 0.71 (q, J=6.0 Hz, 2H), 0.56-0.49 (m, 2H).

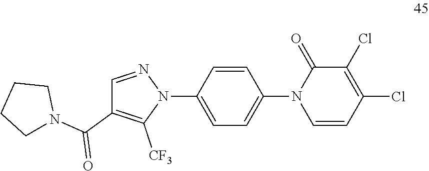 [SMILES: O=C(c1cnn(-c2ccc(-n3ccc(Cl)c(Cl)c3=O)cc2)c1C(F)(F)F)N1CCCC1]

3,4-dichloro-1-(4-(4-(pyrrolidine-1-carbonyl)-5-(trifluoromethyl)-1H-pyrazol-1-yl)phenyl)pyridin-2(1H)-one (45). 75.0% yield; ¹H NMR (400 MHz, CDCl₃) δ 7.80 (s, 1H), 7.64 (d, J=8.7 Hz, 2H), 7.57-7.51 (m, 2H), 7.31 (d, J=7.5 Hz, 1H), 6.47 (d, J=7.5 Hz, 1H), 3.66 (t, J=6.7 Hz, 2H), 3.41 (t, J=6.4 Hz, 2H), 2.05-1.93 (m, 4H).

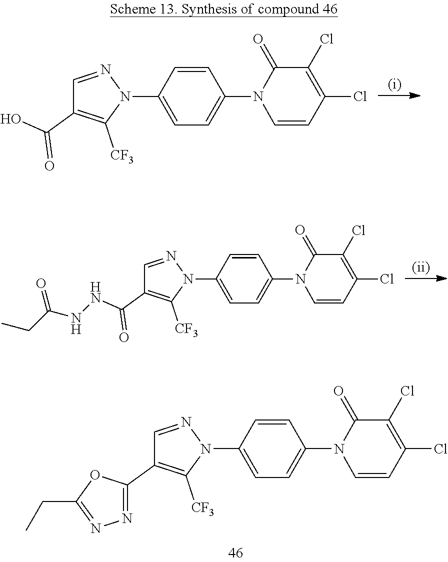 [SMILES: O=C(O)c1cnn(-c2ccc(-n3ccc(Cl)c(Cl)c3=O)cc2)c1C(F)(F)F ; CCC(=O)NNC(=O)c1cnn(-c2ccc(-n3ccc(Cl)c(Cl)c3=O)cc2)c1C(F)(F)F ; CCc1nnc(-c2cnn(-c3ccc(-n4ccc(Cl)c(Cl)c4=O)cc3)c2C(F)(F)F)o1]

Reagents and conditions: (i) propionohydrazide, HBTU, DIPEA, MeCN rt, overnight; (ii) DIPEA, TsCl, rt, 8 h.

3,4-dichloro-1-(4-(4-(5-ethyl-1,3,4-oxadiazol-2-yl)-5-(trifluoromethyl)-1H-pyrazol-1-yl)phenyl)pyridin-2(1H)-one (46). To a mixture of carboxylic acid (105 mg, 0.25 mmol), propionohydrazide (22 mg, 0.25 mmol) and diisopropylethylamine (97 mg, 0.75 mmol) in acetonitrile (5 mL) at room temperature was added HBTU (106 mg, 0.28 mmol) and the resulting mixture was stirred overnight. Diisopropylethylamine (65 mg, 0.5 mmol) was successively added, followed by 4-methylbenzenesulfonyl chloride (143 mg, 0.75 mmol). The resulting reaction mixture was stirred for 8 h, then it was poured in a saturated NaHCO₃solution.

The crude mixture was stirred at room temperature for 20 min, then it was extracted with dichloromethane. The organic solution was washed with water, dried over anhydrous Na₂SO₄, filtered, and concentrated in vacuo. The resulting crude was purified by flash chromatography to afford the product (70.6 mg, 55.1%). ¹H NMR (400 MHz, DMSO-d₆) δ 8.53 (s, 1H), 7.89 (d, J=7.5 Hz, 1H), 7.80 (d, J=8.7 Hz, 2H), 7.72 (d, J=8.8 Hz, 2H), 6.70 (d, J=7.5 Hz, 1H), 2.96 (q, J=7.5 Hz, 2H), 1.32 (t, J=7.6 Hz, 3H).

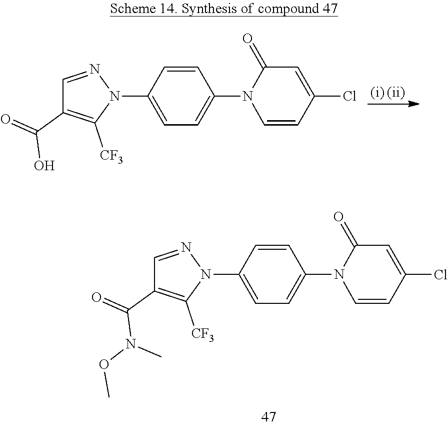 [SMILES: O=C(O)c1cnn(-c2ccc(-n3ccc(Cl)cc3=O)cc2)c1C(F)(F)F ; CON(C)C(=O)c1cnn(-c2ccc(-n3ccc(Cl)cc3=O)cc2)c1C(F)(F)F]

Reagents and conditions: (i) Oxalyl chloride, DMF, CH₂Cl₂, 0° C.-r.t.; (ii) N,O-dimethylhydroxyamine hydrochloride, TEA, CH₂Cl₂, 0° C.-r.t.

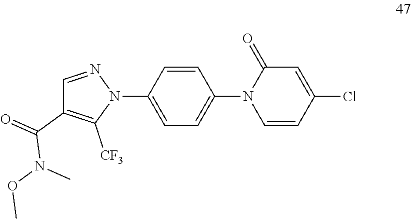 [SMILES: CON(C)C(=O)c1cnn(-c2ccc(-n3ccc(Cl)cc3=O)cc2)c1C(F)(F)F]

1-(4-(4-chloro-2-oxopyridin-1(2H)-yl)phenyl)-N-methoxy-N-methyl-5-(trifluoromethyl)-1H-pyrazole-4-carboxamide (47). A solution of oxalyl chloride (152 mg, 1.2 mmol) in 5 mL CH₂Cl₂ was added dropwise at 0° C. to a solution of 1-(4-(4-chloro-2-oxopyridin-1(2H)-yl)phenyl)-N-methoxy-N-methyl-5-(trifluoromethyl)-1H-pyrazole-4-carboxylic acid (1.92 g, 5 mmol) in CH₂Cl₂ (50 mL)/DMF (0.5 mL). Mixture was allowed to come to room temperature and after stirring for 4 h, N,O-dimethylhydroxylamine hydrochloride (730 mg, 7.5 mmol) in CH₂Cl₂ (20 mL) and TEA (1.01 g, 10 mmol) were then added. The reaction mixture was stirred at room temperature for 5 h. The reaction was quenched by adding water. The organic layer was separated, dried over anhydrous MgSO₄, filtered and concentrated under reduced pressure. The residue was purified by flash column chromatography (silica gel, CH₂Cl₂/MeOH=19/1 v/v) to afford product as a white solid (85.5% yield). ¹H NMR (400 MHz, CDCl₃) δ 7.93 (s, 1H), 7.64 (d, J=8.7 Hz, 2H), 7.54 (d, J=8.7 Hz, 2H), 7.34 (d, J=7.4 Hz, 1H), 6.74 (d, J=2.2 Hz, 1H), 6.35 (dd, J₁=7.4 Hz, J₂=2.2 Hz, 1H), 3.61 (s, 3H), 3.38 (s, 3H).

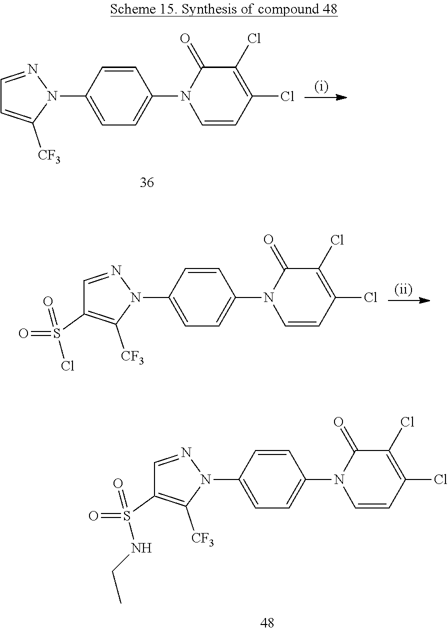 [SMILES: O=c1c(Cl)c(Cl)ccn1-c1ccc(-n2nccc2C(F)(F)F)cc1 ; O=c1c(Cl)c(Cl)ccn1-c1ccc(-n2ncc(S(=O)(=O)Cl)c2C(F)(F)F)cc1 ; CCNS(=O)(=O)c1cnn(-c2ccc(-n3ccc(Cl)c(Cl)c3=O)cc2)c1C(F)(F)F]

Reagents and conditions: (i) ClSO₃OH, 160° C., 12 h; (ii) ethylamine, TEA, THF, r.t., 12 h.

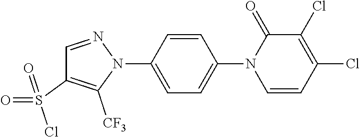 [SMILES: O=c1c(Cl)c(Cl)ccn1-c1ccc(-n2ncc(S(=O)(=O)Cl)c2C(F)(F)F)cc1]

1-(4-(3,4-dichloro-2-oxopyridin-1(2H)-yl)phenyl)-5-(trifluoromethyl)-1H-pyrazole-4-sulfonyl chloride. Compound 36 (374 mg, 1 mmol) was dissolved in chlorosulfonic acid (5 mL) with vigorous stirring at room temperature and heated to 140° C. for 12 h. After cooling to room temperature, the reaction mixture was added dropwise to finely ground ice, filtered at 0° C. and dried under reduced pressure to afford the sulfonyl chloride as pale-yellow solid which was used directly in next step.

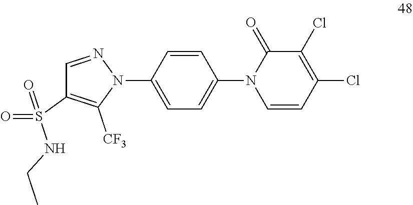 [SMILES: CCNS(=O)(=O)c1cnn(-c2ccc(-n3ccc(Cl)c(Cl)c3=O)cc2)c1C(F)(F)F]

1-(4-(3,4-dichloro-2-oxopyridin-1(2H)-yl)phenyl)-N-ethyl-5-(trifluoromethyl)-1H-pyrazole-4-sulfonamide (48). The title compound was prepared following the general procedure described above for the amide formation using ethylamine as the reactant. 25.3% overall yield in two steps; ¹H NMR (400 MHz, DMSO-d₆) δ 8.19 (s, 1H), 8.04-7.94 (m, 2H), 7.81-7.65 (m, 4H), 7.22 (s, 1H), 3.04-2.88 (m, 2H), 1.05 (t, J=7.2 Hz, 3H). HRMS (ESI): calcd for C₁₇H₁₄Cl₂F₃N₄O₃S 481.0110 [M+H]⁺, found 481.0263. Purity: 97.5% by HPLC (Rt=3.18 min).

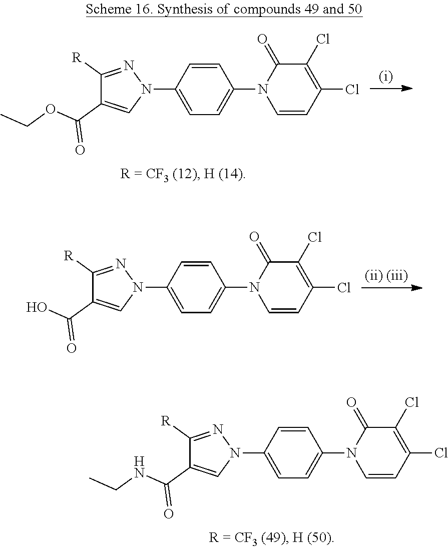

Reagents and conditions: (i) 1 M KOH (aq.), EtOH, H₂O, rt, 8 h; (ii) SOCl₂, CH₂Cl₂, reflux, 4 h; (iii) ethylamine, TEA, THF, r.t., 12 h.

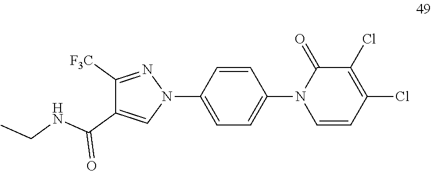 [SMILES: CCNC(=O)c1cn(-c2ccc(-n3ccc(Cl)c(Cl)c3=O)cc2)nc1C(F)(F)F]

1-(4-(3,4-dichloro-2-oxopyridin-1(2H)-yl)phenyl)-N-ethyl-3-(trifluoromethyl)-1H-pyrazole-4-carboxamide (49). 73.0% yield; ¹H NMR (400 MHz, CDCl₃) δ 7.94 (s, 1H), 7.60 (d, J=8.8 Hz, 2H), 7.54 (d, J=8.9 Hz, 2H), 7.30 (d, J=7.5 Hz, 1H), 6.46 (d, J=7.5 Hz, 1H), 5.88 (s, 1H), 3.56-3.45 (m, 2H), 1.26 (t, J=7.3 Hz, 3H).

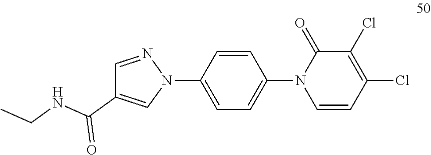 [SMILES: CCNC(=O)c1cnn(-c2ccc(-n3ccc(Cl)c(Cl)c3=O)cc2)c1]

1-(4-(3,4-dichloro-2-oxopyridin-1(2H)-yl)phenyl)-N-ethyl-1H-pyrazole-4-carboxamide (50). 56.9% yield; ¹H NMR (400 MHz, DMSO-d₆) δ 8.98 (s, 1H), 8.26 (t, J=5.5 Hz, 1H), 8.17 (s, 1H), 8.00 (d, J=8.9 Hz, 2H), 7.81 (d, J=7.4 Hz, 1H), 7.61 (d, J=8.9 Hz, 2H), 6.67 (d, J=7.4 Hz, 1H), 3.30-3.20 (m, 2H), 1.12 (t, J=7.2 Hz, 3H).

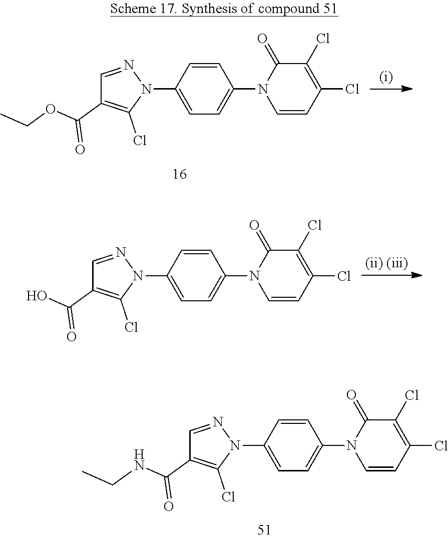 [SMILES: CCOC(=O)c1cnn(-c2ccc(-n3ccc(Cl)c(Cl)c3=O)cc2)c1Cl ; O=C(O)c1cnn(-c2ccc(-n3ccc(Cl)c(Cl)c3=O)cc2)c1Cl ; CCNC(=O)c1cnn(-c2ccc(-n3ccc(Cl)c(Cl)c3=O)cc2)c1Cl]

Reagents and conditions: (i) 1 M KOH (aq.), EtOH, H₂O, rt, 8 h; (ii) SOCl₂, CH₂Cl₂, reflux, 4 h; (iii) ethylamine, TEA, THF, r.t., 12 h.

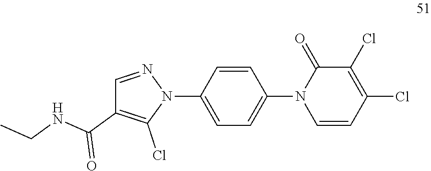 [SMILES: CCNC(=O)c1cnn(-c2ccc(-n3ccc(Cl)c(Cl)c3=O)cc2)c1Cl]

5-chloro-1-(4-(3,4-dichloro-2-oxopyridin-1(2H)-yl)phenyl)-N-ethyl-1H-pyrazole-4-carboxamide (51). 37.2% yield; ¹H NMR (400 MHz, DMSO-d₆) δ 8.66 (t, J=5.2 Hz, 1H), 8.13 (s, 1H), 7.85 (d, J=7.5 Hz, 1H), 7.69 (s, 4H), 6.69 (d, J=7.3 Hz, 1H), 3.25 (q, J=6.7 Hz, 2H), 1.14 (t, J=7.0 Hz, 3H).

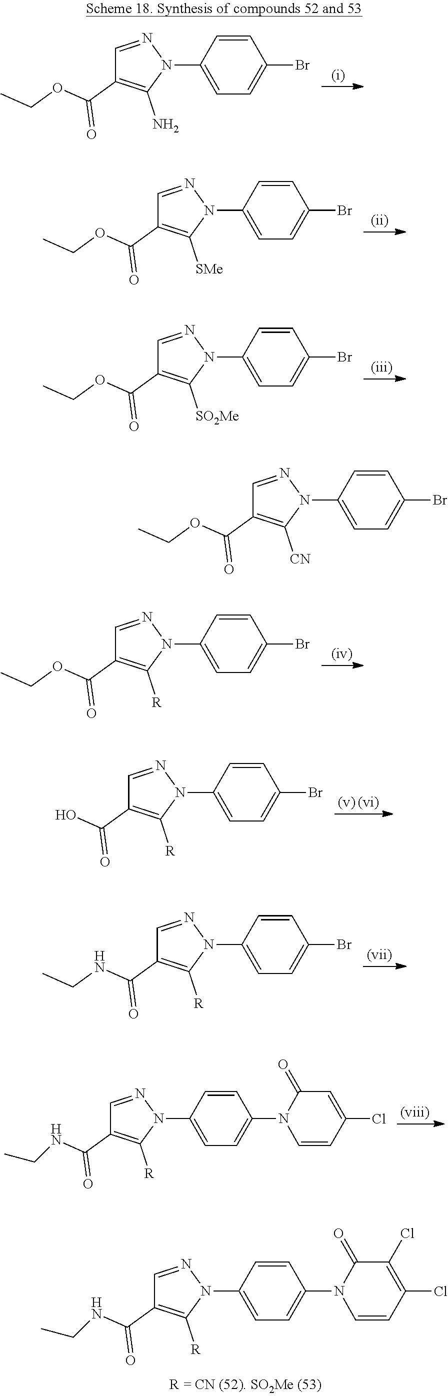

Reagents and conditions: (i) dimethyl disulfide, t-Butyl nitrite, CHCl₃, rt, 16 h; (ii) H₂O₂, AcOH, 100° C., 2 h; (iii) NaCN, DMF, 100° C., 3 h; (iv) 1 M KOH (aq.), EtOH, H₂O, rt, 8 h; (v) SOCl₂, CH₂Cl₂, reflux, 4 h; (vi) ethylamine, TEA, THF, r.t., 12 h; (vii) 4-chloropridinone, CuI, DMCDA, K₂CO₃, toluene, reflux, 12 h; (viii) NCS, DMF, 100° C., 8 h.

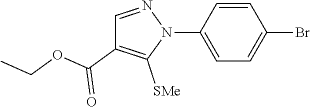 [SMILES: CCOC(=O)c1cnn(-c2ccc(Br)cc2)c1SC]

Ethyl 1-(4-bromophenyl)-5-(methylthio)-1H-pyrazole-4-carboxylate. A solution of ethyl 5-amino-1-(4-bromophenyl)-1H-pyrazole-4-carboxylate (3.1 g, 10 mmol) and dimethyl disulfide (1.88 g, 20 mmol) in chloroform (50 mL) was stirred at room temperature. t-Butyl nitrite (1.55 g, 15 mmol) was added dropwise, and the reaction was stirred at room temperature for 16 h. the solution was washed with water (2×30 mL) and saturated brine (30 mL). the solvent was removed in vacuo and the residue was subjected to silica gel column chromatography (hexanes/ethyl acetate=5/1, v/v) to give 2.32 g (68% yield) of the target product. ¹H NMR (400 MHz, DMSO-d₆) δ 8.19 (s, 1H), 7.78 (d, J=8.6 Hz, 2H), 7.53 (d, J=8.6 Hz, 2H), 4.30 (q, J=7.1 Hz, 2H), 2.39 s 3H), 1.32 t J=7.1 Hz, 3H).

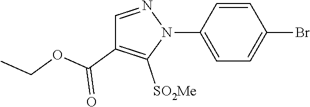 [SMILES: CCOC(=O)c1cnn(-c2ccc(Br)cc2)c1S(C)(=O)=O]

Ethyl 1-(4-bromophenyl)-5-(methylsulfonyl)-1H-pyrazole-4-carboxylate. Ethyl 1-(4-bromophenyl)-5-(methylthio)-1H-pyrazole-4-carboxylate (2.05 g, 6 mmol) was dissolved in acetic acid (30 mL) and 30% hydrogen peroxide was subsequently added. The reaction mixture was stirred at 100° C. for 2 h. After cooling down, the precipitated crystal was filtered, washed with water, and dried in vacuo to obtain 1.52 g (68% yield) of the desired product. ¹H NMR (400 MHz, CDCl₃) δ 8.12 (s, 1H), 7.62 (d, J=8.5 Hz, 2H), 7.29 (d, J=8.5 Hz, 2H), 4.41 (q, J=7.1 Hz, 2H), 3.49 (s, 3H), 1.41 (t, J=7.1 Hz, 3H).

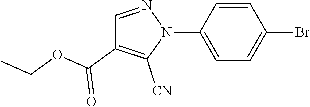 [SMILES: CCOC(=O)c1cnn(-c2ccc(Br)cc2)c1C#N]

Ethyl 1-(4-bromophenyl)-5-cyano-1H-pyrazole-4-carboxylate. A solution containing ethyl 1-(4-bromophenyl)-5-(methylsulfonyl)-1H-pyrazole-4-carboxylate (1.49 g, 4 mmol) and finely ground sodium cyanide powder (490 mg, 10 mmol) in dimethylformamide (20 mL) was stirred at 100° C. for 3 h. The mixture was poured into 100 mL of ice water and extracted with ethyl acetate (3×30 mL). The combined organic layers were removed under reduced pressure and the residue was chromatographed using a silica gel column to yield 1.01 g (79%) of product. ¹H NMR (400 MHz, DMSO-d₆) δ 8.45 (s, 1H), 7.88 (d, J=8.7 Hz, 2H), 7.76 (d, J=8.7 Hz, 2H), 4.36 (q, J=7.1 Hz, 2H), 1.33 (t, J=7.1 Hz, 3H).

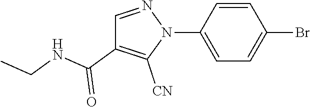 [SMILES: CCNC(=O)c1cnn(-c2ccc(Br)cc2)c1C#N]

1-(4-bromophenyl)-5-cyano-N-ethyl-1H-pyrazole-4-carboxamide. 88.3% yield; the crude compound was used directly in next step without further purification.

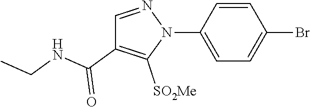 [SMILES: CCNC(=O)c1cnn(-c2ccc(Br)cc2)c1S(C)(=O)=O]

1-(4-bromophenyl)-N-ethyl-5-(methylsulfonyl)-1H-pyrazole-4-carboxamide. 85.7% yield; the crude compound was used directly in next step without further purification.

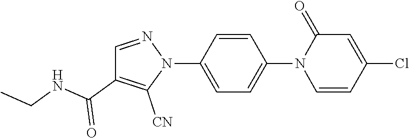 [SMILES: CCNC(=O)c1cnn(-c2ccc(-n3ccc(Cl)cc3=O)cc2)c1C#N]

1-(4-(4-chloro-2-oxopyridin-1(2H)-yl)phenyl)-5-cyano-N-ethyl-1H-pyrazole-4-carboxamide. 52.5% yield; ¹H NMR (400 MHz, DMSO-d₆) δ 8.70 (t, J=5.4 Hz, 1H), 8.45 (s, 1H), 7.89 (dd, J₁=12.4 Hz, J₂=8.0 Hz, 3H), 7.71 (d, J=8.5 Hz, 2H), 6.73 (d, J=1.6 Hz, 1H), 6.51 (dd, J=7.3 Hz, J₂=1.6 Hz, 1H), 3.31 (dt, J=13.4, 6.9 Hz, 2H), 1.15 (t, J=7.2 Hz, 3H).

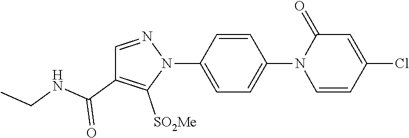 [SMILES: CCNC(=O)c1cnn(-c2ccc(-n3ccc(Cl)cc3=O)cc2)c1S(C)(=O)=O]

1-(4-(4-chloro-2-oxopyridin-1(2H)-yl)phenyl)-N-ethyl-5-(methylsulfonyl)-1H-pyrazole-4-carboxamide. 44.6% yield; ¹H NMR (400 MHz, DMSO-d₆) δ 8.63 (t, J=5.5 Hz, 1H), 8.13 (s, 1H), 7.84 (d, J=7.4 Hz, 1H), 7.69 (d, J=8.6 Hz, 2H), 7.58 (d, J=8.6 Hz, 2H), 6.72 (d, J=2.2 Hz, 1H), 6.49 (dd, J=7.4 Hz, J₂=2.2 Hz, 1H), 3.64 (s, 3H), 3.32-3.19 (m, 2H), 1.13 (t, J=7.2 Hz, 3H).

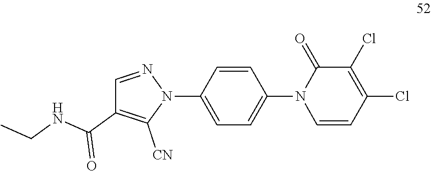 [SMILES: CCNC(=O)c1cnn(-c2ccc(-n3ccc(Cl)c(Cl)c3=O)cc2)c1C#N]

5-cyano-1-(4-(3,4-dichloro-2-oxopyridin-1(2H)-yl)phenyl)-N-ethyl-1H-pyrazole-4-carboxamide (52). 46.0% yield; ¹H NMR (400 MHz, DMSO-d₆) δ 8.69 (t, J=5.5 Hz, 1H), 8.45 (s, 1H), 7.93 (d, J=8.7 Hz, 2H), 7.89 (d, J=7.4 Hz, 1H), 7.75 (d, J=8.7 Hz, 2H), 6.71 (d, J=7.4 Hz, 1H), 3.33-3.27 (m, 2H), 1.15 (t, J=7.2 Hz, 3H).

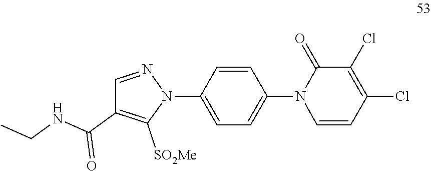 [SMILES: CCNC(=O)c1cnn(-c2ccc(-n3ccc(Cl)c(Cl)c3=O)cc2)c1S(C)(=O)=O]

1-(4-(3,4-dichloro-2-oxopyridin-1(2H)-yl)phenyl)-N-ethyl-5-(methylsulfonyl)-1H-pyrazole-4-carboxamide (53). 38.7% yield; ¹H NMR (400 MHz, DMSO-d⁶) δ 8.64 (t, J=5.5 Hz, 1H), 8.12 (s, 1H), 7.84 (d, J=7.4 Hz, 1H), 7.70 (d, J=8.6 Hz, 2H), 7.58 (d, J=8.5 Hz, 2H), 6.73 (d, J=2.1 Hz, 1H), 3.64 (s, 3H), 3.31-3.17 (m, 2H), 1.13 (t, J=7.2 Hz, 3H).

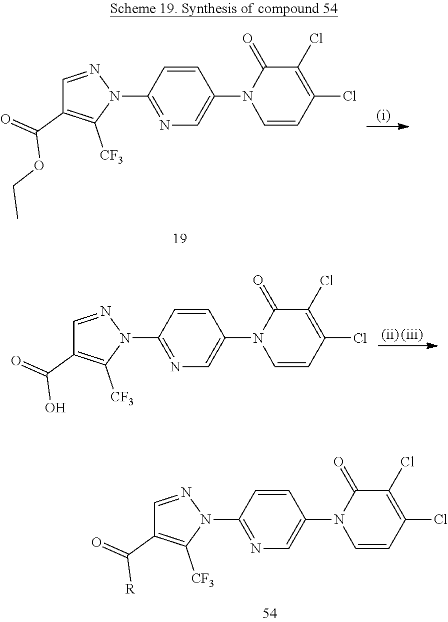

Reagents and conditions: (i) 1 M KOH (aq.), EtOH, H₂O, rt, 8 h; (ii) SOCl₂, DCM, reflux, 4 h; (iii) amines, TEA, THF, r.t., 12 h.

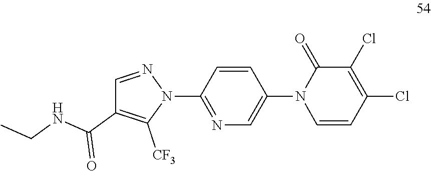 [SMILES: CCNC(=O)c1cnn(-c2ccc(-n3ccc(Cl)c(Cl)c3=O)cn2)c1C(F)(F)F]

1-(3,4-dichloro-2-oxo-2H-[1,3′-bipyridin]-6′-yl)-N-ethyl-5-(trifluoromethyl)-1H-pyrazole-4-carboxamide (54). ¹H NMR (400 MHz, DMSO-d₆) δ 8.68 (d, J=2.5 Hz, 1H), 8.64 (t, J=5.5 Hz, 1H), 8.25 (dd, J₁=8.6 Hz, J₂=2.5 Hz, 1H), 8.20 (s, 1H), 7.99 (dd, J₁=8.0 Hz, J₂=6.8 Hz, 2H), 6.66 (d, J=7.9 Hz, 1H), 3.31-3.21 (m, 2H), 1.12 (t, J=7.2 Hz, 3H).

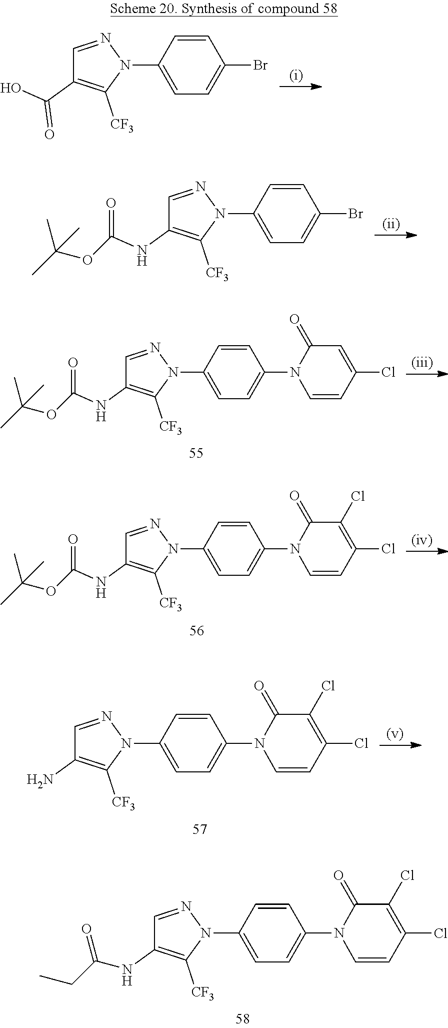 [SMILES: O=C(O)c1cnn(-c2ccc(Br)cc2)c1C(F)(F)F ; CC(C)(C)OC(=O)Nc1cnn(-c2ccc(Br)cc2)c1C(F)(F)F ; CC(C)(C)OC(=O)Nc1cnn(-c2ccc(-n3ccc(Cl)cc3=O)cc2)c1C(F)(F)F ; CC(C)(C)OC(=O)Nc1cnn(-c2ccc(-n3ccc(Cl)c(Cl)c3=O)cc2)c1C(F)(F)F ; Nc1cnn(-c2ccc(-n3ccc(Cl)c(Cl)c3=O)cc2)c1C(F)(F)F ; CCC(=O)Nc1cnn(-c2ccc(-n3ccc(Cl)c(Cl)c3=O)cc2)c1C(F)(F)F]

Reagents and conditions: (i) DPPA, TEA, t-BuOH, reflux, 2 h; (ii) 4-chloropridinone, CuI, DMCDA, K₂CO₃, toluene, reflux, 12 h; (iii) NCS, DMF, 100° C., 8 h; (iv) TFA, CH₂Cl₂, rt, 2 h; (v) propionyl chloride, TEA, rt, 4 h.

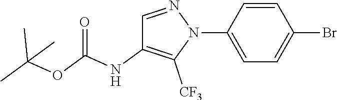 [SMILES: CC(C)(C)OC(=O)Nc1cnn(-c2ccc(Br)cc2)c1C(F)(F)F]

Tert-butyl (1-(4-bromophenyl)-5-(trifluoromethyl)-1H-pyrazol-4-yl)carbamate. Diphenylphosphoryl azide (5.5 g, 20 mmol) and triethylamine (2.02 g, 20 mmol) were added dropwise to a solution of 1-(4-bromophenyl)-5-(trifluoromethyl)-1H-pyrazole-4-carboxylic acid (6.7 g, 20 mmol) in tert-butanol (150 mL). The mixture was stirred and heated to reflux for 2 h. After cooling to room temperature, the solvent was removed under reduced pressure. Purification by silica gel column chromatography gave the title compound (4.68 g, 57.7%) as a pale-yellow solid. ¹H NMR (400 MHz, CDCl₃) δ 8.27 (s, 1H), 7.61 (d, J=8.7 Hz, 2H), 7.32 (d, J=8.6 Hz, 2H), 6.54 (s, 1H), 1.54 (s, 9H).

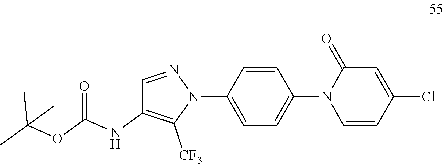 [SMILES: CC(C)(C)OC(=O)Nc1cnn(-c2ccc(-n3ccc(Cl)cc3=O)cc2)c1C(F)(F)F]

Tert-butyl (1-(4-(4-chloro-2-oxopyridin-1(2H)-yl)phenyl)-5-(trifluoromethyl)-1H-pyrazol-4-yl)carbamate (55). 29.8% yield; ¹H NMR (400 MHz, DMSO-d₆) δ 9.17 (s, 1H), 7.93 (s, 1H), 7.87 (d, J=7.4 Hz, 1H), 6.72 (d, J=2.2 Hz, 1H), 6.50 (dd, J₁=7.4 Hz, J₂=2.2 Hz, 1H), 1.47 (s, 9H).

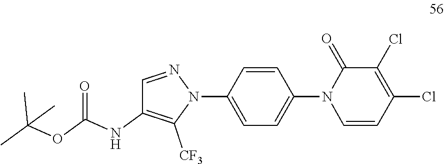 [SMILES: CC(C)(C)OC(=O)Nc1cnn(-c2ccc(-n3ccc(Cl)c(Cl)c3=O)cc2)c1C(F)(F)F]

Tert-butyl (1-(4-(3,4-dichloro-2-oxopyridin-1(2H)-yl)phenyl)-5-(trifluoromethyl)-1H-pyrazol-4-yl)carbamate (56). 36.3% yield; ¹H NMR (400 MHz, CDCl₃) δ 7.60 (d, J=8.6 Hz, 2H), 7.50 (d, J=8.7 Hz, 2H), 7.29 (t, J=5.2 Hz, 1H), 7.27 (s, OH), 6.43 (dd, J₁=17.2 Hz, J₂=4.9 Hz, 1H), 1.55 (s, 9H).

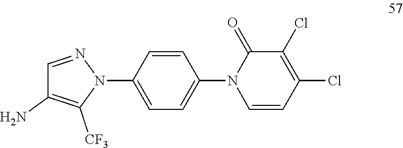 [SMILES: Nc1cnn(-c2ccc(-n3ccc(Cl)c(Cl)c3=O)cc2)c1C(F)(F)F]

1-(4-(4-amino-5-(trifluoromethyl)-1H-pyrazol-1-yl)phenyl)-3,4-dichloropyridin-2(1H)-one (57). Trifluoroacetic acid (3 mL) was added dropwise to a solution of tert-butyl (1-(4-(3,4-dichloro-2-oxopyridin-1(2H)-yl)phenyl)-5-(trifluoromethyl)-1H-pyrazol-4-yl)carbamate (978.6 mg, 2 mmol) in methylene chloride (3 mL). After stirring at room temperature for 2 h, the reaction mixture was concentrated in vacuo and the residue was subjected to the silica gel column chromatography to afford desired product as white solid (420.3 mg, 54.0% yield). ¹H NMR (400 MHz, CDCl₃) δ 7.60 (d, J=8.6 Hz, 2H), 7.47 (d, J=8.7 Hz, 2H), 7.38 (s, 1H), 7.29 (d, J=7.5 Hz, 1H), 6.44 (d, J=7.5 Hz, 1H), 3.57 (s, 2H).

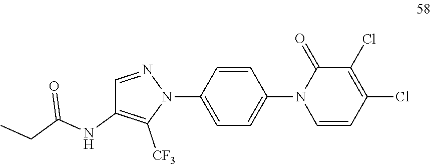 [SMILES: CCC(=O)Nc1cnn(-c2ccc(-n3ccc(Cl)c(Cl)c3=O)cc2)c1C(F)(F)F]

N-(1-(4-(3,4-dichloro-2-oxopyridin-1(2H)-yl)phenyl)-5-(trifluoromethyl)-1H-pyrazol-4-yl)propionamide (58). 84.7% yield; ¹H NMR (400 MHz, DMSO-d₆) δ 9.78 (s, 1H), 8.08 (s, 1H), 7.89 (d, J=7.5 Hz, 1H), 7.68 (s, 4H), 6.69 (d, J=7.4 Hz, 1H), 2.37 (q, J=7.5 Hz, 2H), 1.09 (t, J=7.5 Hz, 3H).

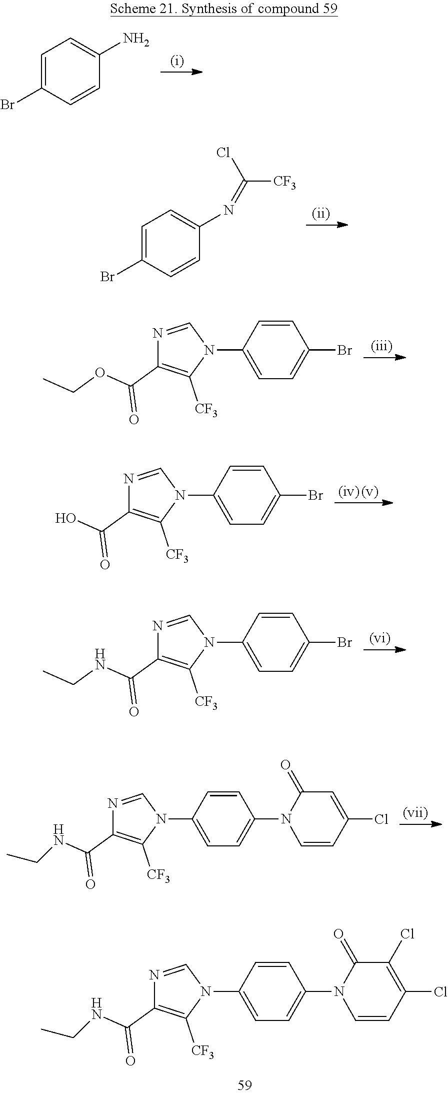

Reagents and conditions: (i) PPh₃, TEA, TFA, CCl₄, reflux, 5 h; (ii) t-BuOK, DMF, 60° C., 1 h; (iii) 1 M KOH (aq.), EtOH, H₂O, rt, 8 h; (iv) SOCl₂, CH₂Cl₂, reflux for 4 h; (v) ethylamine, TEA, THF, r.t., 12 h; (vi) 4-chloropridinone, CuI, DMCDA, K₂CO₃, toluene, reflux, 12 h; (vii) NCS, DMF, 100° C., 8 h.

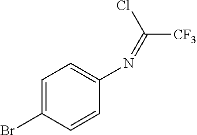 [SMILES: FC(F)(F)/C(Cl)=N/c1ccc(Br)cc1]

N-(4-bromophenyl)-2,2,2-trifluoroacetimidoyl chloride. A 100 mL two-necked flask equipped with a septum cap, a condenser, and a Teflon-coated magnetic stir bar was charged with triphenylphosphine (16.52 g, 63 mmol), triethylamine (3.51 mL, 25.2 mmol), trifluoroacetic acid (1.61 mL, 21 mmol), and carbon tetrachloride (30 mL). After stirring for 10 min at 0° C., a solution of 4-bromoaniline (3.61 g, 21 mmol) dissolved in carbon tetrachloride (30.0 mL) was added. The mixture was then refluxed for 5 h. After cooling down to room temperature, the mixture was diluted with hexanes (150 mL) and then filtered. The solid was washed with hexanes several times. The filtrate was concentrated under reduced pressure and the residue was purified by flash chromatography on silica gel to obtain the desired product (3.19 g, 53.0%). ¹H NMR (400 MHz, CDCl₃) δ 7.57 (d, J=8.8 Hz, 2H), 7.00 (d, J=8.7 Hz, 2H). ¹⁹F NMR (376 MHz, CDCl₃) δ −75.67.

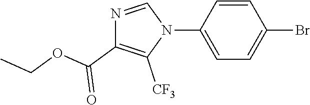 [SMILES: CCOC(=O)c1ncn(-c2ccc(Br)cc2)c1C(F)(F)F]

Ethyl 1-(4-bromophenyl)-5-(trifluoromethyl)-1H-imidazole-4-carboxylate. t-BuOK (1.35 g, 12 mmol) was added to a solution of N-(4-bromophenyl)-2,2,2-trifluoroacetimidoyl chloride (4.3 g, 15 mmol) and ethyl 2-isocyanoacetate (1.13 g, 10 mmol) in DMF (20 mL). After stirring at 60° C. for 1 h, the reaction mixture was cooled down and quenched by water and extracted with ethyl acetate (3×50 mL). The combined organic layers were dried over anhydrous Na₂SO₄, and the solvent was removed in vacuo and the residue was purified by column chromatography on silica gel to afford the imidazole product (2.25 g, 62.3% yield). ¹H NMR (CDCl₃, 400 MHz) δ 7.58 (s, 1H), 7.69 (d, 2H, J=8.4 Hz), 7.50 (d, 2H, J=8.2 Hz), 4.32 (q, 2H, J=6.8 Hz), 1.39 (t, 3H, J=7.0 Hz.

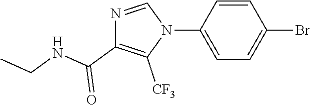 [SMILES: CCNC(=O)c1ncn(-c2ccc(Br)cc2)c1C(F)(F)F]

1-(4-bromophenyl)-N-ethyl-5-(trifluoromethyl)-1H-imidazole-4-carboxamide (74.5% yield). The crude compound was used directly in next step without further purification.

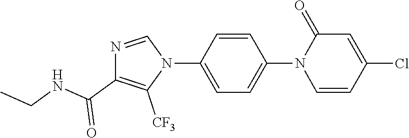 [SMILES: CCNC(=O)c1ncn(-c2ccc(-n3ccc(Cl)cc3=O)cc2)c1C(F)(F)F]

1-(4-(4-chloro-2-oxopyridin-1(2H)-yl)phenyl)-N-ethyl-5-(trifluoromethyl)-1H-imidazole-4-carboxamide. 54.7% yield; ¹H NMR (400 MHz, DMSO-d₆) δ 8.50 (t, J=5.9 Hz, 1H), 8.24 (s, 1H), 7.84 (d, J=7.4 Hz, 1H), 7.68 (q, J=8.8 Hz, 4H), 6.72 (d, J=2.1 Hz, 1H), 6.50 (dd, J₁=7.4 Hz, J₂=2.1 Hz, 1H), 3.32-3.23 (m, 2H), 1.11 (t, J=7.2 Hz, 3H).

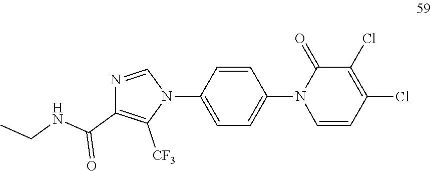 [SMILES: CCNC(=O)c1ncn(-c2ccc(-n3ccc(Cl)c(Cl)c3=O)cc2)c1C(F)(F)F]

1-(4-(3,4-dichloro-2-oxopyridin-1(2H)-yl)phenyl)-N-ethyl-5-(trifluoromethyl)-1H-imidazole-4-carboxamide (59). 44.0% yield; ¹H NMR (400 MHz, CDCl₃) δ 7.63-7.45 (m, 6H), 7.33 (d, J=7.5 Hz, 1H), 6.49 (d, J=7.5 Hz, 1H), 3.58-3.37 (m, 2H), 1.34-1.19 (m, 3H).

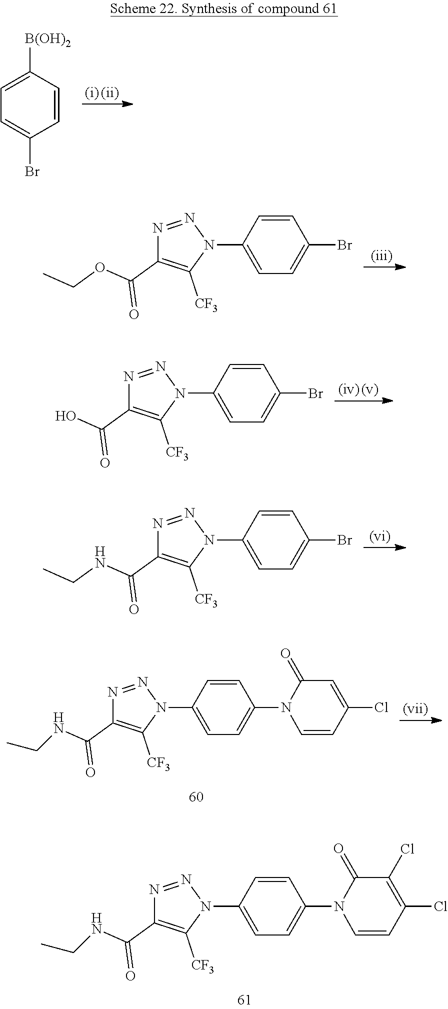 [SMILES: OB(O)c1ccc(Br)cc1 ; CCOC(=O)c1nnn(-c2ccc(Br)cc2)c1C(F)(F)F ; O=C(O)c1nnn(-c2ccc(Br)cc2)c1C(F)(F)F ; CCNC(=O)c1nnn(-c2ccc(Br)cc2)c1C(F)(F)F ; CCNC(=O)c1nnn(-c2ccc(-n3ccc(Cl)cc3=O)cc2)c1C(F)(F)F ; CCNC(=O)c1nnn(-c2ccc(-n3ccc(Cl)c(Cl)c3=O)cc2)c1C(F)(F)F]

Reagents and conditions: (i) NaN₃, Cu(OAc)₂, DMSO/H₂O, rt; (ii) ethyl 4,4,4-trifluoro-3-oxobutanoate, piperidine, 80° C., overnight; (iii) 1 M KOH (aq.), EtOH, H₂O, rt, 8 h; (iv) SOCl₂, CH₂Cl₂, reflux, 4 h; (v) ethylamine, TEA, THF, r.t., 12 h; (vi) 4-chloropridinone, CuI, DMCDA, K₂CO₃, toluene, reflux, 12 h; (vii) NCS, DMF, 100° C., 8 h.

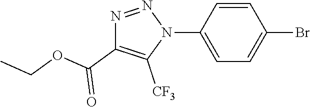 [SMILES: CCOC(=O)c1nnn(-c2ccc(Br)cc2)c1C(F)(F)F]

Ethyl 1-(4-bromophenyl)-5-(trifluoromethyl)-1H-1,2,3-triazole-4-carboxylate. To a stirred solution of (4-bromophenyl)boronic acid (2.01 g, 12 mmol) in 33 mL DMSO/H₂O (10:1, v/v), sodium azide (1.56 g, 24 mmol), Cu(OAc)₂ (218 mg, 1.2 mmol) were added successively. The mixture was stirred for 2 h at room temperature. Upon the completion of conversion, ethyl 4,4,4-trifluoro-3-oxobutanoate (1.84 g, 10 mmol), piperidine (204 mg, 2.4 mmol) were added to the solution. The reaction mixture was heated to 80° C. and stirred overnight. Then, the reaction was quenched by the addition of water (100 mL). The resulting suspension was filtered, and the filter cake was washed with ethyl acetate. The organic phase of the filtrate was separated, and the aqueous phase was extracted with additional ethyl acetate (2×50 mL). The combined organic layers were dried over anhydrous Na2SO₄, concentrated under reduced pressure to leave the crude product, which was purified by flash chromatography to afford the product (2.8 g, 77.1% yield). ¹H NMR (400 MHz, CDCl₃) δ 7.73 (d, J=8.5 Hz, 2H), 7.36 (d, J=8.5 Hz, 2H), 4.51 (d, J=7.1 Hz, 2H), 1.45 (t, J=7.1 Hz, 3H).

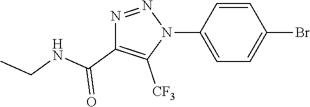 [SMILES: CCNC(=O)c1nnn(-c2ccc(Br)cc2)c1C(F)(F)F]

1-(4-bromophenyl)-N-ethyl-5-(trifluoromethyl)-1H-1,2,3-triazole-4-carboxamide (79.0% yield). The crude compound was used directly in next step without further purification.

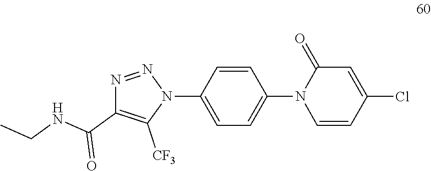 [SMILES: CCNC(=O)c1nnn(-c2ccc(-n3ccc(Cl)cc3=O)cc2)c1C(F)(F)F]

1-(4-(4-chloro-2-oxopyridin-1(2H)-yl)phenyl)-N-ethyl-5-(trifluoromethyl)-1H-1,2,3-triazole-4-carboxamide (60). 36.8% yield; ¹H NMR (400 MHz, CDCl₃) δ 7.63 (s, 4H), 7.35 (d, J=7.4 Hz, 1H), 6.76 (d, J=1.9 Hz, 1H), 6.37 (dd, J=7.4 Hz, J₂=1.9 Hz, 1H), 3.61-3.50 (m, 2H), 1.30 (t, J=7.3 Hz, 3H).

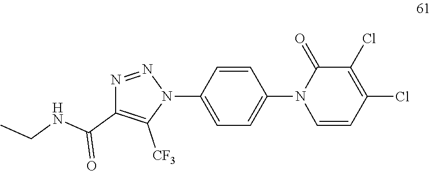 [SMILES: CCNC(=O)c1nnn(-c2ccc(-n3ccc(Cl)c(Cl)c3=O)cc2)c1C(F)(F)F]

1-(4-(3,4-dichloro-2-oxopyridin-1(2H)-yl)phenyl)-N-ethyl-5-(trifluoromethyl)-1H-1,2,3-triazole-4-carboxamide (61). 30.7% yield; ¹H NMR (400 MHz, DMSO-d₆) δ 9.04 (t, J=5.7 Hz, 1H), 7.96-7.85 (m, 3H), 7.77 (d, J=8.6 Hz, 2H), 6.71 (d, J=7.4 Hz, 1H), 3.34-3.27 (m, 2H), 1.13 (t, J=7.2 Hz, 3H).

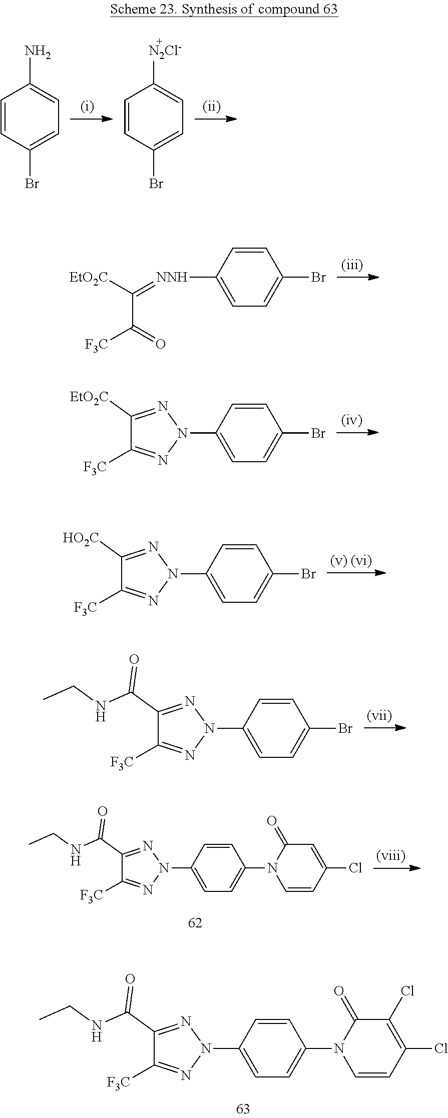

Reagents and conditions: (i) HCl (6 M), NaNO₂, 0° C., 1 h; (ii) ethyl 4,4,4-trifluoro-3-oxobutanoate, EtOH, 0° C., 30 min, rt, 2 h; (iii) NH₄Ac, CuBr₂, DMF/AcOH, air, 110° C., 6 h; (iv) 1 M KOH (aq.), EtOH, H₂O, rt, 8 h; (v) SOCl₂, CH₂Cl₂, reflux, 4 h; (vi) ethylamine, TEA, THF, r.t., 12 h; (vii) 4-chloropridinone, CuI, DMCDA, K₂CO₃, toluene, reflux, 12 h; (viii) NCS, DMF, 100° C., 8 h.

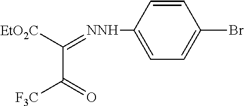

Ethyl 2-(2-(4-bromophenyl)hydrazono)-4,4,4-trifluoro-3-oxobutanoate. 4-bromoaniline (1.72 g, 10 mmol) was dissolved in hydrochloric acid (6 M, 30 mL) at 60° C. After cooling down to 0° C., the solution of sodium nitrite (759 mg, 11 mmol) in water (10 mL) was added dropwise over 30 min to maintain the temperature of the reaction below 5° C. The resulting diazonium salt solution was stirred additional 30 min. A solution of sodium acetate (6.0 g) in water (10 mL) was added to a solution of ethyl 4,4,4-trifluoro-3-oxobutanoate (1.84 g, 10 mmol) in ethanol (40 mL). The mixture was cooled to 0° C., and the diazonium salt solution was added dropwise over 20 min. The resulting mixture was stirred at 0° C. for 30 min and allowed to stir at room temperature for 2 h. The aqueous mixture was extracted with EtOAc (3×30 mL). The combined organic layers were dried over Na₂SO₄ and concentrated under reduced pressure. The crude product was further purified by silica gel column chromatography to give the desired arylhydrazonoketone product (1.76 g, 48.4%). ¹H NMR (400 MHz, CDCl₃) δ 13.45 (s, 1H), 7.56 (d, J=8.8 Hz, 2H), 7.30 (d, J=8.9 Hz, 2H), 4.41 (q, J=7.1 Hz, 2H), 1.42 (t, J=7.1 Hz, 3H).

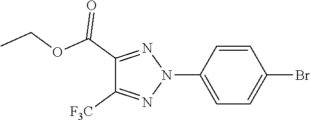 [SMILES: CCOC(=O)c1nn(-c2ccc(Br)cc2)nc1C(F)(F)F]

Ethyl 2-(4-bromophenyl)-5-(trifluoromethyl)-2H-1,2,3-triazole-4-carboxylate. To a solution of ethyl 2-(2-(4-bromophenyl)hydrazono)-4,4,4-trifluoro-3-oxobutanoate (1.47 g, 4.0 mmol) in DMF/HOAc (30 mL, 1/1, v/v) were added ammonium acetate (2.46 g, 32.0 mmol) and copper bromide (89.3 mg, 0.4 mmol). The reaction mixture was heated at 110° C. for 6 h with air continuously pumped into the solution. The mixture was cooled to room temperature and quenched with water (150 mL). After extracted with ethyl acetate (3×40 mL), The organic phase was combined and dried over anhydrous Na₂SO₄. The solvent was removed under reduced pressure and the crude reaction mixture was purified by flash silica gel chromatography to give the desired products (732 mg, 50.3%). ¹H NMR (400 MHz, CDCl₃) δ 8.05 (d, J=8.7 Hz, 2H), 7.67 (d, J=8.7 Hz, 2H), 4.50 (q, J=7.1 Hz, 2H), 1.45 (t, J=7.1 Hz, 3H).

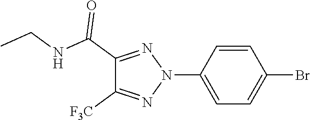 [SMILES: CCNC(=O)c1nn(-c2ccc(Br)cc2)nc1C(F)(F)F]

2-(4-bromophenyl)-N-ethyl-5-(trifluoromethyl)-2H-1,2,3-triazole-4-carboxamide. 89.0% yield; the crude compound was used directly in next step without further purification.

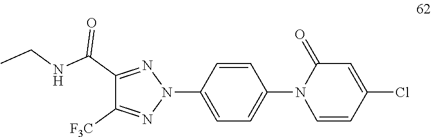 [SMILES: CCNC(=O)c1nn(-c2ccc(-n3ccc(Cl)cc3=O)cc2)nc1C(F)(F)F]

2-(4-(4-chloro-2-oxopyridin-1(2H)-yl)phenyl)-N-ethyl-5-(trifluoromethyl)-2H-1,2,3-triazole-4-carboxamide (62). 57.5% yield; ¹H NMR (400 MHz, CDCl₃) δ 8.25 (d, J=8.9 Hz, 2H), 7.58 (d, J=8.9 Hz, 2H), 7.33 (d, J=7.4 Hz, 1H), 6.85 (s, 1H), 6.74 (d, J=1.8 Hz, 1H), 6.36 (dd, J₁=7.4 Hz, J₂=2.2 Hz, 1H), 3.64-3.46 (m, 2H), 1.31 (t, J=7.3 Hz, 3H).

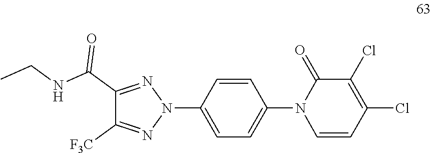 [SMILES: CCNC(=O)c1nn(-c2ccc(-n3ccc(Cl)c(Cl)c3=O)cc2)nc1C(F)(F)F]

2-(4-(3,4-dichloro-2-oxopyridin-1(2H)-yl)phenyl)-N-ethyl-5-(trifluoromethyl)-2H-1,2,3-triazole-4-carboxamide (63). 36.4% yield; ¹H NMR (400 MHz, DMSO-d₆) δ 8.98 (d, J=5.5 Hz, 1H), 8.24 (d, J=8.8 Hz, 2H), 7.86 (d, J=7.4 Hz, 1H), 7.78 (d, J=8.8 Hz, 2H), 6.71 (d, J=7.4 Hz, 1H), 3.33-3.29 (m, 2H), 1.15 (t, J=7.2 Hz, 3H).

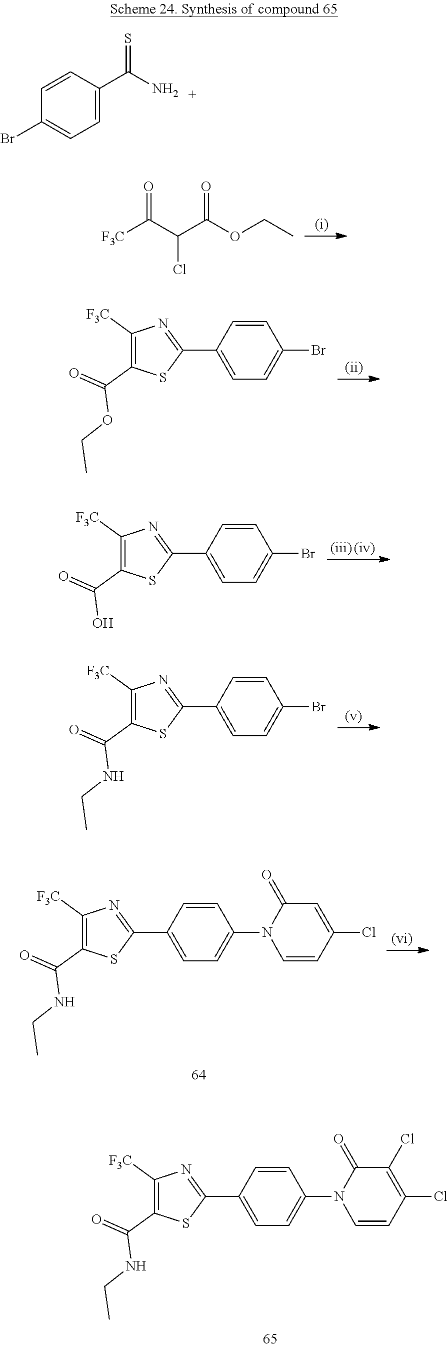

Reagents and conditions: (i) EtOH, reflux; (ii) 1 M KOH (aq.), EtOH, H2O, rt, 8 h; (iii) SOCl2, CH2Cl2, reflux, 4 h; (iv) ethylamine, TEA, THF, r.t., 12 h; (v) 4-chloropridinone, CuI, DMCDA, K2CO3, toluene, reflux, 12 h; (vi) NCS, DMF, 100° C., 8 h.

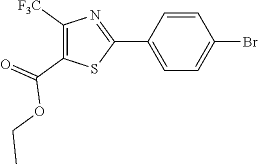 [SMILES: CCOC(=O)c1sc(-c2ccc(Br)cc2)nc1C(F)(F)F]

Ethyl 2-(4-bromophenyl)-4-(trifluoromethyl)thiazole-5-carboxylate. 4-bromobenzothioamide (1.08 g, 5 mmol) was dissolved in anhydrous ethanol at 0° C. Ethyl 2-chloro-4,4,4-trifluoro-3-oxobutanoate (1.09 g, 5 mmol) was added dropwise over 10 min. The reaction was heated up to reflux for 24 h with stirring. After that, the solvent was removed under vacuo and the residue was subjected to flash chromatography to give the product (3.23 g, 85.8% yield). ¹H NMR (400 MHz, CDCl₃) δ 7.74 (t, J=6.7 Hz, 2H), 7.60 (d, J=8.5 Hz, 2H), 4.38-4.24 (m, 2H), 1.34 (t, J=7.1 Hz, 3H).

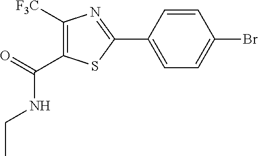 [SMILES: CCNC(=O)c1sc(-c2ccc(Br)cc2)nc1C(F)(F)F]

2-(4-bromophenyl)-N-ethyl-4-(trifluoromethyl)thiazole-5-carboxamide. 89.0% yield; the crude compound was used directly in next step without further purification.

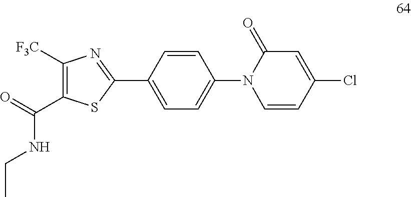 [SMILES: CCNC(=O)c1sc(-c2ccc(-n3ccc(Cl)cc3=O)cc2)nc1C(F)(F)F]

2-(4-(4-chloro-2-oxopyridin-1(2H)-yl)phenyl)-N-ethyl-4-(trifluoromethyl)thiazole-5-carboxamide. 59.3% yield; ¹H NMR (400 MHz, CDCl₃) δ 8.07 (d, J=8.3 Hz, 2H), 7.50 (d, J=8.3 Hz, 2H), 7.31 (d, J=7.4 Hz, 1H), 6.72 (d, J=1.8 Hz, 1H), 6.34 (dd, J=7.3 Hz, J₂=1.9 Hz, 1H), 6.26 (s, 1H), 3.61-3.45 (m, 2H), 1.28 (t, J=7.2 Hz, 3H).

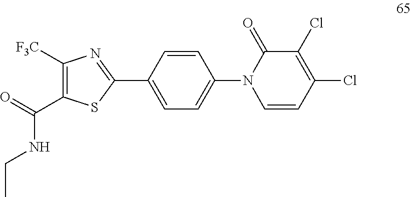 [SMILES: CCNC(=O)c1sc(-c2ccc(-n3ccc(Cl)c(Cl)c3=O)cc2)nc1C(F)(F)F]

2-(4-(3,4-dichloro-2-oxopyridin-1(2H)-yl)phenyl)-N-ethyl-4-(trifluoromethyl)thiazole-5-carboxamide. 41.4% yield; ¹H NMR (400 MHz, DMSO-d₆) δ 9.04 (t, J=5.2 Hz, 1H), 8.15 (d, J=8.5 Hz, 2H), 7.83 (d, J=7.5 Hz, 1H), 7.67 (d, J=8.4 Hz, 2H), 6.70 (d, J=7.5 Hz, 1H), 3.26 (q, J=6.7 Hz, 2H), 1.11 (t, J=7.2 Hz, 3H).

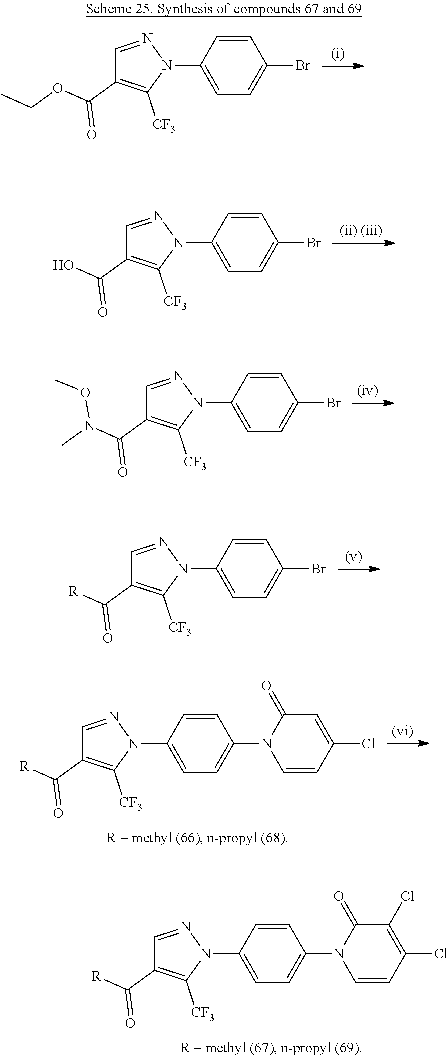

Reagents and conditions: (i) 1 M KOH (aq.), EtOH, H₂O, rt, 8 h; (ii) SOCl₂, CH₂Cl₂, reflux, 4 h; (iii) N,O-dimethylhydroxyamine hydrochloride, TEA, CH₂Cl₂, 0° C.-r.t.; (iv) MeMgBr or n-PrMgBr, THF, 0° C.-r.t; (v) 4-chloropridinone, CuI, DMCDA, K₂CO₃, toluene, reflux, 12 h; (iv) NCS, DMF, 100° C., 8 h.

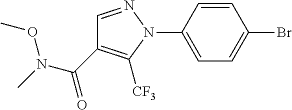 [SMILES: CON(C)C(=O)c1cnn(-c2ccc(Br)cc2)c1C(F)(F)F]

1-(4-bromophenyl)-N-methoxy-N-methyl-5-(trifluoromethyl)-1H-pyrazole-4-carboxamide. 86.9% yield; ¹H NMR (400 MHz, CDCl₃) δ 7.97 (s, 1H), 7.84-7.79 (m, 4H), 3.74 (s, 3H), 3.34 (s, 3H).

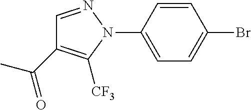 [SMILES: CC(=O)c1cnn(-c2ccc(Br)cc2)c1C(F)(F)F]

1-(1-(4-bromophenyl)-5-(trifluoromethyl)-1H-pyrazol-4-yl)ethenone. 55.4 yield; ¹H NMR (400 MHz, CDCl₃) δ 8.12 (d, J=1.9 Hz, 1H), 7.71-7.66 (m, 2H), 7.35-7.31 (m, 2H), 2.64 (s, 3H).

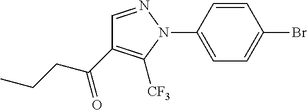 [SMILES: CCCC(=O)c1cnn(-c2ccc(Br)cc2)c1C(F)(F)F]

1-(1-(4-bromophenyl)-5-(trifluoromethyl)-1H-pyrazol-4-yl)butan-1-one. 63.8% yield; ¹H NMR (400 MHz, CDCl₃) δ 8.04 (s, 1H), 7.65 (d, J=8.5 Hz, 2H), 7.32 (d, J=8.6 Hz, 2H), 2.85 (t, J=7.2 Hz, 2H), 1.78 (dd, J₁=14.7 Hz, J₂=7.3 Hz, 2H), 1.01 (t, J=7.4 Hz, 3H).

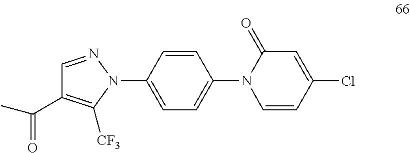 [SMILES: CC(=O)c1cnn(-c2ccc(-n3ccc(Cl)cc3=O)cc2)c1C(F)(F)F]

1-(4-(4-acetyl-5-(trifluoromethyl)-1H-pyrazol-1-yl)phenyl)-4-chloropyridin-2(1H)-one (66). 66.8 yield; ¹H NMR (400 MHz, CDCl₃) δ 8.09 (s, 1H), 7.56 (q, J=8.9 Hz, 4H), 7.33 (d, J=7.4 Hz, 1H), 6.74 (d, J=2.0 Hz, 1H), 6.35 (dd, J=7.4 Hz, J₂=2.1 Hz, 1H), 2.59 (s, 3H).

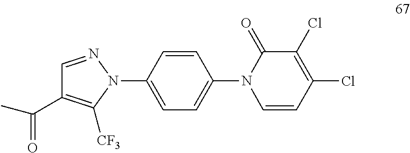 [SMILES: CC(=O)c1cnn(-c2ccc(-n3ccc(Cl)c(Cl)c3=O)cc2)c1C(F)(F)F]

1-(4-(4-acetyl-5-(trifluoromethyl)-1H-pyrazol-1-yl)phenyl)-3,4-dichloropyridin-2(1H)-one (67). 40.5% yield; ¹H NMR (400 MHz, CDCl₃) δ 8.10 (s, 1H), 7.61-7.53 (m, 4H), 7.30 (d, J=7.5 Hz, 1H), 6.47 (d, J=7.5 Hz, 1H), 2.60 (s, 3H).

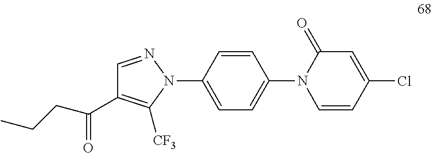 [SMILES: CCCC(=O)c1cnn(-c2ccc(-n3ccc(Cl)cc3=O)cc2)c1C(F)(F)F]

1-(4-(4-butyryl-5-(trifluoromethyl)-1H-pyrazol-1-yl)phenyl)-4-chloropyridin-2(1H)-one (68). 59.0% yield; .y¹H NMR (400 MHz, DMSO-d₆) δ 8.58 (s, 1H), 7.86 (d, J=7.4 Hz, 1H), 7.70 (d, J=8.7 Hz, 2H), 7.64 (d, J=8.7 Hz, 2H), 6.71 (d, J=2.1 Hz, 1H), 6.49 (dd, J₁=7.4 Hz, J₂=2.0 Hz, 1H), 2.95 (t, J=7.1 Hz, 2H), 1.63 (dd, J₁=14.6 Hz, J₂=7.3 Hz, 2H), 0.93 (t, J=7.4 Hz, 3H).

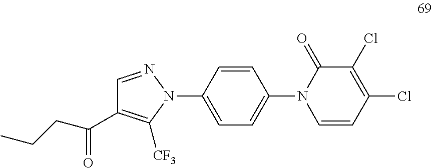 [SMILES: CCCC(=O)c1cnn(-c2ccc(-n3ccc(Cl)c(Cl)c3=O)cc2)c1C(F)(F)F]

1-(4-(4-butyryl-5-(trifluoromethyl)-1H-pyrazol-1-yl)phenyl)-3,4-dichloropyridin-2(1H)-one (69). 46.0% yield; ¹H NMR (400 MHz, DMSO-d₆) δ 8.59 (s, 1H), 7.88 (d, J=7.4 Hz, 1H), 7.70 (q, J=8.8 Hz, 4H), 6.69 (d, J=7.5 Hz, 1H), 2.95 (t, J=7.1 Hz, 2H), 1.63 (dd, J₁=14.5 Hz, J₂=7.3 Hz, 2H), 0.93 (t, J=7.4 Hz, 3H).

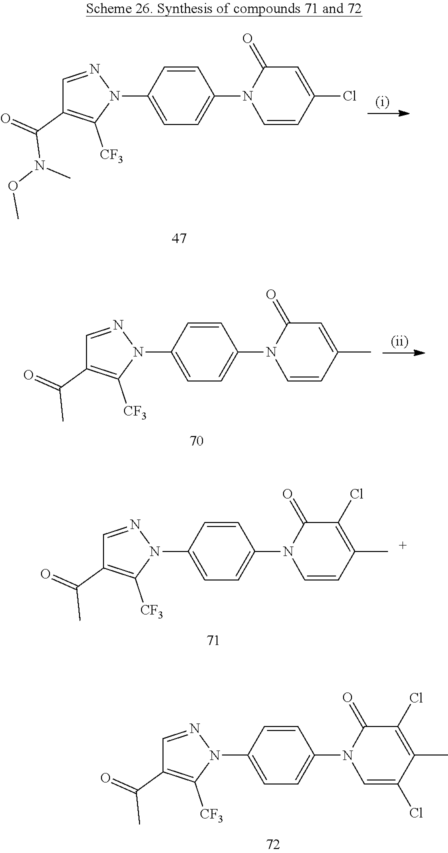

Reagents and conditions: (i) MeMgBr, THF, 0° C.-r.t. (ii) NCS, DMF, 100° C., 8 h.

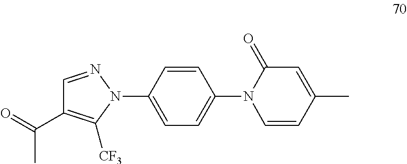 [SMILES: CC(=O)c1cnn(-c2ccc(-n3ccc(C)cc3=O)cc2)c1C(F)(F)F]

1-(4-(4-acetyl-5-(trifluoromethyl)-1H-pyrazol-1-yl)phenyl)-4-methylpyridin-2(1H)-one (70). Compound 47 (854 mg, 2 mmol) was dissolved in 20 mL of anhydrous THF at room temperature under argon. Methylmagnesium bromide (6 mL of 1M THF solution, 6 mmol) was added via a syringe at room temperature under argon. The reaction solution was stirred at room temperature overnight. Then, the reaction was quenched by adding 50 mL of water. The resulted solution was extracted with ethyl acetate (3×30 mL). The organic layer was separated, dried over anhydrous MgSO₄, filtered and concentrated under reduced pressure. The crude solid was purified by flash column chromatography (silica gel, CH₂Cl₂/Acetone=19/1 v/v) to give a white solid (72.7% yield). ¹H NMR (400 MHz, DMSO-d₆) δ 8.57 (s, 1H), 7.68-7.59 (m, 5H), 6.33 (s, 1H), 6.22 (dd, J₁=7.1, J₂=1.7 Hz, 1H), 2.58 (s, 3H), 2.19 (s, 3H). HRMS (ESI): calcd for C₁₈H₁₅F₃N₃O₂ 362.1111 [M+H]⁺, found 362.1122. Purity: 96.0% by HPLC (Rt=3.96 min).

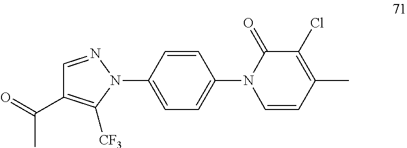 [SMILES: CC(=O)c1cnn(-c2ccc(-n3ccc(C)c(Cl)c3=O)cc2)c1C(F)(F)F]

1-(4-(4-acetyl-5-(trifluoromethyl)-1H-pyrazol-1-yl)phenyl)-3-chloro-4-methylpyridin-2(1H)-one (71). 34.3%; ¹H NMR (400 MHz, CDCl₃) δ 8.09 (s, 1H), 7.57 (s, 4H), 7.25 (d, J=7.1 Hz, 1H), 6.25 (d, J=7.1 Hz, 1H), 2.59 (s, 3H), 2.39 (s, 3H).

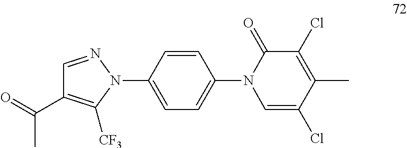 [SMILES: CC(=O)c1cnn(-c2ccc(-n3cc(Cl)c(C)c(Cl)c3=O)cc2)c1C(F)(F)F]

1-(4-(4-acetyl-5-(trifluoromethyl)-1H-pyrazol-1-yl)phenyl)-3,5-dichloro-4-methylpyridin-2(1H)-one (72). The title compound was prepared following the general procedure described above for the chlorination of pyridone ring. 68.2% yield; ¹H NMR (400 MHz, CDCl₃) δ 8.09 (s, 1H), 7.58 (d, J=1.6 Hz, 4H), 7.45 (s, 1H), 2.59 (s, 3H), 2.50 (s, 3H). HRMS (ESI): calcd for C₁₈H₁₂Cl₂F₃N₃NaO₂ 452.0151 [M+Na]⁺, found 452.0136. Purity: 95.6% by HPLC (Rt=4.23 mi).

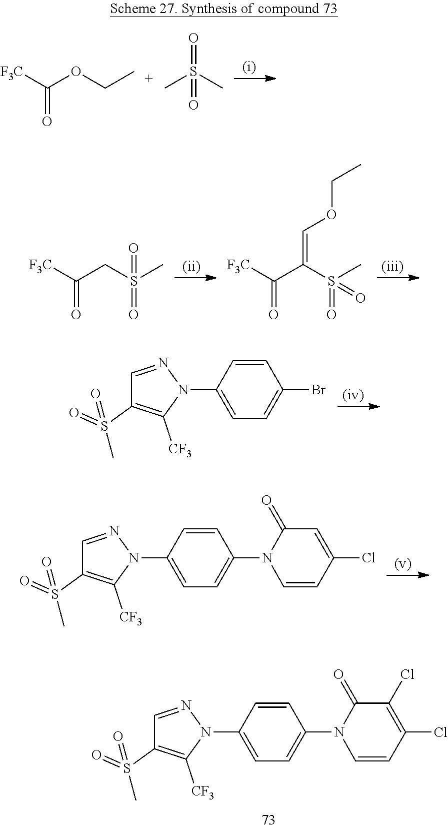

Reagents and conditions: (i) NaH, THF, 60° C., 21 h; (ii) HC(OEt)₃, Ac₂O, reflux, 12 h; (iii) 4-bromophenylhydrazine hydrochloride, EtOH, 60° C., 6 h; (iv) 4-chloropridinone, CuI, DMCDA, K₂CO₃, toluene, reflux, 12 h; (ii) NCS, DMF, rt, overnight.

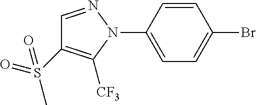 [SMILES: CS(=O)(=O)c1cnn(-c2ccc(Br)cc2)c1C(F)(F)F]

To a solution of dimethylsulfone (5.00 g, 53.1 mmol) in THF (60 mL) was dropwise added a solution of sodium hydride (60% in oil, 3.19 g, 79.7 mmol) in THF (120 mL) over 30 min. The reaction temperature was allowed to increase to 55° C. and stirred for 30 min. After that, ethyl trifluoroacetate (31.7 mL, 266 mmol) was added at 0° C. dropwise over 10 min. Upon completion, the reaction mixture was stirred at 60° C. for 21 h. Then, the reaction mixture was poured into ice water (100 mL) and slowly neutralized with 6N HCl solution. The mixture was extracted with ethyl acetate (100 mL×3). The combined organic layers were dried over Na₂SO₄, and evaporated under reduced pressure to give the crude compound (6.8 g) which was used without further purification in next step.

The crude compound (6.8 g) was dissolved in acetic anhydride (9 mL) and triethyl orthoformate (6.67 g, 45 mmol) was added. The mixture was stirred at reflux for 12 h and the volatiles was removed in vacuo. The residue was redissolved in ethanol (50 mL) and stirred at room temperature overnight. Then, 4-bromophenylhydrazine hydrochloride (4.47 g, 20 mmol) was added and the reaction solution was stirred at 60° C. for 8 h. The solvent was removed in vacuo and saturated NaHCO₃ (100 mL) and ethyl acetate (50 mL) was added to the residue. The organic phase was separated, washed with brine, dried over Na2SO₄, and evaporated under reduced pressure. The residue was purified by flash chromatography to obtain the product as a pale yellow solid (2.07 g, 28% yield). ¹H NMR (400 MHz, CDCl₃) δ 8.17 (s, 1H), 7.69 (d, J=8.8 Hz, 2H), 7.34 (d, J=8.6 Hz, 2H), 3.26 (s, 3H).

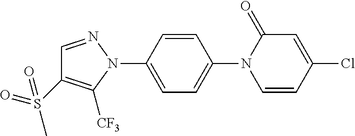 [SMILES: CS(=O)(=O)c1cnn(-c2ccc(-n3ccc(Cl)cc3=O)cc2)c1C(F)(F)F]

4-Chloro-1-(4-(4-(methylsulfonyl)-5-(trifluoromethyl)-1H-pyrazol-1-yl)phenyl)pyridine-2(1H)-one. ¹H NMR (400 MHz, CDCl₃) δ 8.19 (s, 1H), 7.60 (d, J=2.2 Hz, 4H), 7.34 (d, J=7.4 Hz, 1H), 6.75 (d, J=2.2 Hz, 1H), 6.36 (dd, J=7.4, 2.2 Hz, 1H), 3.28 (s, 3H).

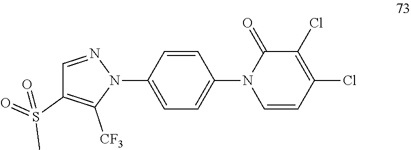

3,4-Dichloro-1-(4-(4-(methylsulfonyl)-5-(trifluoromethyl)-1H-pyrazol-1-yl)phenyl)pyridine-2(1H)-one (73). ¹H NMR (400 MHz, DMSO-d₆) δ 8.40 (s, 1H), 7.89 (d, J=7.5 Hz, 1H), 7.78 (d, J=8.7 Hz, 2H), 7.72 (d, J=8.7 Hz, 2H), 6.70 (d, J=7.5 Hz, 1H), 3.40 (s, 3H).

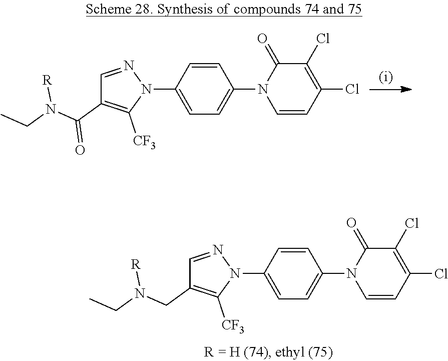

Reagents and conditions: (i) NiCl₂(dme), PhSiH₃, toluene, 120° C., 24 h.

The amide compound (0.20 mmol) and NiCl₂(dme) (4.4 mg, 0.02 mmol) was dissolved in toluene (3 mL) in a 2-dram vial which was charged with a magnetic stir bar. The solvent was flushed with argon for 20 min. PhSiH₃ (98.8 μL, 0.80 mmol) was added under an argon atmosphere and the vial was then quickly sealed with a Teflon-lined screw cap under a flow of argon. The reaction mixture was then allowed to heat to 120° C. and stirred for 24 h. After cooling to room temperature, the reaction mixture was transferred to a separatory funnel with saturated NaHCO₃solution (4 mL) and extracted with EtOAc (3×5 mL). The combined organic layers were dried over anhydrous Na2SO₄. The volatiles were removed under reduced pressure, and the crude residue was purified by flash chromatography (DCM/MeOH=19/1) to afford the desired product as a white solid.

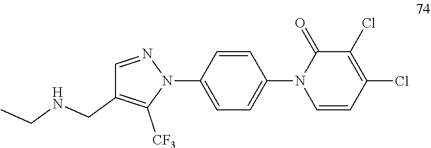 [SMILES: CCNCc1cnn(-c2ccc(-n3ccc(Cl)c(Cl)c3=O)cc2)c1C(F)(F)F]

3,4-dichloro-1-(4-(4-((ethylamino)methyl)-5-(trifluoromethyl)-1H-pyrazol-1-yl)phenyl)pyridin-2(1H)-one (74). 74.4% yield; ¹H NMR (400 MHz, DMSO-d₆) δ 7.91 (s, 1H), 7.87 (d, J=7.4 Hz, 1H), 7.64 (q, J=8.9 Hz, 4H), 6.68 (d, J=7.4 Hz, 1H), 3.73 (s, 2H), 2.57 (q, J=7.1 Hz, 2H), 1.89 (s, 1H), 1.04 (t, J=7.1 Hz, 3H).

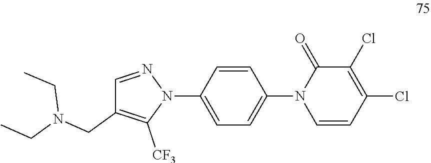 [SMILES: CCN(CC)Cc1cnn(-c2ccc(-n3ccc(Cl)c(Cl)c3=O)cc2)c1C(F)(F)F]

3,4-dichloro-1-(4-(4-((diethylamino)methyl)-5-(trifluoromethyl)-1H-pyrazol-1-yl)phenyl)pyridin-2(1H)-one (75). 39.0% yield; ¹H NMR (400 MHz, DMSO-d₆) δ 7.87 (s, 1H), 7.65 (s, 2H), 6.68 (d, J=6.9 Hz, 1H), 3.58 (s, 2H), 0.99 (t, J=6.8 Hz, 6H).

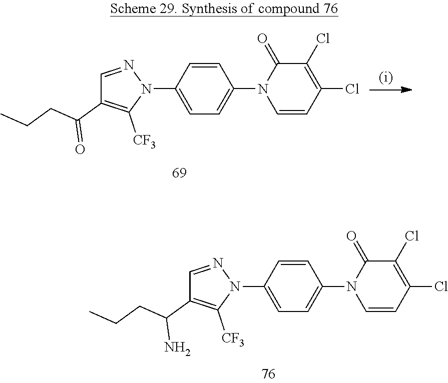 [SMILES: CCCC(=O)c1cnn(-c2ccc(-n3ccc(Cl)c(Cl)c3=O)cc2)c1C(F)(F)F ; CCCC(N)c1cnn(-c2ccc(-n3ccc(Cl)c(Cl)c3=O)cc2)c1C(F)(F)F]

Reagents and conditions: (i) NH₄Ac, NaBH₃CN, t-BuOH, 70° C., overnight.

A solution of compound 69 (89.0 mg, 0.20 mmol) and ammonium acetate (154 mg, 2.0 mmol) in tert-butanol (3 mL) was treated with vigorous stirring. After complete dissolution, NaBH₃CN (62.8 g, 1.0 mmol) were added and the reaction mixture was stirred overnight at 70° C. The solution was concentrated in vacuo and the residue was diluted with EtOAc (10 mL) and washed thoroughly with saturated NaHCO₃solution (20 mL) and saturated brine (20 mL), dried over Na₂SO₄, filtered, and concentrated. The crude residue was purified by flash chromatography (DCM/MeOH=19/1) to afford the desired product.

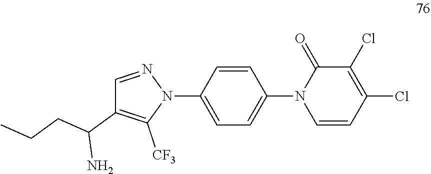 [SMILES: CCCC(N)c1cnn(-c2ccc(-n3ccc(Cl)c(Cl)c3=O)cc2)c1C(F)(F)F]

1-(4-(4-(1-aminobutyl)-5-(trifluoromethyl)-1H-pyrazol-1-yl)phenyl)-3,4-dichloropyridin-2(1H)-one (76). 37.1% yield; ¹H NMR (400 MHz, DMSO-d₆) δ 8.00 (s, 1H), 7.87 (d, J=7.4 Hz, 1H), 7.63 (m, 4H), 6.68 (d, J=7.4 Hz, 1H), 4.01 (s, 1H), 1.90 (s, 2H), 1.60 (s, 2H), 1.40-1.22 (m, 2H), 0.87 (t, J=7.1 Hz, 3H).

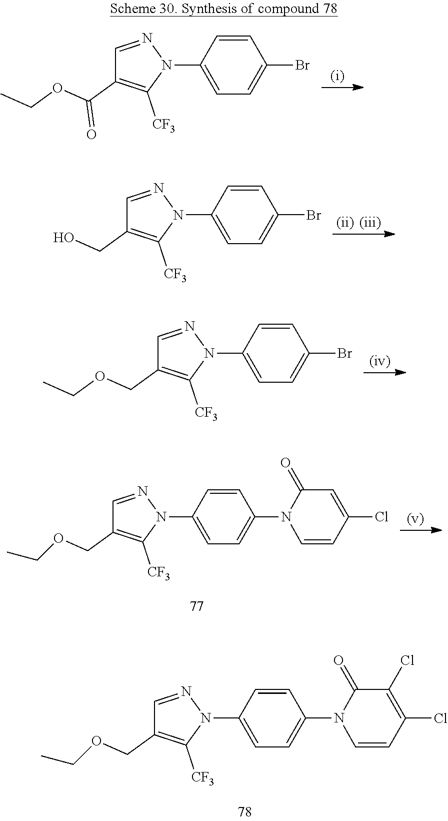 [SMILES: CCOC(=O)c1cnn(-c2ccc(Br)cc2)c1C(F)(F)F ; OCc1cnn(-c2ccc(Br)cc2)c1C(F)(F)F ; CCOCc1cnn(-c2ccc(Br)cc2)c1C(F)(F)F ; CCOCc1cnn(-c2ccc(-n3ccc(Cl)cc3=O)cc2)c1C(F)(F)F ; CCOCc1cnn(-c2ccc(-n3ccc(Cl)c(Cl)c3=O)cc2)c1C(F)(F)F]

Reagents and conditions: (i) NaBH₄, LiBr, EtOH, reflux, 8 h; (ii) NaH, THF, 0° C. to rt, 4 h; (iii) EtI, rt, 3 h; (iv) 4-chloropridinone, CuI, DMCDA, K₂CO₃, toluene, reflux, 12 h; (v) NCS, DMF, 100° C., 8 h.

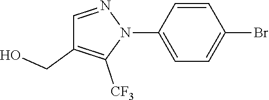 [SMILES: OCc1cnn(-c2ccc(Br)cc2)c1C(F)(F)F]

(1-(4-bromophenyl)-5-(trifluoromethyl)-1H-pyrazol-4-yl)methanol. To a solution of ethyl 1-(4-bromophenyl)-5-(trifluoromethyl)-1H-pyrazole-4-carboxylate (1.82 g, 5 mmol) in ethanol (30 mL) was added sodium borohydride (0.57 g, 15.0 mmol) and lithium bromide (260 mg, 3 mmol). The resulting mixture was stirred at reflux for 8 h. The solvent was removed under reduced pressure. Water (100 mL) was added to the residue and the mixture was extracted with ethyl acetate (3×30 mL). The combined organic layers were dried over Na2SO₄, filtered, and concentrated in vacuo. The crude residue was subjected to flash chromatography method to afford the desired product as an off-white solid (1.0 g, 62.5%). ¹H NMR (400 MHz, CDCl₃) δ 7.79 (s, 1H), 7.62 (d, J=8.7 Hz, 2H), 7.33 (d, J=8.6 Hz, 2H), 4.78 (d, J=0.7 Hz, 2H).

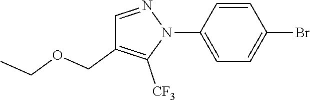 [SMILES: CCOCc1cnn(-c2ccc(Br)cc2)c1C(F)(F)F]

1-(4-bromophenyl)-4-(ethoxymethyl)-5-(trifluoromethyl)-1H-pyrazole. To a solution of SZ-2-163 (963 mg, 3 mmol) in dry THF at 0° C. was added sodium hydride (60% in mineral oil, 181 mg, 4.5 mmol), and the resulting mixture was stirred for 4 h at room temperature. Then, an excess of iodomethane (2.13 g, 15 mmol) was added and the mixture was stirred for 3 h. The solvent was removed under vacuum and the obtained residue was redissolved in dichloromethane (30 mL). The organic phase was washed with water (50 mL×2) and concentrated under vacuum. The crude residue was subjected to flash chromatography method to afford the desired product as an off-white solid (953 mg, 91.0%). ¹H NMR (400 MHz, CDCl₃) δ 7.75 (s, 1H), 7.61 (d, J=8.8 Hz, 2H), 7.32 (d, J=8.6 Hz, 2H), 4.55 (s, 2H), 3.60 (d, J=7.0 Hz, 2H), 1.27 (t, J=7.0 Hz, 3H).

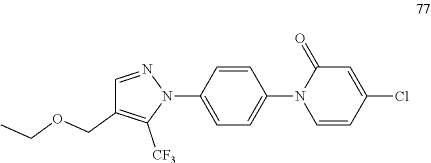 [SMILES: CCOCc1cnn(-c2ccc(-n3ccc(Cl)cc3=O)cc2)c1C(F)(F)F]

4-chloro-1-(4-(4-(ethoxymethyl)-5-(trifluoromethyl)-1H-pyrazol-1-yl)phenyl)pyridin-2(1H)-one (77). 40.8% yield; ¹H NMR (400 MHz, CDCl₃) δ 7.78 (s, 1H), 7.59 (d, J=8.7 Hz, 2H), 7.50 (d, J=8.8 Hz, 2H), 7.33 (d, J=7.4 Hz, 1H), 6.74 (d, J=2.1 Hz, 1H), 6.33 (dd, J₁=7.4 Hz, J₂=2.2 Hz, 1H), 4.57 (s, 2H), 3.61 (q, J=7.0 Hz, 2H), 1.28 (t, J=7.0 Hz, 3H).

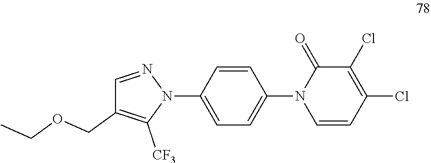 [SMILES: CCOCc1cnn(-c2ccc(-n3ccc(Cl)c(Cl)c3=O)cc2)c1C(F)(F)F]

3,4-dichloro-1-(4-(4-(ethoxymethyl)-5-(trifluoromethyl)-1H-pyrazol-1-yl)phenyl)pyridin-2(1H)-one (78). 35.0% yield; ¹H NMR (400 MHz, DMSO-d₆) δ 7.95 (s, 1H), 7.87 (d, J=7.5 Hz, 1H), 7.67 (s, 4H), 6.68 (d, J=7.5 Hz, 1H), 4.51 (s, 2H), 3.52 (q, J=7.0 Hz, 2H), 1.15 (t, J=7.0 Hz, 3H).

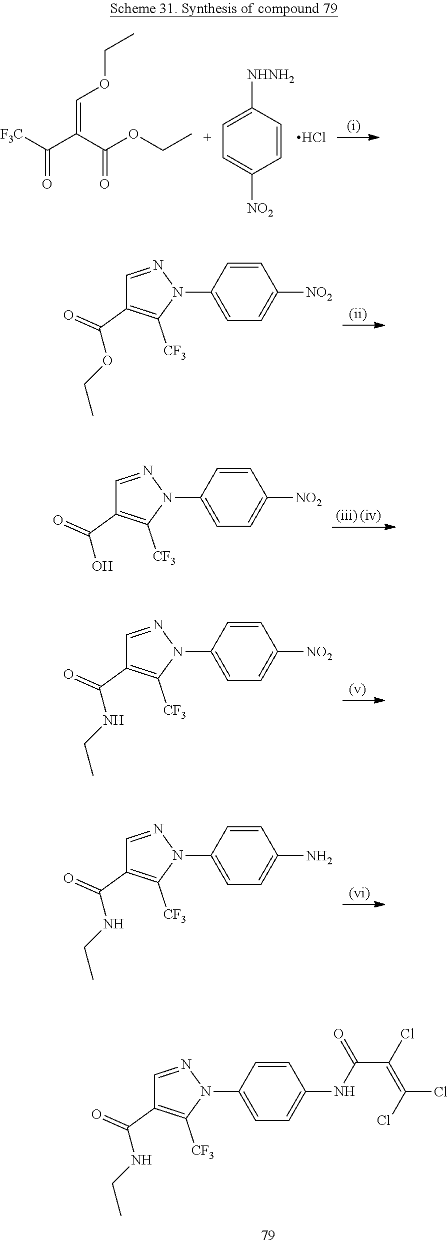

Reagents and conditions: (i) EtOH, 60° C., 6 h; (ii) 1 M KOH (aq.), EtOH, H₂O, rt, 8 h; (iii) SOCl₂, CH₂Cl₂, reflux for 4 h; (iv) ethylamine, TEA, THF, r.t., 12 h; (v) H₂, Pd/C, MeOH, r.t., 8 h; (vi) 2,3,3-trichloroacryloyl chloride, THF, TEA, r.t., 12 h.

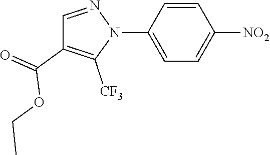 [SMILES: CCOC(=O)c1cnn(-c2ccc([N+](=O)[O-])cc2)c1C(F)(F)F]

Ethyl 1-(4-nitrophenyl)-5-(trifluoromethyl)-1H-pyrazole-4-carboxylate. 82.1% yield; ¹H NMR (400 MHz, CDCl₃): δ 8.38 (d, J=8.8 Hz, 2H), 8.18 (s, 1H), 7.66 (d, J=8.8 Hz, 2H), 4.40 (q, J=7.2 Hz, 2H), 1.40 (t, J=7.2 Hz, 3H).

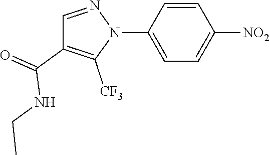 [SMILES: CCNC(=O)c1cnn(-c2ccc([N+](=O)[O-])cc2)c1C(F)(F)F]

N-ethyl-1-(4-nitrophenyl)-5-(trifluoromethyl)-1H-pyrazole-4-carboxamide. 86.9% yield; ¹H NMR (400 MHz, DMSO-d₆): δ 8.62 (t, J=5.2 Hz, 1H), 8.43 (d, J=7.2 Hz, 2H), 8.24 (s, 1H), 7.84 (d, J=8.8 Hz, 2H), 3.28-3.25 (m, 2H), 1.12 (t, J=7.2 Hz, 3H).

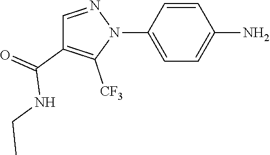 [SMILES: CCNC(=O)c1cnn(-c2ccc(N)cc2)c1C(F)(F)F]

1-(4-Aminophenyl)-N-ethyl-5-(trifluoromethyl)-1H-pyrazole-4-carboxamide. Compound N-ethyl-1-(4-nitrophenyl)-5-(trifluoromethyl)-1H-pyrazole-4-carboxamide (1.00 g, 3.05 mmol) was dissolved in 50 mL of methanol and 10 mL of ethyl acetate at room temperature. To this solution was added Pd/C (0.10 g, 10% Pd base). The reaction vessel was vacuumed, and hydrogen gas was introduced by a hydrogen balloon at room temperature. The reaction mixture was stirred at room temperature under hydrogen atmosphere for 12 hours and filtered through a celite. The solution was evaporated to dryness. The crude was subjected to flash column chromatography (silica gel, CH₂Cl₂/MeOH=19/1 v/v) to afford the desired amino compound as a white solid product (0.80 g, 88.0%). ¹H NMR (400 MHz, DMSO-d₆): 8.48 (t, J=5.6 Hz, 1H), 7.97 (s, 1H), 7.06 (d, J=8.8 Hz, 2H), 6.62 (d, J=8.8 Hz, 2H), 5.59 (s, 2H), 3.26-3.19 (m, 2H), 1.09 (t, J=7.2 Hz, 3H).

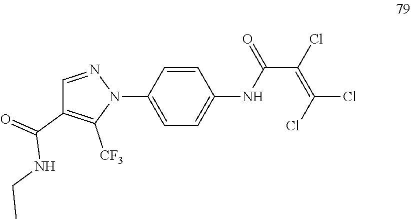 [SMILES: CCNC(=O)c1cnn(-c2ccc(NC(=O)C(Cl)=C(Cl)Cl)cc2)c1C(F)(F)F]

N-ethyl-1-(4-(2,3,3-trichloroacrylamido)phenyl)-5-(trifluoromethyl)-1H-pyrazole-4-carboxamide (79). 2,3,3-trichloroacrylic acid (0.44 g, 2.561 mmol) was dissolved in 10 mL of anhydrous methylene chloride at room temperature under nitrogen. Thionyl chloride (0.90 g, 7.53 mmol) and three drops of DMF were added to the above solution via a syringe at room temperature. The reaction mixture was stirred and heated to reflux for 4 h. Then, the volatile was removed under reduced pressure to give the corresponding acyl chloride as a yellow oil which was dissolved in 10 mL of anhydrous THF. This acyl chloride THF solution was added to the THF (20 mL) solution of compound 1-(4-Aminophenyl)-N-ethyl-5-(trifluoromethyl)-1H-pyrazole-4-carboxamide (0.75 g, 2.51 mmol) at room temperature. The reaction mixture was stirred at room temperature overnight. The solvent was removed under reduced pressure and the residue was subjected to flash column chromatography (silica gel, CH₂Cl₂/MeOH=9/1 v/v) to afford the desired product as a white solid (0.95 g, 82.2%). ¹H NMR (400 MHz, CDCl₃): δ 11.33 (s, 1H), 8.56 (t, J=5.2 Hz, 1H), 8.10 (s, 1H), 7.80 (d, J=8.4 Hz, 2H), 7.53 (d, J=8.4 Hz, 2H), 3.26-3.23 (m, 2H), 1.11 (t, J=7.2 Hz, 3H). HRMS (ESI): calcd for C₁₆H₁₃Cl₃F₃N₄O₂ 455.0051 [M+H]⁺, found 455.0107. Purity: 96.4% by HPLC (Rt=3.74 min).

### Example 2

### Biological Activity of Synthesized Compounds

### Cell Culture and Electrophysiology

Human embryonic kidney 293 (HEK293; catalog number CRL-1573) cells were cultured in Dulbecco's modified Eagle's medium (Invitrogen) supplemented with 10% fetal bovine serum and 1% penicillin-streptomycin at 37° C. and 5% CO2. Constructs cloned in pMO (a pcDNA3-modified vector) were co-transfected with GFP-pMO, using Lipofectamine 2000 (Invitrogen) according to the manufacturer's instructions. Macroscopic currents in the whole-cell patch clamp configuration were recorded 18-24 h post-transfection. The extracellular solution contained 140 mM NaCl, 6 mM KCl, 2 mM MgCl2, 10 mM HEPES, and 10 mM glucose (pH 7.4). Pipettes were made of borosilicate glass (outer diameter, 1.5 mm; inner diameter, 1.10 mm; Sutter Instruments) and fire-polished with a resistance between 2.8 and 4.0 megaohms when filled with an intracellular solution that contained 140 mM CsCl, 5 mM EGTA, and 10 mM HEPES, pH 7.2. Currents were recorded with a Multiclamp 700B amplifier (Molecular Devices) using a 1-s ramp from −100 to 100 mV. Agonist and antagonists perfused were dissolved in the bath solution prior experiments: TRP channel agonists Allyl isothiocyanate (AITC; TRPA1), capsaicin (TRPV1), and menthol (TRPM8) were obtained from Sigma, GSK1016790A (TRPV4) from Cayman chemicals, and GSK1702934A (TRPC3, TRPC6 and TRPC7) from Tocris Bioscience. Antagonist Pyr3 was obtained from Tocris Bioscience, all other antagonists were synthesized in house. Data were acquired with a sampling rate of 20 kHz, low-pass filtered (10 kHz), and analyzed offline using Clampfit v10.4.2.0 (Molecular Devices).

### Data Analysis

Electrophysiology results were expressed as means±SD. Data were plotted, and sigmoidal fitting was done using OriginPro (from OriginLab), with the following Boltzmann function:

$${f}_{( x )} = {A}_{2} + \frac{{A}_{1} - {A}_{2}}{1 + {e}^{( ( X - {X}_{0} / dX )}} , where \qquad ( 1 )$$

$${A}_{2} = final value , {A}_{1} = initial value ; \\ {X}_{0} = center , and dX = time constant .$$

### In Vivo Toxicity Study

The in vivo study was performed in compliance with the NIH Animal Use Guidelines and protocol approved by the Institutional Animal Care and Use Committee (IACUC) at the University of Tennessee Health Science Center (UTHSC, Memphis, Tenn.). NSG mice at 8 weeks (n=2) and ICR mice at 12 weeks (n=3) were housed with a 12:12-hour light-dark cycle and fed a breeder chow diet. Daily IP injections of 200 mg/kg of compounds 22 or 79 were administered for five consecutive injections in five mice, respectively, which were followed to monitor the mortality rate. Body weights of the mice were recorded daily.

### TRPC3 as a Drug Target for Epilepsy

Transient receptor potential canonical 3 (TRPC3) is a member of the TRP family channels that control the influxes of Ca2+ and other cations. TRPC3 is abundantly expressed in the neocortex and hippocampus, where it co-localizes with the brain-derived neurotrophic factor (BDNF) and tropomyosin-related kinase receptor B (TrkB), regulating BDNF/TrkB-mediated dendritic remodeling in pyramidal neurons. Accumulating reports suggest that excessive BDNF/TrkB signaling activity contributes to spontaneous recurrent seizures (SRSs) after status epilepticus (SE), indicating a significant role of TRPC3 in epileptic seizures. Consistent with this, TRPC3 knockdown markedly reduces the duration and severity of pilocarpine-induced seizures in mice, suggesting that TRPC3 inhibition is a novel anti-seizure and/or anti-epileptogenic strategy.

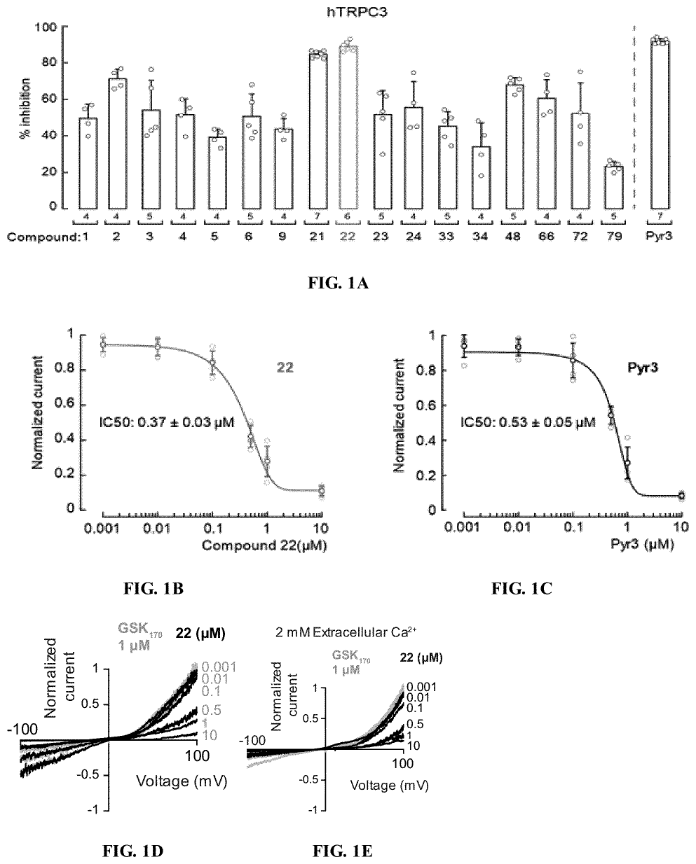

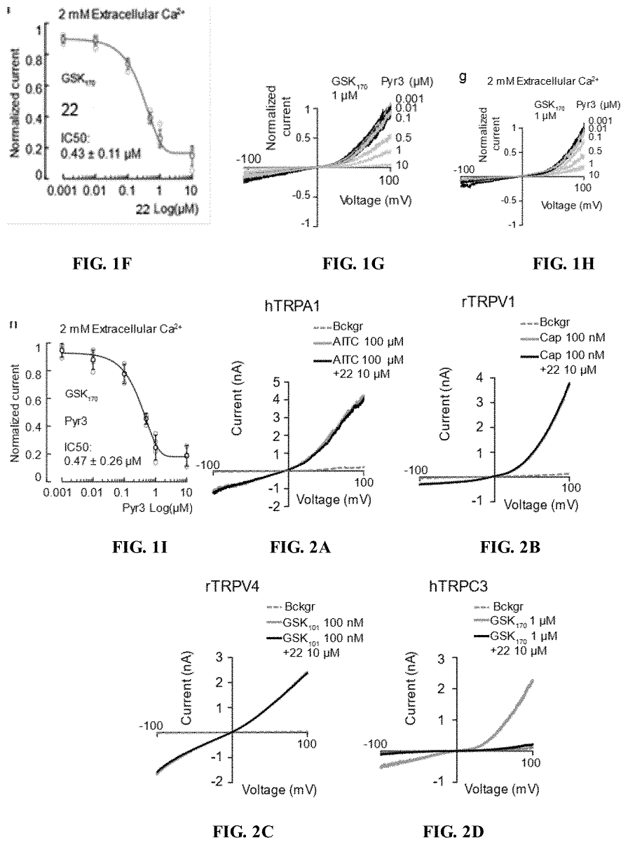

Using the patch clamp technique in the whole-cell configuration, compounds were screened by challenging hTRPC3 overexpressing HEK293 cells with GSK1702934A to activate TRPC3 and each of the synthesized compounds (10 μM) individually in the presence of the agonist. Compound 22 was determined as the currently most potent compound in hand and was used in further biology studies (FIG. 1A).

To avoid tachyphylaxis and determine the IC50 of compound 22, each point of the dose response curve (FIG. 1B) was done individually on a cell and exchanging coverslips between cells. Using this approach, the IC50 of compound 22 was determined (0.37±0.03 μM; SEM) to be lower than that of Pyr3 (0.53±0.05 μM, SEM; FIG. 1C), supporting the idea use of compound 22 as a potent inhibitor of TRPC3. FIGS. 1D-1I show the inhibition of TRPC3-mediated currents by compound 22 and Pyr3 (serving as the reference control). The data shows that compound 22 is more potent than Pyr3 in both assays. Similar results were obtained when OAG was used as the agonist (data not shown).

Compound 22 selectively inhibits TRPC3 among closely related TRP channels.

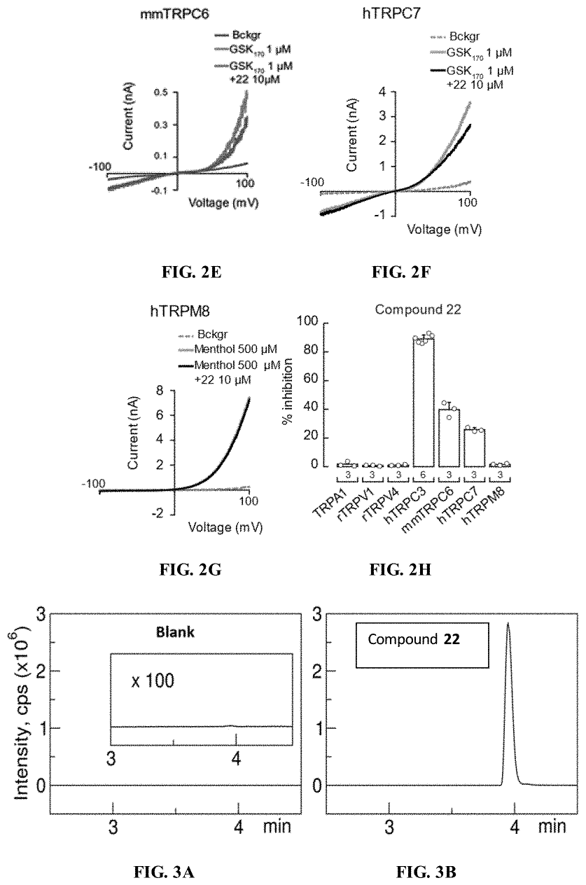

The selectivity of this compound across members of the TRP superfamily of ion channels was subsequently tested. The patch clamp technique was used to record whole-cell currents of HEK 293 cells overexpressing hTRPA1, rTRPV1, rTRPV4, hTRPC3, mmTRPC6, hTRPC7, and hTRPM8 (FIGS. 2A-2G). The experiments were performed using solutions without Ca2+ to avoid TRP channel desensitization. After fully activating these channels, compound 22 (10 μM) was perfused with each of their respective agonists. At this saturating concentration of 10 μM, compound 22 inhibited <5% of the current of hTRPA1, rTRPV1, rTRPV4, and hTRPM8 (FIG. 2H); and around 39% of hTRPC6 and 22% of hTRPC7 (FIG. 2H), which are closely related to TRPC3. Compound 22 does not inhibit TRPV1, TRPV4, TRPM8, and TRPA1. Compound 22 was subsequently tested against TRPC6 and TRPC7, which are closely related to TRPC3. Compound 22 inhibits TRPC6 and TRPC7 on a much weaker basis than TRPC3 (FIG. 2H). The data demonstrate that compound 22 is highly selective for TRPC3. Taken together, the results demonstrate that compound 22 is a potent and highly selective inhibitor of hTRPC3. Activities of additional selected compounds are listed in Table 4.

TABLE 4
Activities of exemplary compounds

| | hTRPC3 % Inhibition | hTRPC3 IC₅₀ (μM, |
|---|---|---|
| Compound No. | at 10 μM (mean ± SEM) | mean ± SEM) |
| 1 | 49.48 ± 3.91 | |
| 2 | 71.12 ± 2.66 | |
| 3 | 54.03 ± 7.25 | |
| 4 | 51.43 ± 4.35 | |
| 5 | 39.1 ± 2.31 | |
| 6 | 50.51 ± 5.49 | |
| 9 | 39.10 ± 2.31 | |
| 21 | 84.52 ± 0.722 | |
| 22 | 89.03 ± 1.14 | 0.37 ± 0.03 |
| 23 | 51.52 ± 6 | |
| 24 | 55.52 ± 7.07 | |
| 25 | 76.04 ± 6.65 | |
| 27 | 89.37 ± 6.45 | |
| 32 | 95.95 ± 1.37 | 0.35 ± 0.09 |
| 33 | 45.21 ± 3.59 | |
| 34 | 34 ± 6.52 | |
| 37 | 40.23 ± 10.27 | |
| 38 | 86.20 ± 3.96 | |
| 39 | 71.54 ± 4.51 | |
| 40 | 40.23 ± 3.95 | |
| 41 | 25.81 ± 8.57 | |
| 43 | 94.84 ± 2.75 | 4.32 ± 0.98 |
| 44 | 74.82 ± 5.65 | |
| 45 | 86.65 ± 3.82 | |
| 46 | 92.86 ± 6.78 | 0.29 ± 0.05 |
| 48 | 67.81 ± 1.75 | |
| 49 | 97.23 ± 0.89 | 1.10 ± 0.24 |
| 50 | 98.08 ± 0.85 | 2.56 ± 0.52 |
| 51 | 72.34 ± 2.88 | |
| 52 | 88.78 ± 6.37 | |
| 54 | 77.68 ± 2.71 | |
| 58 | 85.95 ± 2.80 | |
| 59 | 95.87 ± 3.72 | 0.87 ± 0.17 |
| 61 | 47.09 ± 8.29 | |
| 63 | 86.91 ± 3.83 | |
| 65 | 54.93 ± 4.61 | |
| 66 | 60.43 ± 5.12 | |
| 67 | 88.73 ± 2.83 | |
| 69 | 63.41 ± 4.83 | |
| 71 | 57.77 ± 8.74 | |
| 72 | 52.13 ± 8.41 | |
| 73 | 72.71 ± 7.96 | |
| 74 | 97.96 ± 0.83 | 0.09 ± 0.02 |
| 76 | 32.97 ± 5.58 | |
| 78 | 68.34 ± 2.11 | |
| 79 | 23.21 ± 1.23 | |
| Pyr3 | 91.72 ± 0.53 | 0.53 ± 0.05 |

### Compound 22 Directly Binds to TRPC3

LC-MS technique was employed to find some evidence of the on-target engagement of compound 22 with TRPC3. Biochemical quantities of affinity-purified TRPC3 protein was successfully generated before and was confirmed by SDS-PAGE.21 After incubation of compound 22 with TRPC3 protein for 1 h, repeated size-exclusion chromatography was used to completely remove free compound 22 from the protein-bound fraction. Methanol was then added to this complex to denature the protein and extract the bound compound. As shown in FIGS. 3A and 3B, the presence of compound 22 in the extract solution was detected by LC-MS while no peaks are shown in the controls. Since any unbound compound was washed out during the complex preparation, a conclusion of direct binding of compound 22 to TRPC3 can be drawn from these results.

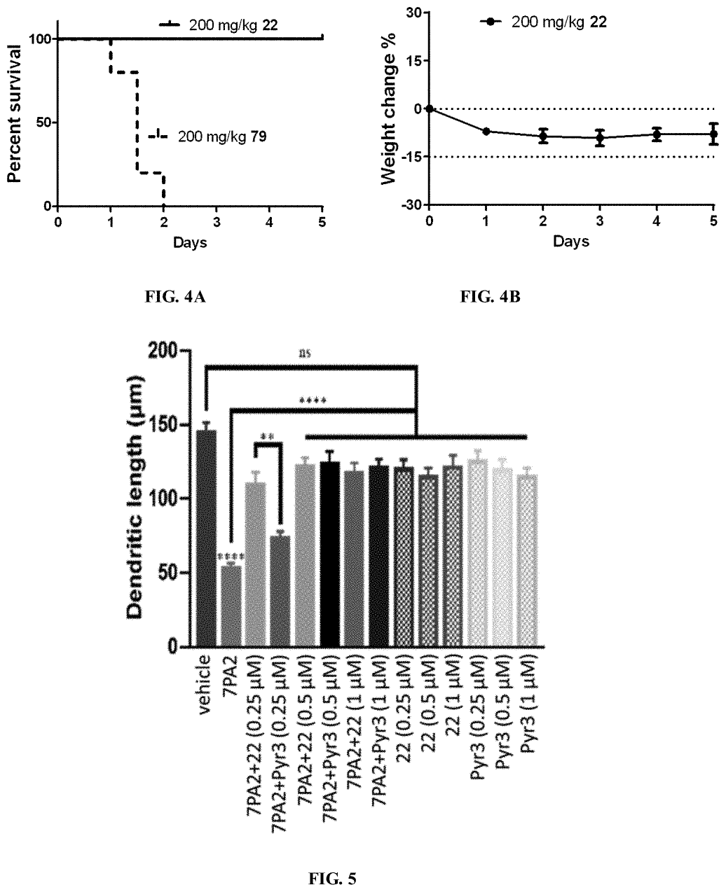

The metabolic stability of compound 22 was tested prior to the next phase of in vivo study. As expected, the half-life time was extended dramatically from less than 15 minutes for Pyr3 to more than 4 hours for compound 22 in mouse, rat or human liver microsomes, in line with our design and hypothesis that replacement of the ester by amide group can overcome structural liabilities of Pyr3. The maximum tolerated dose (MTD) study was subsequently performed to investigate its in vivo toxicity (FIGS. 4A and 4B). The second major limitation of Pyr3 is its toxicity due to its alkylating tail moiety. While replacing only the fast-metabolizing head ester group in Pyr3 with a stable amide (compound 79) improved metabolic stability, a single IP injection of compound 79 at 200 mg/kg killed all mice within 24 h. In contrast, after daily IP injections of compound 22 at 200 mg/kg/day for five consecutive days, no acute toxicities or body weight loss was observed, confirming the design of compound 22 to “dial-out” the toxic alkylating moiety in Pyr3. Compound 22 has significantly improved safety profiles as shown by survivals.

Because Pyr3 undergoes fast hydrolysis to its inactive metabolite Pyr8 and they both showed rapid blood clearance in mice,²⁰ compound 22 bearing the same trichloroacrylic amide group as Pyr3 was incorporated as the comparison treatment. Compound 79 is a metabolically stable version of Pyr3. Mice treated daily with compound 22 or compound 79 (n=5) for 5 consecutive days. All the mice were treated with either compound 22 or 79 at 200 mg/kg (IP). To our surprise, all the compound 79-treated mice were found dead within 48 h, while all the 8 treated mice were alive and no significant body weight loss was observed. Since the only difference between compounds 22 and 79 in structure is their tail moiety, this study provides firm evidence that our rational structural optimization can dramatically decrease the toxicity cause by the trichloroacrylic amide group, which is consistent with our design.

This work demonstrates that new compounds, such as compound 22, which overcame two main limitations of Pyr3, were designed and synthesized. The bioisosteric replacement of the ester by an amide group dramatically enhances the metabolic stability, and cyclization of the trichloroacrylic amide group to form pyridone ring significantly improves the safety profile of the original Pyr3 scaffold. For example, compound 22 combines high potency and reasonable selectivity for TRPC3 versus other TRP subfamilies. Collectively these findings show that the properties of these compounds, in particular compound 22, can lead to a safer and more efficient therapeutic approach to TRPC3-related diseases.

To explore the potential biological effects of compound 22 in a TRPC3 related disease model, its neuroprotective ability in primary cultured neurons (14 DIV prepared from E17 rats as described) was evaluated. The effects on neuron dendritic morphology were measured by imaging. Mature hippocampal neurons (DIV=14) were treated with CHO control medium, or naturally secreted Aβ-containing conditioned medium (7PA2), or in combination with compound 22 or Pyr3 at different concentrations, for 16 hours, followed by immunocytochemistry using an antibody against MAP2 and DAPI (data not shown). The results demonstrate that compound 22 and Pyr3 protect against Aβ-induced damage to dendrites. Hippocampal neurons (DIV=14) were treated with compound 22 or Pyr3 alone at different concentrations, followed by immunocytochemistry staining with MAP2 and DAPI (data not shown). The results show a lack of toxicity of the inhibitor compounds in neuronal culture. Compound 22 displayed similar protective effects as Pyr3 in preventing soluble oligomeric Aβ-induced synaptic toxicity on dendritic spine morphology as evidenced by MAP-2 staining (FIG. 5).

### Brain Permeability and Half-Life of Compound 22

The brain permeability of compound 22 was evaluated with systemic administration (20 mg/kg, i.v.) in C57BL/6 mice. Blood samples were collected at nine time points after compound administration: 5 min, 15 min, 30 min, 1 hr, 2 hr, 4 hr, 8 hr, 16 hr, and 24 hr. Brain homogenates were then extracted and analyzed for concentrations of compound 22 by liquid chromatography-mass spectrometry (LC-MS). The half-life (T½) in the brain was calculated by GraphPad Prism software.

As a key member of transient receptor potential (TRP) superfamily, TRP canonical 3 (TRPC3) regulates calcium homeostasis via engaging both store-operated Ca2+ entry and receptor-operated Ca2+ influx and is thought to essentially contribute to neuronal excitability. Genetic ablation of TRPC3 caused a marked reduction in pilocarpine-induced seizures in mice, suggesting that TRPC3 inhibition might represent a novel anti-seizure strategy. To test whether this scaffold has efficacy in seizures, experiments were conducted to determine its brian permeability and half-life. Compound 22 has a plasma T½˜4 hr and brain-to-plasma ratio ˜0.15 after systemic administration in mice. The brain concentration of compound 22 is well above its TRPC3 IC50 for 18-20 hrs, suggesting a sufficient duration of drug exposure in the brain following one-dose injection and this scaffold is friendly for brain penetration. The data showed that compound 22 has favorable brain permeability and half-life in mice (FIG. 6), justifying its uses for CNS conditions.

Compound 22 shows efficacy in two clinically relevant epilepsy mouse models: the pilocarpine model of SE and the pentylenetetrazole model of SE.

Mouse Pilocarpine Model of SE. Mice were first injected with methylscopolamine and terbutaline (2 mg/kg each in saline, i.p.) to minimize the unwanted effects of pilocarpine in the periphery. Thirty minutes later, pilocarpine (250 mg/kg in saline, freshly prepared, i.p.) was injected to induce seizures in mice. To determine the anti-seizure effects of compound 22, compound 22 was administered either before pilocarpine injection or after the behavioral seizures were well established. Control mice received methylscopolamine and terbutaline, followed by saline injection instead of pilocarpine. Behavioral seizures were classified using a modified Racine scale as we previously described (Table 5).²²⁻²⁵ SE was defined by non-intermittent seizure activity (stage 5), which was indicated by continuous generalized clonic seizures without returning to lower-stage seizures (Table 5).

TABLE 5
Modified Racine scale for scoring the convulsive seizures
after systemic administration of pilocarpine in mice.

| Seizure score | Observed motor behavior |
|---|---|
| 0 | Normal behavior - walking, exploring, sniffing, and |
| | grooming |
| 1 | Immobile, staring, jumpy, and curled-up posture |
| 2 | Automatisms - repetitive blinking, chewing, head |
| | bobbing, vibrissae twitching, scratching, face- |
| | washing, and “star-gazing” |
| 3 | Partial body clonus, occasional myoclonic jerks, and |
| | shivering |
| 4 | Whole body clonus, “corkscrew” turning & flipping, |
| | loss of posture, rearing and falling |
| 5 | SE onset: non-intermittent seizure activity |
| 6 | Wild running, bouncing, and tonic seizures |
| 7 | Death |

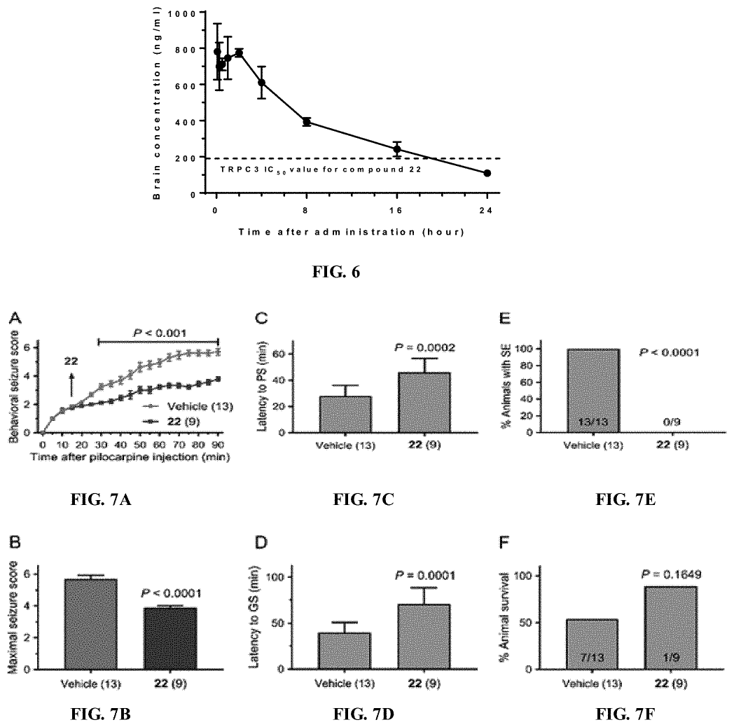

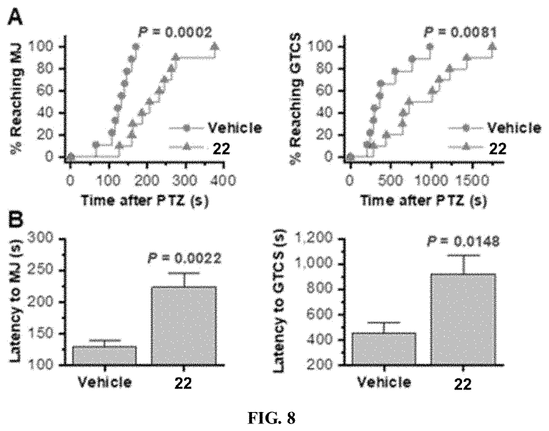

Systemic treatment with compound 22 before pilocarpine injection in mice significantly impaired the initiation of behavioral seizures. This anti-seizure effect was fully recapitulated when compound 22 was administered after pilocarpine-induced behavioral seizures were established, suggesting that TRPC3 inhibition mitigated the progression of chemoconvulsant seizures (FIGS. 7A-7F). Moreover, treatment with compound 22 diminished electrographic seizures after pilocarpine injection, detected by electroencephalography monitoring. A single dose of compound 22 (100 mg/kg, i.p) after the onset of pilocarpine-induced seizures (stage 2) in mice decreased the overall seizure intensity (FIG. 7A) as well as the maximal seizure scores during a 90-minute observation (FIG. 7B). These results show that pharmacological inhibition of the TRPC3 channels by the novel compound 22 represents a new anti-seizure strategy engaging a novel mechanism of action.

### Mouse Pentylenetetrazole (PTZ) Model of SE

To ensure that the efficacy observed in FIG. 7 is not associated with this particular mouse model of epilepsy, we examined the effects of our current lead compound 22 on pentylenetetrazole (PTZ)-induced seizures in mice. As a GABA receptor antagonist, PTZ is another convulsant chemical commonly used to generate seizures in experimental rodents. Young adult C57BL/6 mice (male, 8-9 weeks) were first treated by 22 (100 mg/kg, i.p.) or vehicle. Thirty minutes later, mice were subcutaneously injected with (PTZ, ˜80 mg/kg) to induced seizures. Animals were placed in a plexiglass chamber, and the latencies to the first myoclonic jerk (MJ) and the first generalized tonic-clonic seizure (GTCS) were recorded during a 30-min period of observation. We found that the treatment with compound 22 substantially reduced the percentages of mice that reached MJ (p=0.0002) and GTCS (p=0.0081) when compared to treatment with vehicle only (FIG. 8A). In line, 22-treated mice showed significantly increased latencies to their first MJ (p=0.0022) and GTCS (p=0.0148) compared to the vehicle-treated cohorts (FIG. 8B). These results demonstrated powerful anti-seizure effects of our lead compound 22 in mouse PTZ model. Given that PTZ (FIG. 8) and pilocarpine (FIG. 7) trigger seizures via different mechanisms of actions, it is unlikely the anti-seizure effects of our lead compound 22 are model-specific. Collectively, these results show that pharmacological inhibition of the TRPC3 channels by the novel compound 22 represents a new anti-seizure strategy engaging a novel mechanism of action.

### REFERENCES

1. Venkatachalam, et al. Annu Rev Biochem 2007, 76, 387-417.
2. Mori, et al. J Exp Med 2002, 195 (6), 673-681.
3. Huang, et al. J Am Soc Nephrol 2004, 15 (7), 1690-1699.
4. Numaga-Tomita, et al. Pflug Arch Eur J Phy 2019, 471 (3), 507-517.
5. Hao, et al. Sci China Life Sci 2018, 61 (3), 253-265.
6. Li, et al. Neuron 1999, 24 (1), 261-273.
7. Riccio, et al. Mol Brain Res 2002, 109 (1-2), 95-104.
8. Li, et al. J Neurophysiol 2010, 103 (5), 2846-2856.
9. Amaral, et al. J Neurosci 2007, 27 (19), 5179-5189.
10. Bathina, et al. Arch Med Sci 2015, 11 (6), 1164-1178.
11. Bush, et al. J Biol Chem 2006, 281 (44), 33487-33496.
12. Cohen, et al. J Immunol 2009, 183 (10), 6478-6488.
13. Philipp, et al. J Biol Chem 2003, 278 (29), 26629-26638.
14. Oda, et al. J Physiol Sci 2017, 67 (4), 497-505.
15. Wang, et al. Cancers 2019, 11 (4), 558-573.
16. Aydar, et al. Cancer Cell Int 2009, 9, 23-34.
17. Koenig, et al. J Pharmacol Exp Ther 2013, 344 (1), 33-40.
18. Zhu, et al. J Biol Chem 1998, 273 (1), 133-142.
19. Kamouchi, et al. J Physiol-London 1999, 518 (2), 345-358.
20. Kiyonaka, et al. P Natl Acad Sci USA 2009, 106 (13), 5400-5405.
21. Hagimori, et al. Medchemcomm 2016, 7 (5), 1003-1006.

1. A compound having Formula (IV) or (V): 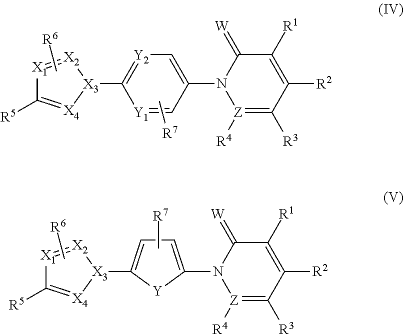
or pharmaceutically acceptable salts and prodrugs thereof wherein,
W is absent, O, S or NR; X₁, X₂, X₃, and X₄ are independently C, O, S, or NR; Y, Y₁, and Y₂ are independently C, O, S or N; Z is C or N; R is H or alkyl; R¹, R², R³, and R⁴ are independently H, hydroxyl, halogens, alkyl, or alkoxy; R⁵ is H, halogens, alkyl, —CH₂N(R⁸)₂, —CH₂OR⁸, —COR⁸, —COOR⁸, —CON(R⁸)₂, —SO₂R⁸, —NHCOR⁸, aryl, or heterocyclic ring; R⁶ and R⁸ are independently H, NHR, OR, alkyl, aryl, or hetero-aryl; and R⁷ is H, halogens, alkyl, or alkoxy.

2. The compound of claim 1, wherein the compound has a structure of Formula (VI). 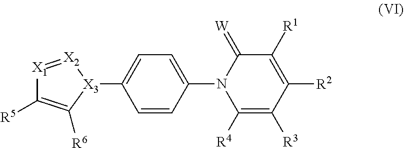
or pharmaceutically acceptable salts and prodrugs thereof wherein,
W is O, S, or NR; X₁, X₂, and X₃ are independently C, O, S, or NR; R is H or alkyl; R¹, R², R³ and R⁴ are independently H, hydroxyl, halogens, alkyl, or alkoxy; R⁵ is H, halogens, alkyl, —CH₂N(R⁸)₂, —CH₂OR⁸, —COR⁸, —COOR⁸, —CON(R⁸)₂, —SO₂R⁸, —NHCOR⁸, aryl, or a heterocyclic ring; R⁶ and R⁸ are independently H, NHR, OR, alkyl, aryl, or hetero-aryl.

3. The compound of claim 1, wherein the compound is a salt or prodrug of Formula (IV) or (V).

4. The compound of claim 3, wherein W is O or NR; R is H or alkyl.

5. The compound of claim 3, wherein R¹, R², R³ and R⁴ are independently H, hydroxyl, or halogens.

6. The compound of claim 3, wherein X₁, X₂, and X₃ are independently C, O, or NR.

7. The compound of claim 3, wherein R⁵ is —CH₂N(R⁸)₂, —CH₂OR⁸, —COR⁸, —COOR⁸, —CON(R⁸)₂, —SO₂R⁸, —NHCOR⁸, aryl, or heterocyclic ring; R⁸ is H, NHR, OR, alkyl, aryl, or hetero-aryl.

8. The compound of claim 7, wherein R⁵ is —CH₂N(R⁸)₂, —CON(R⁸)₂, or heterocyclic ring; R⁸ is H or alkyl.

9. The compound of claim 8, wherein R⁶ is trihaloalkyl.

10. A pharmaceutical composition comprising the compound according to claim 9 and a pharmaceutically acceptable carrier.

11. A method of treating hypertension, cancer, and/or Alzheimer's disease or treating or ameliorating one or more symptoms associated with hypertension, cancer, and/or Alzheimer's disease in a subject in need thereof comprising
(i) administering to the subject the pharmaceutical composition of claim 10,
wherein the compound is in a therapeutically effective amount to treat hypertension, cancer, and/or Alzheimer's disease, or treat or ameliorate one more symptoms associated with hypertension, cancer, and/or Alzheimer's disease.

12. A method of inhibiting TRPC3 in a subject in need thereof, comprising
(i) administering to the subject the pharmaceutical composition of claim 10,
wherein the compound is in a therapeutically effective amount to inhibit the protein in the subject.

13. (canceled)

14. A method of treating one or more symptoms associated with epilepsy in a subject in need thereof comprising
(i) administering to the subject the pharmaceutical composition of claim 10,
wherein the compound is in a therapeutically effective amount to treat epilepsy or ameliorate one more symptoms associated with epilepsy.

15. The method of claim 14, wherein the compound effectively inhibits TRPC3 in the subject.

16. The method of claim 14, wherein the compound does not inhibit TRPV1, TRPV4, TRPM8, or TRPA1.

17. The method of claim 14, wherein the composition comprises compound 22 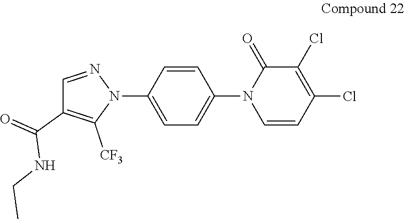 [SMILES: CCNC(=O)c1cnn(-c2ccc(-n3ccc(Cl)c(Cl)c3=O)cc2)c1C(F)(F)F]
or a pharmaceutically acceptable salt thereof.
